(19) United States
(12) Patent Application Publication
(10) Pub. No.: US 2021/0230035 A1
(43) Pub. Date: Jul. 29, 2021

(54) Waste Treatment System

(71) Applicant: Guthalungra Holdings LLC Nevis, West Indies (KN)

(72) Inventor: Ian Geoffrey CUMMINS, Queensland (AU)

(21) Appl. No.: 15/733,966

(22) PCT Filed: May 31, 2019
(86) PCT No.: PCT/AU2019/050560

§ 371 Date: Dec. 1, 2020

(30) Foreign Application Priority Data
Jun. 2, 2018 (AU) 2018901984
Apr. 5, 2019 (AU) 2019901167

(51) Int. Cl. C02F 9/00; C01B 13/10; B01J 19/12; B01J 19/24

(52) U.S. Cl. CPC C02F 9/00; C01B 13/10; B01J 19/123; B01J 19/245; C02F 1/325; C02F 2201/782; C02F 2201/784; C02F 2201/3228; C02F 2301/063; C02F 2201/3227

(57) ABSTRACT

A waste treatment system including at least one treatment chamber through which a fluid to be treated is moved in a fluid flow, the or each treatment chamber including at least one of a number of ultraviolet light sources, and at least one dispersion assembly located at a base of the treatment chamber to disperse at least one sterilizing agent supplied to the treatment chamber in a counter flow direction to the fluid flow.

### TECHNICAL FIELD

The present invention relates generally to waste treatment systems and more particularly to treatment systems utilising ultraviolet light and/or ozone.

### BACKGROUND ART

The treating of fluids such as water for the purpose of purifying or removing contaminants from the fluid has become an increasing problem for growing communities where increasing volumes of effluent or contaminated water is generated. Contaminated water can be generated in domestic, commercial and agricultural situations. Waterways and river systems can also be heavily polluted. Often such water receives primary treatment and is then simply left in settling ponds where solids settle out. However, such settling ponds can require considerable space.

Fluid treatment systems can include systems include use of membranes, such as micro and nanofiltration membranes, or reverse osmosis membranes. However, particularly when heavily polluted fluids are to be purified, such membranes may foul relatively quickly increasing operational costs.

Other fluid treatment systems may involve use of ultraviolet light. Ultraviolet light is a disinfection method for destroying disease-causing organisms in wastewater effluent in onsite wastewater treatment systems. The UV light destroys the genetic material of microorganisms which prevents them from reproducing. However, for the UV light to be effective, the UV radiation must come in direct contact with the microorganisms in the wastewater stream. Constituents allow a hiding place for the pathogenic organisms and shield them from the UV light. If the UV light does not come in direct contact with the constituents of concern, then it is useless. Turbidity, suspended solids, and flow rate of the wastewater must be kept at low levels to ensure proper treatment.

Other fluid treatment systems may involve use of chemicals or flocculants. One type of chemical is ozone, which is an excellent disinfectant with a superior ability to kill viruses and biological contaminants found in water. It is also a very powerful oxidant that can oxidize metals in water such as manganese, iron, and sulphur into insoluble particles, aiding in their filtration and removal from water.

It will be clearly understood that, if a prior art publication is referred to herein, this reference does not constitute an admission that the publication forms part of the common general knowledge in the art in Australia or in any other country.

### SUMMARY OF INVENTION

The present invention is directed to a waste treatment system, which may at least partially overcome at least one of the abovementioned disadvantages or provide the consumer with a useful or commercial choice.

With the foregoing in view, the present invention in one form, resides broadly in a waste treatment system including at least one treatment chamber.

In a first aspect, the present invention relates to a waste treatment system including
at least one treatment chamber through which a fluid to be treated is moved in a fluid flow, the at least one treatment chamber including:
   i. a plurality of ultraviolet light sources configured to irradiate fluid in the at least one treatment chamber with light at a wavelength of from 240 nm to 400 nm; and
   ii. at least one dispersion assembly located at a base of the treatment chamber to disperse ozone supplied to the treatment chamber in a counter flow direction to the fluid flow; and
at least one ozone generation chamber configured to supply ozone to the at least one dispersion assembly, wherein the at least one ozone generation chamber includes a gas inlet for introduction of an oxygen-containing gas, and at least one ultraviolet light source, wherein the at least one ultraviolet light source is configured to irradiate oxygen-containing gas in the at least one ozone generation chamber with light at a wavelength of from 50 nm to 240 nm to thereby produce ozone.

The inventor has surprisingly found that the above arrangement provides a superior fluid treatment effect. Oxidization by ozone in water purification aids in removing taste and odour problems from water much more efficiently than other substances such as chlorine, and ozone itself doesn't produce any odour or taste. In the first aspect of the invention ozone is generated by ultraviolet light treatment of oxygen, which means that there are no byproducts produced during ozone generation. Due to the fact that ozone consists of oxygen, it reverts back to pure oxygen and disappears without a trace after it has been used. Not only does ozone remove microorganisms from the water, but it also halts the accumulation of deposits in pipes and the water system which greatly improves the quality of water.

However, in the first aspect of the invention the at least one treatment chamber includes a plurality of ultraviolet light sources configured to irradiate fluid in the at least one treatment chamber with light at a wavelength of from 240 nm to 400 nm. Such wavelengths are typically used to destroy chemicals such as ozone. However, the inventor has advantageously found that when ozone is dispersed into the treatment chamber, the UV light surprisingly synergistically acts with the ozone to form an extremely powerful decontaminant. Using this system the inventor has been able to very significantly reduce the biological oxygen demand (BOD), chemical oxygen demand (COD), oil and grease content, coliform content, salt content, ammonia content, bromide/bromate content, and also significantly reduce the concentration of chemical contaminants such as dieldrin. The inventor has performed detailed analyses of fluids treated by the system, and the contaminants do not seem to form precipitates or other similar waste products.

Without wishing to be limited by theory, it is believed that the generation of ozone immediately adjacent (often within 1 metre) of the treatment chamber allows a high concentration of ozone to be achieved in the fluid to be treated due to the proximity of formation and the extremely short time before the ozone is used in the treatment.

In a second aspect, the present invention relates to a waste water treatment system including at least one treatment chamber through which a fluid to be treated is moved in a fluid flow. The at least one treatment chamber may include at least one of:
i. a number of ultraviolet light sources; and
ii. at least one dispersion assembly located at a base of the treatment chamber to disperse at least one sterilizing agent supplied to the treatment chamber in a counter flow direction to the fluid flow.

Features of the first and second aspects of the present invention may be as described below.

In one embodiment (and as described in the first aspect), the system includes at least one treatment chamber through which a fluid to be treated is moved in a fluid flow. The at least one treatment chamber includes at least one (preferably a plurality) of ultraviolet light sources. The ultraviolet light sources may be configured (or be operable) to irradiate fluid in the treatment chamber. The light may operate at a wavelength or wavelength band to have a sterilising or disinfecting effect on the fluid to be treated at least reducing harmful bacteria, viruses and other microbes from the fluid. In one embodiment, the light is at a wavelength of from 240 nm to 400 nm. The at least one treatment chamber may include at least one dispersion assembly located at a base of the treatment chamber to disperse a sterilizing agent (such as ozone) supplied to the treatment chamber in a counter flow direction to the fluid flow. The at least one dispersion assembly may disperse the sterilizing agent (such as ozone) in bubbles, especially having a diameter of less than 1000 μm. In the context of the present invention, the treatment chamber in which treatment with both ultraviolet light and sterilizing agent (such as ozone) occurs, for example as described in this paragraph, may be described by the phrase “extreme advanced oxidation” or “hyperoxidation”. In one embodiment, the fluid in said at least one treatment chamber is polarized.

The system may include any suitable number of treatment chambers. In one embodiment, the system includes at least two, three, four, five, six, seven, eight, nine or ten treatment chambers. Fluid may flow through the treatment chambers in series or parallel. In one embodiment, fluid to be treated flows sequentially through the said treatment chambers. In one embodiment, the at least one treatment chamber is a plurality of treatment chambers, wherein fluid to be treated flows through at least two of said plurality of treatment chambers. In another embodiment, the at least one treatment chamber is at least five treatment chambers, wherein fluid to be treated flows sequentially through the at least five treatment chambers.

Having gas from the at least one dispersion assembly flowing in a counter flow direction to the fluid flow advantageously can assist in mixing of the contents of the treatment chamber.

In various embodiments, the present invention is directed to treatment of fluids and gases, heavy metals, minerals and chemical destruction as well as sodium reduction and a method of removing heavy metals and chemicals, which is suited for treating liquids such as water for the purpose of purifying, cleaning or otherwise removing impurities or contaminants in the liquid. In particular, in some embodiments the invention is designed to reduce salt or sodium chloride, some heavy metals, and break down many chemicals from contained in discharge water from many industries and also breakdown many pharmaceuticals which end up in rivers and underground water. This includes the sterilising or disinfecting of biological material. In the context of the present description, sterilization is distinct from disinfection, sanitization, and pasteurization, in that sterilization kills, deactivates, or eliminates all forms of life and other biological agents which are present.

In various aspects, the system of the present invention is applicable in all environments and temperatures, provides chemical free, healthier, cost efficient and more effective solutions to water and gas purification and treatment application worldwide, is modular in construction and where the source water requires further treatment additional reactor compartments can be added to the system as needed. The fluid to be treated may be an aqueous solution, especially a wastewater.

The term “sterilizing agent” as used throughout the specification and claims typically comprises a gas such as ozone, or ozone enriched air. The sterilizing agent may be introduced into the or each treatment chamber in the form of bubbles.

In the present invention, the ultraviolet (UV) light sources will preferably operate at a wavelength or wavelength band to have a sterilising or disinfecting effect on the fluid at least reducing and preferably eliminating harmful bacteria, viruses and other microbes from the fluid. Additionally, at least some of the ultraviolet light sources may be used to create ozone or ozone enriched air which will be supplied to or formed in a treatment chamber and will have a complementary oxidative effect in the fluid to be treated. In one embodiment, at least some of the ultraviolet light sources operate at a wavelength or wavelength band to have a sterilising or disinfecting effect on the fluid to be treated at least reducing harmful bacteria, viruses and other microbes from the fluid. In one embodiment, at least some of the ultraviolet light sources operate at a wavelength or wavelength band to create ozone in the treatment chamber.

In a one embodiment, one or more UV light source may be provided to have a dual effect, namely produce ozone and to have a sterilising or disinfecting effect. One or more UV light source may be provided that produce UV light with two main peaks in the UV light band, one at or around 250 nm or 254 nm for a sterilising or disinfecting effect, and another peak at or around 185 nm for ozone production. However, in a preferred embodiment ultraviolet light within the treatment chamber is only at a wavelength (or wavelength band) of 240 nm to 400 nm (or between 240 nm to 400 nm).

Ozone generation from oxygen exposed to UV light typically occurs at between 160-240 nm. Above 240 nm light will preferably have sterilising or disinfecting effect.

A shortwave, low pressure UV lamp can be used for this purpose. These lamps will typically produce UV light with two main peaks in the UV light band at or around the desired wavelengths.

Importantly, light at wavelengths at or around 254 nm (or generally above approximately 240 nm) will normally adversely affect the levels of ozone in the fluid, normally degrading the ozone by photolysis. Therefore, the light from the ozone generating UV light sources may be located at a different treatment location within the same treatment chamber to the sterilising or disinfecting light sources to maximise the respective effects.

Alternatively, a number of separate treatment chambers may be used, one treatment chamber with the ozone generating UV light sources optimised to emit light waves predominantly at or around 185 nm for ozone production, and an adjacent treatment chamber having sterilising or disinfecting light sources optimised to emit light waves at or around 254 nm for a sterilising or disinfecting effect. In one embodiment, a number of separate treatment chambers are used, at least one treatment chamber with a number of UV light sources which create ozone in the treatment chamber optimised to emit light waves predominantly at or around 185 nm for ozone production, and at least one treatment chamber having a number of the UV light sources which are at a wavelength or wavelength band to have a sterilising or disinfecting effect optimised to emit light waves at or around 254 nm.

Typically, the ozone generating UV light sources are provided prior to the sterilising or disinfecting light sources in the direction of flow of the fluid through the one or more treatment chambers. This will allow the ozone time to react with the fluid and any constituents before the photolytic destruction of the ozone by the light waves at or around 254 nm for a sterilising or disinfecting effect.

A UV light ozone production system is preferred in the present invention to minimise the production of NO₂ when compared to a corona discharge system in which the N₂ is converted to nitric acid.

In a preferred form, a pre-filtration step may be used. Any type of filter can be used, but a simple sand filter may be optimum in terms of simplicity and low cost. In one embodiment, the system does not include a filter.

Preferably, ozone may be produced and supplied to the at least one dispersion assembly (primary ozone creation). This is preferably achieved using atmospheric air as a feed material, generating ozone from the oxygen exposed to UV light at between 160-240 nm. This process may occur in an ozone generation chamber or an ozone generator. The feed pressure for the atmospheric air is preferably low, around 3 psi, as this will allow time for conversion of the oxygen in the feed to be converted to ozone, maximizing the levels of ozone created.

Secondary ozone generation will preferably occur in the treatment chamber using UV light sources optimized to emit light waves predominantly at or around 185 nm for ozone production.

The present invention includes at least one treatment chamber through which a fluid to be treated is pumped. As mentioned above, there will typically be one or more treatment chambers and preferably, a plurality of treatment chambers. Typically, the plurality of treatment chambers is provided in an in-line configuration with the fluid exiting a first treatment chamber entering a second treatment chamber and then exiting the second treatment chamber and entering a third treatment chamber and so on.

The number of treatment chambers provided will typically be determined according to the efficiency of the treatment chambers and/or any required output conditions and/or based on input conditions of the fluid to be treated.

Typically, a number of treatment chambers will be connected in series although the physical location of the treatment chambers need not necessarily be linear. Where a number of treatment chambers are provided, the treatment chambers are connected to allow flow through the treatment chambers in the treatment system and the physical configuration of the treatment chambers is less important.

Where a plurality of treatment chambers is provided, one or more of the treatment chambers are typically activated in order to treat the fluid to be treated. The number and configuration of treatment chambers which are activated can differ. For example, some treatment chambers may be activated whilst others are deactivated, some or all of the treatment chambers may be partially activated that is some of the UV light sources in some or all of the treatment chambers may be activated and some not activated in order to optimise the treatment of the fluid to be treated. The status of each of the UV light sources in all of the treatment chambers are preferably individually adjustable (on/off and/or the capacity of operation from 0 to 100%) in order to obtain the desired treatment effect.

The at least one or each treatment chamber may have an internal surface which is at least partially reflective or reflective to ultraviolet light, especially produced by the ultraviolet light sources. This may be to maximise the use of the UV light waves which are produced.

Each treatment chamber will be shaped and preferably cylindrically shaped. However, in some embodiments, the treatment chamber may be rectangular or even multisided such as square, pentagonal, hexagonal or octagonal shaped. The at least one treatment chamber may include an inlet at a base (or at a lower portion or position) of the treatment chamber. The at least one treatment chamber may include an outlet at a top (or at an upper portion or position) of the treatment chamber. Where two treatment chambers are in fluid connection, a connector may connect the outlet of a first treatment chamber with the inlet of a second treatment chamber.

In the embodiments where more than one treatment chamber is provided, appropriate connection piping to connect the treatment chambers is also preferably provided. The connection piping may take any physical configuration. For example, in some embodiments, connection piping may be provided to connect an outlet at the base or lower portion of a first treatment chamber with an inlet at an upper position of a second, adjacent treatment chamber. This connection piping may be angled or alternatively, may be formed from a number of portions, for example a first portion which is substantially horizontal with a second portion which is substantially vertical or vice versa. In one embodiment, the system includes at least one connector conduit to an outlet at a base of a first treatment chamber with an inlet at an upper position of a second, adjacent treatment chamber.

The flow direction of the fluid to treated through each treatment chamber is in a direction which is opposite to the direction in which the at least one sterilising agent is dispersed into or through the treatment chamber. Generally, the dispersion assembly is located at the base of a treatment chamber and therefore, the fluid will typically be provided into the treatment chamber through an inlet located at an upper portion of the treatment chamber and exit the treatment chamber through an outlet located at or towards the bottom of the treatment chamber.

One or more waste outlets will typically be provided in each treatment chamber. The specific location of a waste outlet in each treatment chamber would generally be dependent upon the type of waste which is to be removed. For example, any waste which may be contained in a froth or similar will typically be removed from an upper portion of the treatment chamber and therefore, the waste outlet will normally be in an upper portion of the treatment chamber. Similarly, any waste in the form of sediment will typically be removed from a lower portion of the treatment container.

Preferably, any treatment container which is designed to have or promote sedimentation will typically not include a dispersion assembly located at the base of the treatment chamber as this would tend to keep the sediment in suspension rather than allowing sedimentation to occur.

In a particularly preferred embodiment of the present invention the treatment chambers will be provided in one or more sets of two treatment chambers with an ozone generating chamber followed by a UV sterilizing chamber. More than one set of two treatment chambers can be provided and typically, the treatment chambers will alternate in treatment configuration that is an ozone generating chamber followed by a UV sterilizing chamber followed by an ozone generating chamber followed by a UV sterilizing chamber and so on.

A drain valve may be provided in a lower portion of each of the treatment chambers in order to drain fluid from the treatment chamber.

As mentioned above, each treatment chamber will typically be in fluid connection with an adjacent treatment chamber where more than one treatment chamber is provided in series. A single connection may be provided or more than one fluid connection can be provided between linked treatment chambers.

The fluid connection will normally be provided by one or more connector conduits extending between adjacent treatment chambers. Additional treatment components may be provided in association with the connector conduit.

For example, one or more UV light sources may be provided within a connector conduit such that fluid flowing through the connector conduit will flow past of the UV light sources thereby being treated by the one or more UV light sources.

One or more chlorinators or similar components may be provided in association with and/or within a connector conduit such that fluid flowing through the connector conduit can be treated with chlorine produced by the one or more chlorinators.

Each treatment chamber will typically be provided with a top wall and a bottom wall and at least one side wall extending between the top wall in the bottom wall. Preferably, the top wall in bottom wall are provided as or with a removable top and/or bottom to provide or allow access to an internal part of each of the treatment chambers.

The treatment system of the present invention may be provided within a housing to allow easy transport. In particular, a treatment system according to the present invention may be provided within one or more shipping containers, or in or in association with a floating vessel or boat designed to travel across a body of fluid to be treated. If ancillary equipment such as filters or pumps and the like are provided, these can be provided in the same housing as the treatment system or alternatively, one or more additional housings may be provided to house ancillary equipment which can then be associated with the housing which houses the treatment system.

The treatment system of the present invention may include a number of ultraviolet light sources provided within at least one treatment chamber. The ultraviolet light sources can be provided in the same treatment chamber as the at least one dispersion apparatus or ultraviolet light source, or may be provided in one or more treatment chambers which are separate from one or more treatment chambers housing the at least one dispersion apparatus.

Preferably, a number of ultraviolet light sources are provided in a number of different treatment chambers. As mentioned above, a treatment chamber may be provided with a number of ultraviolet light sources with or without a dispersion assembly. Preferably, the light sources will be configured to provide different wavelengths of light to the fluid to be treated. The light sources may treat the fluid directly and/or create secondary materials such as ozone for example that in turn treat the fluid, typically by acting on the materials carried by the fluid which the treatment is designed to remove.

In one embodiment, more than one treatment chamber is provided, at least one treatment chamber having a number of ultraviolet light sources and at least one treatment chamber having at least one dispersion assembly. In another embodiment, at least one of the treatment chambers includes a number of ultraviolet light sources and at least one dispersion assembly.

In one embodiment, the treatment chamber includes a plurality of ultraviolet light sources. The plurality of ultraviolet light sources may be configured to (or be operable to) irradiate fluid in the treatment chamber. The treatment chamber may include any suitable number of ultraviolet light sources. In one embodiment, the at least one treatment chamber includes at least one, two, three, four, five, six, seven, eight, nine or ten ultraviolet light sources. The at least one treatment chamber may include less than 30 ultraviolet light sources. The total wattage of the ultraviolet light sources in a single treatment chamber may be from 100 to 4,500 watts, especially from 300 to 4,000 watts, more especially from 500 to 3,500 watts, or from 1,000 to 3,500 watts.

According to one embodiment, the ultraviolet light sources will provide light waves at at least approximately 180 nm and approximately 254 nm. Without wishing to be limited by theory, light sources at approximately 180 nm will preferably create ozone and light sources at approximately 254 nm will preferably treat biologics for example.

In one embodiment, the UV light sources provide light at about 50 nm to about 240 nm, or about 50 nm to about 210 nm, or about 130 nm to about 210 nm, especially from about 150 nm to about 200 nm, more especially from about 170 nm to about 200 nm or about 170 nm to about 190 nm, most especially form about 180 nm to about 190 nm. Such light sources may especially be present in the at least one ozone generation chamber.

In another embodiment, the UV light sources provide light at about 210 nm to 400 nm, or about 210 nm to about 310 nm, especially about 220 nm to about 290 nm, more especially about 240 nm to about 270 nm, most especially about 240 nm to about 260 nm or about 250 to about 260 nm. In another embodiment, the UV light sources provide light at about 240 nm to 400 nm, or about 240 nm to about 310 nm, especially about 240 nm to about 290 nm, more especially about 240 nm to about 270 nm, most especially about 240 nm to about 260 nm or about 250 to about 260 nm. Such light sources may especially be present in the at least one treatment chamber.

Light may be provided at more than these particular wavelengths. For example, UV light may be provided at UV-A wavelengths (315-400 nm), UV-B wavelengths (280-315 nm) and/or UV-C wavelengths (100-280 nm).

Light waves which are introduced into the fluid at one wavelength may also be split into light waves of lesser wavelength for example through interactions with materials carried by the fluid to be treated and/or bubbles for example which introduced into the fluid to be treated, for example by the at least one dispersion apparatus.

The UV light sources may be provided in any configuration. For example, as mentioned above, a number of ultraviolet light sources may be provided in a first treatment chamber and then an adjacent treatment chamber may contain a dispersion assembly without UV light sources and/or light sources may be provided in all treatment chambers in a treatment system.

The UV light sources may be grouped within a treatment chamber. For example, the light sources may be physically grouped within a treatment chamber in a bunched configuration in order to enhance the treatment of the fluid. The light sources may be grouped within a treatment chamber or in different treatment chambers according to a particular wavelength which is desired in an area of a treatment chamber or in a treatment chamber. For example, a number of light sources emitting light waves at or around 180 nm may be grouped in a particular location in a treatment chamber with a number of light sources emitting light waves at or around 254 nm grouped in a particular location that is different or separated from the grouping at or around 180 nm.

As mentioned above, any material or bodies within the fluid to be treated may further disperse or divide the UV light such as for example the UV light wavelengths may be split by bubbles from the dispersion apparatus and/or the bubbles may spread the light through the fluid to be treated.

The UV light sources may have any orientation relative to the treatment chamber but will typically be either a vertically oriented or horizontally oriented. UV light sources of a combination of orientations may be provided in a single treatment chamber or each treatment chamber may be limited to UV light sources of a particular orientation. The UV light sources may extend between the side wall(s) of the chamber.

The UV light sources may be spaced approximately equal across the volume of the treatment chamber in order to ensure treatment and/or grouped as outlined above.

A said chamber may include any number of UV light sources. At least one (or each) chamber may include from 5 to 100 UV light sources, especially from 5 to 50 UV light sources, more especially from 10 to 40 UV light sources, most especially from 15 to 40 UV light sources. Each said UV light source may be from 60 W to 150 W, especially from about 70 W to about 120 W, more especially from about 80 W to 100 W. The UV light sources may be dispersed throughout the or each chamber. The UV light sources may be layered within the or each chamber. Each said layer of UV light sources may include UV light sources extending in a plurality of different directions. The or each chamber may include any suitable number of layers of UV light sources, especially 2, 3, 4 or 5 layers. Each said layer may include 3, 4, 5, 6, 7, 8, 9 or 10 UV light sources. The system may include a voltage regulator in electrical connection with the UV light sources so as to provide consistent voltage.

The housing may be provided which is divided using one or more divider panels in order to produce an optimal flow of fluid through the housing. For example, by providing a rectangular housing with a series of divider panels with each alternate divider panel spaced from the floor and each other alternate divider panel attached to the floor but spaced from the top of the body of fluid, the divider panels can cause an under or over flow pattern through the housing.

Where divider panels are provided, the divider panels may be removable and/or movable within the housing. It is preferred that any divider panels are planar but any shape could be used.

Typically, pipework will be provided relative to a housing that is provided with divider panels in order to assist with the flow of either the fluid to be treated or other fluids which are provided to the treatment chamber such as for example a least one sterilising agent.

At least one treatment chamber in the treatment system of the present invention will typically include at least one dispersion assembly located at a base of one or more treatment chambers in order to disperse at least one sterilising agent supplied to the treatment chamber through the fluid to be treated. In a particularly preferred form, the sterilising agent will be ozone. In this way, ozone or gas charged with ozone can be provided to a treatment chamber in order to treat fluid, typically by oxidation of components or material being carried by the fluid to be treated.

At least one dispersion assembly may be provided in each treatment chamber within the system or only in one or more treatment chambers. In a particularly preferred embodiment, a number of treatment chambers are provided with each alternating treatment chamber provided with a number of ultraviolet light sources and each other alternating treatment chamber provided with at least one dispersion assembly. However in some embodiments, a dispersion assembly can be provided in every treatment chamber with the UV light sources provided in only some, preferably alternating treatment chambers within the system.

In some configurations, a treatment chamber will have ozone created in situ within the treatment chamber through the provision of UV light sources within the treatment chamber and at least one dispersion assembly will add ozone or air enriched with ozone to the same treatment chamber in order to enhance treatment. However, in a preferred embodiment the at least one treatment chamber is not configured to produce ozone, instead ozone is supplied to the at least one treatment chamber from an ozone generation chamber.

Additional treatment chambers may be provided without any ultraviolet light sources or dispersion assemblies such as for example to allow sedimentation of material which has been treated by the UV light sources and/or dispersion assembly to settle to allow removal from the fluid to be treated.

The means for introducing the sterilizing agent into the primary treatment chamber may comprise one or more gas outlets, the one or more gas outlets comprising one or more of air stones, a gas permeable pipe or pipes, a diffuser or diffusers or an external venturi or venturis communicating with the primary treatment chamber and a source of the sterilizing agent. In one embodiment, the at least one dispersion assembly comprises one or more gas outlets, one or more air stones, a gas permeable pipe or pipes, a diffuser or diffusers or an external venturi or venturis communicating with the treatment chamber and a source of the sterilizing agent.

Preferably, the sterilizing agent is introduced in the form of bubbles, especially from the dispersion assembly. The bubbles will typically be introduced in the form of microbubbles or nanobubbles, for example the dispersion assembly may include a microbubble and/or a nanobubbler. Without wishing to be limited by theory, the bubbles will preferably act to increase the surface area of the sterilizing agent. In one embodiment, the bubbles have a diameter of less than 1000 μm. In other embodiments, the bubbles have a diameter of less than 500, 400, 300, 200, 100 or 50 μm. As used herein, these diameters refer to the diameter of the bubbles at the point of release from the dispersion assembly; the bubbles may coalesce into larger bubbles as they move through the treatment chamber.

The means at the upper end of the chamber for removing waste may comprise an inverted U-shaped trap and/or a venturi unit.

Preferably, the system may further compromise a means at the upper end of any one or more of the treatment chambers for removing waste in the liquid conveyed by the bubbles upwardly through the chamber. The means at the upper end of the chamber for removing waste may compromise an outlet, preferably located above the preferred upper inlet for fluid to be treated preferably to take advantage of any flotation of waste materials, especially particulate or agglomerated material that may be carried upward by the bubbles and in particular, any waste materials in froth that may be formed. Each treatment chamber will preferably have a waste outlet and preferably, the respective waste outlets are associated with a common waste carriage conduit. In a preferred form, the common waste carriage conduit may have air or a gas pumped therethrough to create a suction effect at the waste outlet to assist with extraction of the waste materials.

In one embodiment, at least one or each chamber may include at least one suction source. The at least one suction source may be located at one end, especially an upper end of the at least one chamber. The suction source may reduce the pressure in the chamber (and thereby remove excess gases). The suction source may be a venturi. In one embodiment, each chamber includes a suction source. The function of the suction source is preferably to remove any gaseous material that is created or which exits a liquid body in the chamber, particularly if injected at a lower part of the chamber. The suction source will typically be located above any liquid inlet.

A tube or similar may be used in association with any one or more of the plurality of UV light sources. The UV light sources are preferably elongate and/or are provided in an elongate tube to allow the light to pass and/or to disperse the light. Preferably, the tube will be or include a quartz tube. The preferred quartz tube can be inserted into the treatment chamber horizontally or vertically, may extend across the width of the chamber or vertically substantially over the height of the treatment chamber. Preferably, a quartz tube or similar may extend substantially the length of the UV light source and the tube preferably protects the ultraviolet light from the fluid in the treatment chamber. The quartz tube may also surround the UV light sources and allow space for ozone enriched air to be produced within the quartz tube in certain embodiments. As this ozone enriched air is transparent, the UV light waves may freely pass through the ozone enriched air and into the fluid.

Preferably, a quartz tube or similar may extend substantially the length of the connection conduit and the tube protects the ultraviolet light from the fluid in the connection conduit.

Preferably, the tube may be manufactured from clear quartz or from any like materials; the tube allows the light wave from the ultraviolet light to interact with the ozone bubbles and reflect and react around the chamber.

The UV light sources may also be or include LED lights.

Preferably, the system may further comprise an end cap which is releasable attached to the top of each treatment chamber.

The tubes are preferably mounted through a sidewall and/or end cap and/or bottom mall depending upon their orientation.

Preferably, the waste removing means of each treatment chamber is connected to one or more waste pipes that is common. The lower ends of the treatment chambers may be selectively connectable to one or more common drainage pipes or ducts via control valves to allow drainage of the chambers.

Preferably, the system may further comprise at least one pre-filter located in the inlet line before the first chamber for removing any large materials from the fluid to be treated.

Preferably, the system may further comprise a U-shaped bend or trap to retain a level of water of fluid in the or each waste outlet to prevent any gas, particularly ozone gas or air charged with ozone gas, leaking into the waste.

Preferably, a combination of different wavelengths lamps may be used in the ozone production to produce ozone gas.

Preferably, the at least one dispersion assembly is associated with an ozone generator using ultraviolet light sources operating at a wavelength or wavelength band to create ozone or ozone enriched air which is then supplied to the at least one dispersion assembly.

Preferably, the UV light sources which create ozone in the treatment chamber are located first in the fluid flow followed by and separated from the UV light sources which are at a wavelength or wavelength band to have a sterilising or disinfecting effect.

Preferably, the ozone generator uses atmospheric air as a feed material at a feed pressure for the atmospheric air of approximately 3 psi to 5 psi, generating ozone from oxygen in the atmospheric air exposed to UV light at between 160-240 nm.

Preferably, the or each treatment chamber has an internal surface which is at least partially reflective in order to maximise the use of UV light waves which are produced.

Preferably, at least one connector conduit to an outlet at a base of a first treatment chamber with an inlet at an upper position of a second, adjacent treatment chamber.

Preferably, one or more chlorinators are provided within the connector conduit such that fluid flowing through the connector conduit is treated with chlorine produced by the one or more chlorinators.

Preferably, the treatment chamber is defined in a housing which is divided using one or more movable divider panels in order to produce an optimal flow of fluid through the housing.

Preferably, a number of treatment chambers are provided with each alternating treatment chamber provided with a number of ultraviolet light sources and each other alternating treatment chamber provided with at least one dispersion assembly.

Preferably, a treatment chamber has ozone created in situ within the treatment chamber through the provision of UV light sources within the treatment chamber and at least one dispersion assembly to add ozone or air enriched with ozone to the treatment chamber in order to enhance treatment.

Each chamber may be of any suitable volume. In one embodiment, each chamber has a volume of from 0.2 m³ to about 5 m³, especially from about 0.5 m³ to about 2 m³, more especially from about 1 m³ to about 1.5 m³. In the examples illustrated below each chamber typically has a volume of about 1 m³ to about 1.5 m³.

The system may be operated at any suitable flow rate, and the selection of flow rate will depend upon the cleanliness of the water. Cleaner liquids may be treated at a faster rate. In a system including 10 of the at least one treatment chamber (or extreme advanced oxidation) and 10 ozone treatment chambers (as discussed below), a typical flow rate would be from 0.7 m³ per minute to about 3.5 m³ per minute (about 1,000 to about 5,000 m³/day).

The system preferably operates at fluid to be treated feed pressure below 5 psi. The treatment system of the invention may employ one or multiple treatment chambers.

Treatment chambers can be used to treat fluid to be treated in particular, generally liquid or wastewater will be treated in order to provide clean water as a result.

The additional ozone or air enriched with ozone which may be used according to the present invention is typically created on-site, generally close to the injection location of the ozone or air enriched with ozone. According to a preferred embodiment, the ozone or air enriched with ozone is generally formed using UV light sources as well.

In order to be clear, in a preferred embodiment, typically air enriched with ozone is preferably created by passing atmospheric air past a UV light source configured to emit light waves at one or more wavelengths conducive to the creation of ozone and then this ozone is preferably passed to at least one dispersion assembly located in a treatment chamber to treat liquid.

The present invention can be configured to treat air or other gases with appropriate changes to the configuration within the scope of the invention.

In one embodiment, the system may also include at least one ozone treatment chamber (or sterilizing agent treatment chamber) through which a fluid to be treated is moved in a fluid flow. The at least one ozone treatment chamber (or sterilizing agent treatment chamber) may include at least one dispersion assembly located at a base of the ozone treatment chamber (or sterilizing agent treatment chamber) to disperse ozone (or sterilizing agent) supplied to the ozone treatment chamber in a counter flow direction to the fluid flow. The at least one dispersion assembly may disperse the sterilizing agent (such as ozone) in bubbles, especially having a diameter of less than 1000 Unlike the at least one treatment chamber described above, the at least one ozone treatment chamber (or sterilizing agent treatment chamber) typically does not include any ultraviolet light sources. The at least one ozone generation chamber may be configured to supply ozone to the at least one dispersion assembly in the ozone treatment chamber. Fluid to be treated may move through the at least one treatment chamber and the at least one ozone treatment chamber. Context permitting, features of the ozone treatment chamber (or sterilizing agent treatment chamber) may be as described above for the at least one treatment chamber.

In one embodiment, the system may also include at least one ultraviolet light treatment chamber through which a fluid to be treated is moved in a fluid flow. The at least one ultraviolet light treatment chamber may include at least one (preferably a plurality) of ultraviolet light sources. The ultraviolet light sources may be configured (or be operable) to irradiate fluid in the ultraviolet light treatment chamber. The light may operate at a wavelength or wavelength band to have a sterilising or disinfecting effect on the fluid to be treated at least reducing harmful bacteria, viruses and other microbes from the fluid. In one embodiment, the light is at a wavelength of from 240 nm to 400 nm. Fluid to be treated may move through the at least one treatment chamber and the at least one ultraviolet light treatment chamber. Unlike the at least one treatment chamber described above, the at least one ultraviolet light treatment chamber typically does not include a dispersion assembly. The ultraviolet light irradiated in the ultraviolet light treatment chamber may interact with residual ozone or sterilizing agent in the fluid flow. In the context of the present invention, the ultraviolet light treatment chamber in which treatment with both ultraviolet light occurs without use of a dispersion assembly, for example as described in this paragraph, may be described by the phrase “advanced oxidation”. Context permitting, features of the ultraviolet light treatment may be as described above for the at least one treatment chamber.

The system of the present invention may include at least one treatment chamber, at least one ozone treatment chamber (or sterilizing agent treatment chamber) and/or at least one ultraviolet light treatment chamber in any suitable combination. The system may include any number of at least one treatment chambers. For example, the system may include at least two, three, four, five, six, seven, eight, nine or ten treatment chambers. The said treatment chambers may be connected in series or parallel, especially in series.

Similarly, the system may include any suitable number of at least one ozone (or sterilizing agent) treatment chambers. For example, the system may include at least two, three, four, five, six, seven, eight, nine or ten ozone (or sterilizing agent) treatment chambers. The said ozone (or sterilizing agent) treatment chambers may be connected in series or parallel, especially in series. In one embodiment, fluid to be treated moves through the at least one ozone treatment chamber before moving through the at least one treatment chamber. Advantageously, use of at least one ozone (or sterilizing agent) treatment chamber immediately before the at least one treatment chamber in the fluid flow may elevate the concentration of ozone (or sterilizing agent) in the fluid flow within the at least one treatment chamber, which thereby may enhance the reaction in the at least one treatment chamber.

The system may also include any suitable number of at least one ultraviolet light treatment chambers. For example, the system may include at least two, three, four, five, six, seven, eight, nine or ten ultraviolet light treatment chambers. The said ultraviolet light treatment chambers may be connected in series or parallel, especially in series. In one embodiment, fluid to be treated moves through the at least one treatment chamber before moving through the at least one ultraviolet light treatment chamber. Advantageously, use of at least one ultraviolet light treatment chamber after the at least one treatment chamber or the at least one ozone (or sterilizing agent) treatment chamber in the fluid flow may decrease the concentration of ozone (or sterilizing agent) in the fluid flow, for example, before the fluid exits the system.

The system may also include a plurality of groups of the at least one treatment chamber, the at least one ozone treatment chamber (or sterilizing agent treatment chamber) and/or the at least one ultraviolet light treatment chamber. For example, a said (or each) group may include at least one treatment chamber, at least one ozone treatment chamber (or sterilizing agent treatment chamber) and at least one ultraviolet light treatment chamber. Alternatively, a said (or each) group may include at least one treatment chamber, and at least one ozone treatment chamber (or sterilizing agent treatment chamber). Alternatively, a said (or each) group may include at least one treatment chamber, and at least one ultraviolet light treatment chamber. For example, a said (or each) group may include one, two three, four or five treatment chambers. A said (or each) group may include one, two three, four or five ozone treatment chambers (or sterilizing agent treatment chamber). A said (or each) group may include one, two three, four or five ultraviolet light treatment chambers. Where a group includes more than one treatment chamber, ozone treatment chamber (or sterilizing agent treatment chamber) or ultraviolet light treatment chamber, multiples of the same type of chamber may be connected in series or parallel, especially in parallel. The system may include at least two groups, especially two, three, four, five, six, seven, eight, nine or ten groups.

The system may also include at least one ozone generation chamber (or at least one ozone generator). In one embodiment, the ozone generator includes at least one ozone generation chamber.

The at least one ozone generation chamber may be configured to supply ozone to the at least one dispersion assembly. In one embodiment, the system includes an ozone generation chamber for each dispersion assembly. However, in other embodiments a single ozone generation chamber may supply ozone to more than one dispersion assembly. The system may include a plurality of ozone generation chambers.

The at least one ozone generation chamber may include a gas inlet for introduction of an oxygen-containing gas. The oxygen containing gas may be air, oxygen, or oxygen enriched air. The at least one ozone generation chamber may include a gas outlet in fluid connection with at least one dispersion assembly. The at least one ozone generation chamber may include at least one ultraviolet light source. The at least one ultraviolet light source may be configured to irradiate oxygen-containing gas in the at least one ozone generation chamber to thereby produce ozone.

In one embodiment, the at least one ozone generation chamber includes a plurality of ultraviolet light sources. The at least one ozone generation chamber may include any suitable number of ultraviolet light sources. In one embodiment, the at least one at least one ozone generation chamber includes at least one, two, three, four, five, six, seven, eight, nine or ten ultraviolet light sources. The at least one at least one ozone generation chamber may include less than 30 ultraviolet light sources. At least one (or each) ozone generation chamber may include from 5 to 100 UV light sources, especially from 5 to 50 UV light sources, more especially from 10 to 40 UV light sources, most especially from 15 to 40 UV light sources.

The total wattage of the ultraviolet light sources in a single at least one ozone generation chamber may be from 100 to 4,500 watts, especially from 300 to 4,000 watts, more especially from 500 to 3,500 watts, or from 1,000 to 3,500 watts. Each said UV light source may be from 60 W to 150 W, especially from about 70 W to about 120 W, more especially from about 80 W to 100 W. The UV light sources may be dispersed throughout the or each chamber. The UV light sources may be layered within the or each chamber. Each said layer of UV light sources may include UV light sources extending in a plurality of different directions. The or each chamber may include any suitable number of layers of UV light sources, especially 2, 3, 4 or 5 layers. Each said layer may include 3, 4, 5, 6, 7, 8, 9 or 10 UV light sources.

The ultraviolet light sources in the at least one ozone generation chamber may be in any suitable orientation or position. Such orientations or positions may be as described above for the at least one treatment chamber.

In one embodiment, the UV light sources provide light at about 50 nm to about 240 nm, or about 50 nm to about 210 nm, or about 130 nm to about 210 nm, especially from about 150 nm to about 200 nm, more especially from about 170 nm to about 200 nm or about 170 nm to about 190 nm, most especially form about 180 nm to about 190 nm. Such light sources may especially be present in the at least one ozone generation chamber.

In one embodiment, the at least one ozone generation chamber is configured to supply gas including at least 10 ppm ozone to the at least one dispersion assembly, especially at least 20 ppm ozone, or at least 30 ppm ozone, or at least 40 ppm ozone, or at least 50 ppm ozone.

In a third aspect, the present invention provides a method of treating a fluid, the method including passing fluid through the waste treatment system of the first or second aspects. Features of the third aspect of the present invention may be as described for the first and second aspects of the present invention.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 shows an isometric view of a system of a preferred embodiment with angled water transfer embodiments.

FIG. 2 shows an isometric view of a system of a preferred embodiment with curved, horizontal and vertical transfer pipes.

FIG. 3 is a top view of the system illustrated in FIG. 2.

FIG. 4 shows a top view of the system illustrated in FIG. 1.

FIG. 5 is a top view of an embodiment similar to FIG. 1 but with alternating vertical-horizontal transfer pipes.

FIG. 6 is a side view of the configuration shown in FIG. 5.

FIG. 7 is shows a rear side view of an alternative configuration.

FIG. 8 is a side view of the configuration illustrated in FIG. 1.

FIG. 9 is a top view of an alternative configuration to FIG. 1 with every second tank fitted with vertical and horizontal UV tubes.

FIG. 10 is a side view of a further alternative configuration.

FIG. 11 is a top view showing vertical and horizontal UV tubes in every second tank.

FIG. 12 is an isometric view of a further alternative configuration.

FIG. 13 is a side view of two tanks according to a preferred configuration.

FIG. 14 is an end view of a single tank of a preferred embodiment.

FIG. 15 is a side view of an alternative tank showing internals.

FIG. 16 is a top view of the embodiment shown in FIG. 14.

FIG. 17 is a top view of the embodiment shown in FIG. 15

FIG. 18 is an isometric view of a preferred two-tank processing embodiment with connections.

FIG. 19 is a top view of a tank showing UV bulbs in each layer in a preferred embodiment.

FIG. 20 is a top view is an embodiment showing the horizontal-vertical advance oxidation transfer pipe on the alternating side of each tank two tank processing embodiment from FIG. 18.

FIG. 21 is an isometric view of the embodiment in FIG. 20.

FIG. 22 is a side view of the horizontal-vertical chambers of a preferred embodiment.

FIG. 23 is a side view showing an alternative embodiment.

FIG. 24 is a side view of the angled transfer system embodiment.

FIG. 25 is a partially transparent isometric view of a preferred system showing internal components of the treatment tanks.

FIG. 26 is a side view of a two-treatment tank embodiment with inlet pipe with an angled transfer pipe fitted with a chlorinator.

FIG. 27 is a front elevation view of a chlorinator with ultraviolet bulbs according to an embodiment.

FIG. 28 is a front elevation view of the chlorinator of FIG. 27 with the bulbs removed.

FIG. 29 is isometric view of a preferred treatment system fitted in a shipping container as a housing for transport according to an embodiment.

FIG. 30 is a side view of a ten-tank, 5-pair embodiment showing UV angled transfer pipes with horizontal UV in every second tank.

FIG. 31 is a schematic view of a 40-foot shipping container with a filter shipping container at either end for pre-filtering and post filtering according to a preferred embodiment.

FIG. 32 shows a system of a preferred embodiment configured for gas destruction or air purification according to a preferred embodiment.

FIG. 33 shows a three-chamber hexagonal configuration of a preferred embodiment.

FIG. 34 is a side view of the configuration illustrated in FIG. 33.

FIG. 35 is a side view of a treatment system of an embodiment.

FIG. 36 is a top view of the configuration illustrated in FIG. 35.

FIG. 37 shows the chemical structure of dioxin (without end Chlorines).

FIG. 38 shows the molecular structure of dioxin post treatment in a system of the present invention.

FIG. 39 is a schematic view of a treatment system including treatment chambers of an embodiment of the present invention for reducing the salinity of seawater for use in a boiler.

FIG. 40 is a schematic view of a treatment system including treatment chambers of an embodiment of the present invention for a coal-fired power station.

FIG. 41 is an isometric view of a piping arrangement for a system of a preferred embodiment.

FIG. 42 is an isometric view of flow control panels in single sections and in multiple sections for any water or fluid height adjustment in a treatment chamber of a preferred embodiment.

FIG. 43 shows the configuration in FIG. 41 with grooves or slide sections built into the main body to allow for the panels shown in FIG. 42 to define one or more treatment chambers.

FIG. 44 is a top view of one part of the configuration illustrated in FIG. 43.

FIG. 45 is a top view of the configuration illustrated in FIG. 43.

FIG. 46 is a partially transparent isometric view of configuration shown in FIG. 43 with piping removed and dividers and UV tubes in place.

FIG. 47 is a top view of the configuration illustrated in FIG. 46.

FIG. 48 is a schematic side view of the configuration shown in FIG. 46 showing the flow path.

FIG. 49 is an isometric view of a rectangular housing from the embodiment illustrated in FIG. 46 with dividers having disruption baffles.

FIG. 50 is an isometric view of a rectangular housing side mounted, planar dividers according o a preferred embodiment.

FIG. 51 is a side view of the configuration illustrated in FIG. 50.

FIG. 52 is a top view of two polarization chambers in series configuration according to a preferred embodiment of the present invention.

FIG. 53 is a side view of the configuration illustrated in FIG. 52 showing internal components.

FIG. 54 is an isometric view of a waterborne vessel configuration according to an embodiment.

FIG. 55 is an isometric view of a waterborne vessel configuration according to a further embodiment.

FIG. 56 is an end view of a vessel of FIGS. 54 and 55 with a central portion with UV treatment lamps in a submerged operating position.

FIG. 57 is an overhead view of an alternative embodiment including four shipping containers with centrally located treatment system according to an embodiment.

FIG. 58 is an end view of a vessel of FIGS. 54 and 55 with a central portion with air stones and delivery pipes.

FIG. 59 is an end view of a vessel of FIGS. 54 and 55 with a central portion in a lifted position with inlet pipes raised above.

FIG. 60 is a side view of a vessel of FIGS. 54 and 55 with a central portion with UV treatment lamps in a submerged operating position

FIG. 61 is an isometric view of an alternative treatment system of an embodiment with ultraviolet bulbs in racks compressed together.

FIG. 62 is an overhead view the configuration shown in FIG. 55 with a central portion with UV treatment lamps.

FIG. 63 is an overhead view of an alternative configuration with laterally extending UV light assemblies.

FIG. 64 is an isometric view of a vessel embodiment with the treatment system of including ultraviolet bulbs in racks compressed together.

FIG. 65 is a top view of an alternative embodiment with three sets of ultraviolet bulbs in racks compressed together.

FIG. 66 is an overhead view of an alternative configuration with laterally extending UV light assemblies with some ultraviolet lights in the off and some ultraviolet lights turned on.

FIG. 67 is a side view of a vessel of an embodiment with the lifting platform in the raised position.

FIG. 68 is a partially transparent isometric view of the configuration shown in FIG. 67.

FIG. 69 is an overhead view of an alternative configuration with laterally extending UV light assemblies in racks in different positions.

FIG. 70 is an isometric view of an alternative embodiment with vertically extending UV light assemblies past which fluid moves.

FIG. 71 is an ultraviolet light assembly to create ozone according to a preferred embodiment.

FIG. 72 is a top view of an embodiment including laterally extending UV light assemblies with short transverse reflective plates.

FIG. 73 is a top view of an embodiment including laterally extending UV light assemblies with elongate transverse reflective plates.

FIG. 74 is a schematic illustration of a horizontal tube bundle to create ozone from compressed air or oxygen according to an embodiment.

FIG. 75 is a detailed view of the configuration illustrated in FIG. 74.

FIG. 76 is a schematic illustration of a vertical tube bundle to create ozone from compressed air or oxygen according to an embodiment.

FIG. 77 is a top view of the ozone formation configuration for FIG. 74 used to feed ozone to the liquid treatment chambers of FIG. 45.

FIG. 78 is a front view showing six (6) 40 ft. shipping containers or similar vessels stacked vertically with six (6) ft. shipping container attached at one end to form a power generation system of an embodiment.

FIG. 79 is a top view of a modernized sewage treatment plant utilizing a treatment system of an embodiment of the invention.

FIG. 80 is an isometric view of a moving belt filter to filter water prior to treatment with an embodiment of the present invention.

FIG. 81 is a side view of the belt filter illustrated in FIG. 80.

FIG. 82 is an isometric view of the belt filter illustrated in FIG. 80 in a housing.

FIG. 83 is a top view of a C-section continuous polarization reactor according to a preferred embodiment of the present invention.

FIG. 84 is an isometric view of the reactor in FIG. 83.

FIG. 85 is a top view of a round continuous polarization reactor according to a preferred embodiment of the present invention.

FIG. 86 is a side view of the embodiment illustrated in FIG. 85.

FIG. 87 is a top view of a 40 ft container embodiment fitted with internal surrounding water tanks with a walkway down the middle and four treatment stations according to an embodiment of the present invention.

FIG. 88 is a top view of the configuration shown in FIG. 87 with a single treatment station according to an embodiment of the present invention.

FIG. 89 is a side view of a 40 ft container converted to a temperature control aquaculture system with a single treatment station according to an embodiment of the present invention.

FIG. 90 is a side view of a 40 ft container converted to a temperature control aquaculture system with a multiple treatment stations according to an embodiment of the present invention.

FIG. 91 shows a block of 100 containerized units according to a preferred embodiment to hold aquaculture systems and containers hold water treatment systems.

FIG. 92 is an overhead view of an alternative aquaculture system using recirculating water according to an embodiment.

FIG. 93 is an isometric view of a treatment system of a preferred embodiment.

FIG. 94 shows the frame construction of the embodiment illustrated in FIG. 93.

FIG. 95 is an exploded view of a treatment chamber for treating water or fluids with ozone production unit according to a preferred embodiment.

FIG. 96 is an isometric view of a treatment chamber suitable for treating water or fluids according to an embodiment.

FIG. 97 is an exploded view of the configuration illustrated in FIG. 96.

FIG. 98 is a side view of the configuration illustrated in FIG. 96

FIG. 99 is a larger scale version of the configuration illustrated in FIG. 96.

FIG. 100 shows the configuration illustrated in FIG. 99 with the shutter removed.

FIG. 101 is a top view of the configuration illustrated in FIG. 100.

FIG. 102 is a side view of a system created from multiple units as illustrated in FIG. 99 linked together.

FIG. 103 shows the laboratory results for treatment of water flowing from an underground gold and copper mine by an embodiment of the present invention.

FIG. 104 shows the laboratory testing of milk waste from an ice cream factory treated by an embodiment of the present invention.

FIG. 105 shows the testing of biodiesel treated by an embodiment of the present invention.

FIG. 106 shows the testing of sewage water treated by an embodiment of the present invention.

FIG. 107 shows a comparison of lake water before and after treatment by an embodiment of the present invention.

FIG. 107A shows the test results of the water in FIG. 107.

FIG. 108 shows the test results for water including bromide after treatment by an embodiment of the present invention.

FIG. 109 shows the effect of one treatment chamber configuration of the present invention on the treatment of dieldrin.

FIG. 109A shows the effect of one treatment chamber configuration of the present invention on the treatment of dieldrin.

FIG. 109B shows the effect of one treatment chamber configuration of the present invention on the treatment of dieldrin.

FIG. 109C shows the effect of one treatment chamber configuration of the present invention on the treatment of dieldrin.

FIG. 110 shows the test results of treatment of water including dieldrin by one configuration of the present invention.

FIG. 111 shows one set of results of the system of the present invention on water drawn from the Pasig River.

FIG. 111A tabulates one set of results of the system of the present invention on water drawn from the Pasig River.

FIG. 111B tabulates a second set of results of the system of the present invention on water drawn from the Pasig River.

FIG. 112 shows a first set of results in the treatment of raw water in sewage water by an embodiment of the present invention.

FIG. 112A shows a second set of results in the treatment of raw water in sewage water by an embodiment of the present invention.

FIG. 113 shows the results of a natural and synthetic hormone removal by an embodiment of the present invention.

FIG. 114 shows the results of treatment of a first sample with very high level of cadmium, aluminium and zinc by an embodiment of the present invention.

FIG. 114A shows the results of treatment of a second sample with very high level of cadmium, aluminium and zinc by an embodiment of the present invention.

FIG. 115 shows the testing of biodiesel treated by an embodiment of the present invention.

FIG. 116 is a comparison of production of ozone by corona discharge compared to the production method of an embodiment of the present invention.

FIG. 117 is a comparison of ozone generated with the system of the present invention and not functioning and when functioning.

FIG. 118 shows the result of a seawater test treated by an embodiment of the present invention.

FIG. 119 is a photograph of a treatment area of a preferred embodiment viewed through a high-quality camera lens with UV filter.

FIG. 120 is the same chamber illustrated in FIG. 119 viewed by the naked eye showing the energized ozone bubbles.

FIG. 121A is a photograph showing copper and nickel and other metals as suspended solids in a water sample.

FIG. 121B is a photograph showing the copper and nickel precipitated out after treatment from an embodiment of the present invention.

FIG. 122A is a photograph showing iron sulphate as a suspended solid in a mine water sample.

FIG. 122B is a photograph showing iron sulphate precipitated out after treatment from an embodiment of the present invention.

FIG. 123 is an isometric view of a treatment system according to a preferred embodiment.

FIG. 124 is a side elevation view of the treatment system illustrated in FIG. 123.

FIG. 125 is a table showing the results of the treatment of a wastewater stream using the system illustrated in FIG. 123.

FIG. 125A is a table showing the results of the treatment of a different wastewater stream using the system illustrated in FIG. 123.

FIG. 126 is an isometric view of an ozone generator according to a preferred embodiment.

FIG. 127 is an isometric view of a further ozone generator according to a preferred embodiment.

FIG. 128 shows the effect of a treatment chamber configuration illustrated in the “Machine” portion on brine (water with salt added).

FIG. 129 is shows the effect of a treatment chamber configuration illustrated in the “Machine” portion on brine (water with salt added).

FIG. 130 is shows the effect of a treatment chamber configuration illustrated in the “Machine” portion on brine (water with salt added).

FIG. 131 is shows the effect of a treatment chamber configuration illustrated in the “Machine” portion on brine (water with salt added).

### DESCRIPTION OF EMBODIMENTS

According to particularly preferred embodiments of the present invention, a waste treatment system is provided.

The waste treatment system of the invention is illustrated in a wide variety of embodiments in the Figures but all of the embodiments illustrated include at least one treatment chamber through which a fluid to be treated is pumped in a fluid flow, the or each treatment chamber including at least one of:
i. a number of ultraviolet light sources; and
ii. at least one dispersion assembly located at a base of the treatment chamber to disperse at least one sterilizing agent supplied to the treatment chamber in a counter flow direction to the fluid flow.

In the present invention, the ultraviolet (UV) light sources will preferably operate at a wavelength or wavelength band to have a sterilising or disinfecting effect on the fluid at least reducing and preferably eliminating harmful bacteria, viruses and other microbes from the fluid. Additionally, at least some of the ultraviolet light sources will be used to create ozone or ozone enriched air which will be supplied to or formed in a treatment chamber and will have a complementary oxidative effect in the fluid to be treated.

In a one embodiment, one or more UV light source may be provided to have a dual effect, namely to produce ozone and to have a sterilising or disinfecting effect. One or more UV light source may be provided that produce UV light with two main peaks in the UV light band, one at or around 254 nm for a sterilising or disinfecting effect, and another at or around 185 nm for ozone production. However, in a preferred embodiment, all UV light sources within a treatment chamber would have only a sterilising or disinfecting effect (and not an ozone generation effect).

Ozone generation from oxygen exposed to UV light typically occurs at between 160-240 nm. Above 240 nm light will preferably have sterilising or disinfecting effect.

A shortwave, low pressure UV lamp can be used for this purpose. These lamps will typically produce UV light with two main peaks in the UV light band at or around the desired wavelengths.

Importantly, light at wavelengths at or around 254 nm (or generally above approximately 240 nm) will normally adversely affect the levels of ozone in the fluid, normally degrading the ozone by photolysis. Therefore, the light from the ozone generating UV light sources may be located at a different treatment location within the same treatment chamber to the sterilising or disinfecting light sources to maximise the respective effects.

Alternatively, a number of separate treatment chambers may be used, one treatment chamber with the ozone generating UV light sources optimised to emit light waves predominantly at or around 185 nm for ozone production, and an adjacent treatment chamber having sterilising or disinfecting light sources optimised to emit light waves at or around 254 nm for a sterilising or disinfecting effect.

Typically, the ozone generating UV light sources are provided prior to the sterilising or disinfecting light sources in the direction of flow of the fluid through the one or more treatment chambers. This will allow the ozone time to react with the fluid and any constituents before the photolytic destruction of the ozone by the light waves at or around 254 nm for a sterilising or disinfecting effect.

A UV light ozone production system is preferred in the present invention to minimise the production of NO₂ when compared to a corona discharge system in which the N₂ is converted to nitric acid.

In a preferred form, a pre-filtration step may be used. Any type of filter can be used, but a simple sand filter may be optimum in terms of simplicity and low cost.

Preferably, ozone or ozone enriched air may be produced and supplied to the at least one dispersion assembly (primary ozone creation). This is preferably achieved using atmospheric air as a feed material, generating ozone from the oxygen exposed to UV light at between 160-240 nm. The feed pressure for the atmospheric air is preferably low, around 3 psi, as this will allow time for conversion of the oxygen in the feed to be converted to ozone, maximizing the levels of ozone created.

Secondary ozone generation will preferably occur in the treatment chamber using UV light sources optimized to emit light waves predominantly at or around 185 nm for ozone production.

The present invention includes at least one treatment chamber through which a fluid to be treated is pumped. As mentioned above, there will typically be one or more treatment chambers and preferably, a plurality of treatment chambers. Typically, the plurality of treatment chambers is provided in an in-line configuration with the fluid exiting a first treatment chamber entering a second treatment chamber and then exiting the second treatment chamber and entering a third treatment chamber and so on. In one embodiment, the system includes a plurality of treatment chambers provided in an in-line configuration with the fluid to be treated flowing sequentially through the plurality of treatment chambers.

The number of treatment chambers provided will typically be determined according to the efficiency of the treatment chambers and/or any required output conditions and/or based on input conditions of the fluid to be treated.

Typically, a number of treatment chambers will be connected in series although the physical location of the treatment chambers need not necessarily be linear. Where a number of treatment chambers are provided, the treatment chambers are connected to allow flow through the treatment chambers in the treatment system and the physical configuration of the treatment chambers is less important.

Where a plurality of treatment chambers is provided, one or more of the treatment chambers are typically activated in order to treat the fluid to be treated. The number and configuration of treatment chambers which are activated can differ. For example, some treatment chambers may be activated whilst others are deactivated, some or all of the treatment chambers may be partially activated that is some of the UV light sources in some or all of the treatment chambers may be activated and some not activated in order to optimise the treatment of the fluid to be treated. The status of each of the UV light sources in all of the treatment chambers are preferably individually adjustable (on/off and/or the capacity of operation from 0 to 100%) in order to obtain the desired treatment effect. In one embodiment, one or more of the treatment chambers are activated and one or more treatment chambers are deactivated in order to treat the fluid to be treated.

Each treatment chamber may have an internal surface which is at least partially reflective in order to maximise the use of the UV light waves which are produced.

Each treatment chamber will be shaped and preferably cylindrically shaped. However, in some embodiments, the treatment chamber may be rectangular or even multisided such as hexagonal shaped.

In the embodiments where more than one treatment chamber is provided, appropriate connection piping to connect the treatment chambers is also preferably provided. The connection piping may take any physical configuration. For example, in some embodiments, connection piping may be provided to connect an outlet at the base or lower portion of a first treatment chamber with an inlet at an upper position of a second, adjacent treatment chamber. This connection piping may be angled or alternatively, may be formed from a number of portions, for example a first portion which is substantially horizontal with a second portion which is substantially vertical or vice versa.

The flow direction of the fluid to treated through each treatment chamber is in a direction which is opposite to the direction in which the at least one sterilising agent is dispersed into or through the treatment chamber. Generally, the dispersion assembly is located at the base of a treatment chamber and therefore, the fluid will typically be provided into the treatment chamber through an inlet located at an upper portion of the treatment chamber and exit the treatment chamber through an outlet located at or towards the bottom of the treatment chamber.

One or more waste outlets will typically be provided in each treatment chamber. The specific location of a waste outlet in each treatment chamber would generally be dependent upon the type of waste which is to be removed. For example, any waste which may be contained in a froth or similar will typically be removed from an upper portion of the treatment chamber and therefore, the waste outlet will normally be in an upper portion of the treatment chamber. Similarly, any waste in the form of sediment will typically be removed from a lower portion of the treatment container.

Preferably, any treatment container which is designed to have or promote sedimentation will typically not include a dispersion assembly located at the base of the treatment chamber as this would tend to keep the sediment in suspension rather than allowing sedimentation to occur.

A drain valve may be provided in a lower portion of each of the treatment chambers in order to drain fluid from the treatment chamber.

As mentioned above, each treatment chamber will typically be in fluid connection with an adjacent treatment chamber where more than one treatment chamber is provided in series. A single connection may be provided or more than one fluid connection can be provided between linked treatment chambers.

The fluid connection will normally be provided by one or more connector conduits extending between adjacent treatment chambers. Additional treatment components may be provided in association with the connector conduit.

For example, one or more UV light sources may be provided within a connector conduit such that fluid flowing through the connector conduit will flow past of the UV light sources thereby being treated by the one or more UV light sources.

One or more chlorinators or similar components may be provided in association with and/or within a connector conduit such that fluid flowing through the connector conduit can be treated with chlorine produced by the one or more chlorinators.

Each treatment chamber will typically be provided with a top wall and a bottom wall and at least one side wall extending between the top wall in the bottom wall. Preferably, the top wall in bottom wall are provided as or with a removable top and/or bottom to provide or allow access to an internal part of each of the treatment chambers.

The treatment system of the present invention may be provided within a housing to allow easy transport. In particular, a treatment system according to the present invention may be provided within one or more shipping containers, or in or in association with a floating vessel or boat designed to travel across a body of fluid to be treated. If ancillary equipment such as filters or pumps and the like are provided, these can be provided in the same housing as the treatment system or alternatively, one or more additional housings may be provided to house ancillary equipment which can then be associated with the housing which houses the treatment system. In one embodiment, the treatment system is mounted on a floating vessel or boat designed to move across a body of fluid to be treated with the movement of the floating vessel or boat causing flow through the treatment system. In one embodiment, the treatment system mounted on a floating vessel or boat is mounted on a moveable portion to be moved between a raised, non-active position and a lowered used position.

The treatment system of the present invention includes a number of ultraviolet light sources provided within at least one treatment chamber. The ultraviolet light sources can be provided in the same treatment chamber as the at least one dispersion apparatus or ultraviolet light source may be provided in one or more treatment chambers which are separate from one or more treatment chambers housing the at least one dispersion apparatus.

Preferably, a number of ultraviolet light sources are provided in a number of different treatment chambers. As mentioned above, a treatment chamber may be provided with a number of ultraviolet light sources with or without a dispersion assembly. Preferably, the light sources will be configured to provide different wavelengths of light to the fluid to be treated. The light sources may treat the fluid directly and/or create secondary materials such as ozone for example that in turn treat the fluid, typically by acting on the materials carried by the fluid which the treatment is designed to remove.

According to a particularly preferred embodiment, the ultraviolet light sources will provide light waves at at least approximately 180 nm and approximately 254 nm. Without wishing to be limited by theory, light sources at approximately 180 nm will preferably create ozone and light sources at approximately 254 nm will preferably treat biologics for example.

Light may be provided many wavelengths including these in particular.

Light waves which are introduced into the fluid at one wavelength may also be split into light waves of lesser wavelength for example through interactions with materials carried by the fluid to be treated and/or bubbles for example which introduced into the fluid to be treated, for example by the at least one dispersion apparatus.

The UV light sources may be provided in any configuration. For example, as mentioned above, a number of ultraviolet light sources may be provided in a first treatment chamber and then an adjacent treatment chamber may contain a dispersion assembly without UV light sources and/or light sources may be provided in all treatment chambers in a treatment system.

The UV light sources may be grouped within a treatment chamber. For example, the light sources may be physically grouped within a treatment chamber in a bunched configuration in order to enhance the treatment of the fluid. The light sources may be grouped within a treatment chamber or in different treatment chambers according to a particular wavelength which is desired in an area of a treatment chamber or in a treatment chamber. For example, a number of light sources emitting light waves at or around 180 nm may be grouped in a particular location in a treatment chamber with a number of light sources emitting light waves at or around 254 nm grouped in a particular location that is different or separated from the grouping at or around 180 nm.

As mentioned above, any material or bodies within the fluid to be treated may further disperse or divide the UV light such as for example the UV light wavelengths may be split by bubbles from the dispersion apparatus and/or the bubbles may spread the light through the fluid to be treated.

The UV light sources may have any orientation relative to the treatment chamber but will typically be either a vertically oriented or horizontally oriented. UV light sources of a combination of orientations may be provided in a single treatment chamber or each treatment chamber may be limited to UV light sources of a particular orientation.

The UV light sources may be spaced approximately equal across the volume of the treatment chamber in order to ensure treatment and/or grouped as outlined above.

The housing may be provided which is divided using one or more divider panels in order to produce a optimal flow of fluid through the housing. For example, by providing a rectangular housing with a series of divider panels with each alternate divider panel spaced from the floor and each other alternate divider panel attached to the floor but spaced from the top of the body of fluid, the divider panels can cause an under or over flow pattern through the housing.

Where divider panels are provided, the divider panels may be removable and/or movable within the housing. It is preferred that any divider panels are planar but any shape could be used.

Typically, pipework will be provided relative to a housing that is provided with divider panels in order to assist with the flow of either the fluid to be treated or other fluids which are provided to the treatment chamber such as for example a least one sterilising agent.

At least one treatment chamber in the treatment system of the present invention will typically include at least one dispersion assembly located at a base of one or more treatment chambers in order to disperse at least one sterilising agent supplied to the treatment chamber through the fluid to be treated. In a particularly preferred form, the sterilising agent will be ozone. In this way, ozone or gas charged with ozone can be provided to a treatment chamber in order to treat fluid, typically by oxidation of components or material being carried by the fluid to be treated.

At least one dispersion assembly may be provided in each treatment chamber within the system or only in one or more treatment chambers. In a particularly preferred embodiment, a number of treatment chambers are provided with each alternating treatment chamber provided with a number of ultraviolet light sources and each other alternating treatment chamber provided with at least one dispersion assembly. However, in some embodiments, a dispersion assembly can be provided in every treatment chamber with the UV light sources provided in only some, preferably alternating treatment chambers within the system.

In some configurations, a treatment chamber will have ozone created in situ within the treatment chamber through the provision of UV light sources within the treatment chamber and at least one dispersion assembly will add ozone or air enriched with ozone to the same treatment chamber in order to enhance treatment.

Additional treatment chambers may be provided without any ultraviolet light sources or dispersion assemblies such as for example to allow sedimentation of material which has been treated by the UV light sources and/or dispersion assembly to settle to allow removal from the fluid to be treated.

The means for introducing the sterilizing agent into the primary treatment chamber may comprise one or more gas outlets, the one or more gas outlets comprising one or more of air stones, a gas permeable pipe or pipes, a diffuser or diffusers or an external venturi or venturis communicating with the primary treatment chamber and a source of the sterilizing agent.

Preferably, the sterilizing agent is introduced in the form of bubbles.

The means at the upper end of the chamber for removing waste may comprise an inverted U-shaped trap and/or a venturi unit.

Preferably, the system may further compromise a means at the upper end of any one or more of the treatment chambers for removing waste in the liquid conveyed by the bubbles upwardly through the chamber. The means at the upper end of the chamber for removing waste may compromise an outlet, preferably located above the preferred upper inlet for fluid to be treated preferably to take advantage of any flotation of waste materials, especially particulate or agglomerated material that may be carried upward by the bubbles and in particular, any waste materials in froth that may be formed. Each treatment chamber will preferably have a waste outlet and preferably, the respective waste outlets are associated with a common waste carriage conduit. In a preferred form, the common waste carriage conduit may have air or a gas pumped therethrough to create a suction effect at the waste outlet to assist with extraction of the waste materials.

A tube or similar may be used in association with any one or more of the plurality of UV light sources. The UV light sources are preferably elongate and/or are provided in an elongate tube to allow the light to pass and/or to disperse the light. Preferably, the tube will be or include a quartz tube. The preferred quartz tube can be inserted into the treatment chamber horizontally or vertically, may extend across the width of the chamber or vertically substantially over the height of the treatment chamber. Preferably, a quartz tube or similar may extend substantially the length of the UV light source and the tube preferably protects the ultraviolet light from the fluid in the treatment chamber.

Preferably, a quartz tube or similar may extend substantially the length of the connection conduit and the tube protects the ultraviolet light from the fluid in the connection conduit.

Preferably, the tube may be manufactured from clear quartz or from any like materials; the tube allows the light wave from the ultraviolet light to interact with the ozone bubbles and reflect and react around the chamber.

The UV light sources may also be or include LED lights.

Preferably, the system may further comprise an end cap which is releasable attached to the top of each treatment chamber.

The tubes are preferably mounted through a sidewall and/or end cap and/or bottom mall depending upon their orientation.

Preferably, the waste removing means of each treatment chamber is connected to one or more waste pipes that is common. The lower ends of the treatment chambers may be selectively connectable to one or more common drainage pipes or ducts via control valves to allow drainage of the chambers.

Preferably, the system may further comprise at least one pre-filter located in the inlet line before the first chamber for removing any large materials from the fluid to be treated.

Preferably, the system may further comprise a U-shaped bend or trap to retain a level of fluid in the or each waste outlet to prevent any gas, particularly ozone gas or air charged with ozone gas, leaking into the outlet or waste.

Preferably, a combination of different wavelengths lamps may be used in the ozone production to produce ozone gas.

The system preferably operates at fluid to be treated feed pressure below 5 psi. The treatment system of the invention may employ one or multiple treatment chambers.

Treatment chambers can be used to treat fluid to be treated in particular, generally liquid or wastewater will be treated in order to provide clean water as a result.

The additional ozone or air enriched with ozone which may be used according to the present invention is typically created on-site, generally close to the injection location of the ozone or air enriched with ozone. According to a preferred embodiment, the ozone or air enriched with ozone is generally formed using UV light sources as well.

In order to be clear, in a preferred embodiment, typically air enriched with ozone is preferably created by passing atmospheric air passed UV light source is configured to emit light waves at one or more wavelengths conducive to the creation of ozone and then this enriched with ozone is then preferably passed to at least one dispersion assembly located in a treatment chamber to treat liquid.

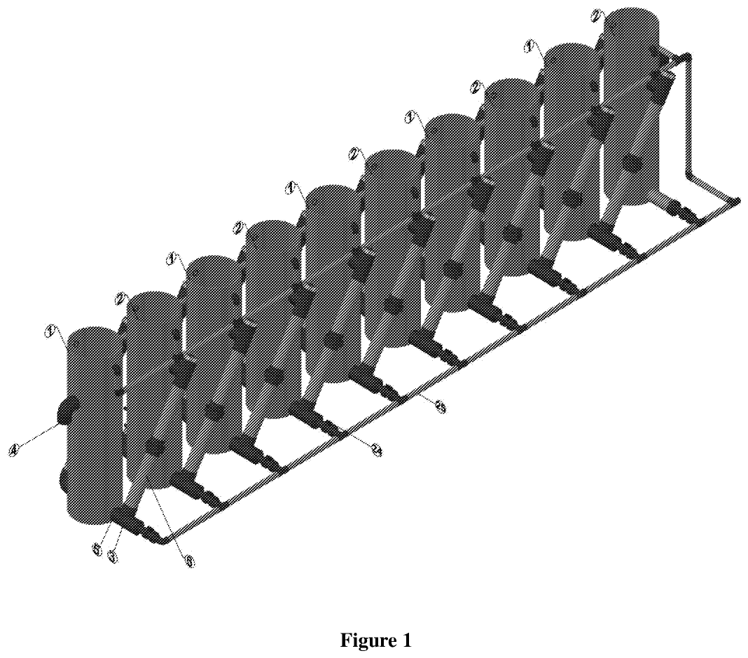

FIG. 1 shows a system of a preferred embodiment with multiple treatment chambers and angled water transfer conduits between them. The treatment chambers 1 contain UV light sources to form ozone in the water to be treated through the application of light waves of a wavelength of approximately 180 nm and treatment chambers 2 include both UV light sources, preferably ozone generating UV light sources optimised to emit light waves predominantly at or around 185 nm for ozone production, sterilising or disinfecting light sources optimised to emit light waves at or around 254 nm for a sterilising or disinfecting effect. An inlet 4 is provided into the first treatment chamber and then subsequent transfer is via an outlet to the angled transfer conduit 6 to the upper inlet of the adjacent treatment chamber. A lower drain outlet is provided for each treatment chamber connected to a common drain pipes 25.

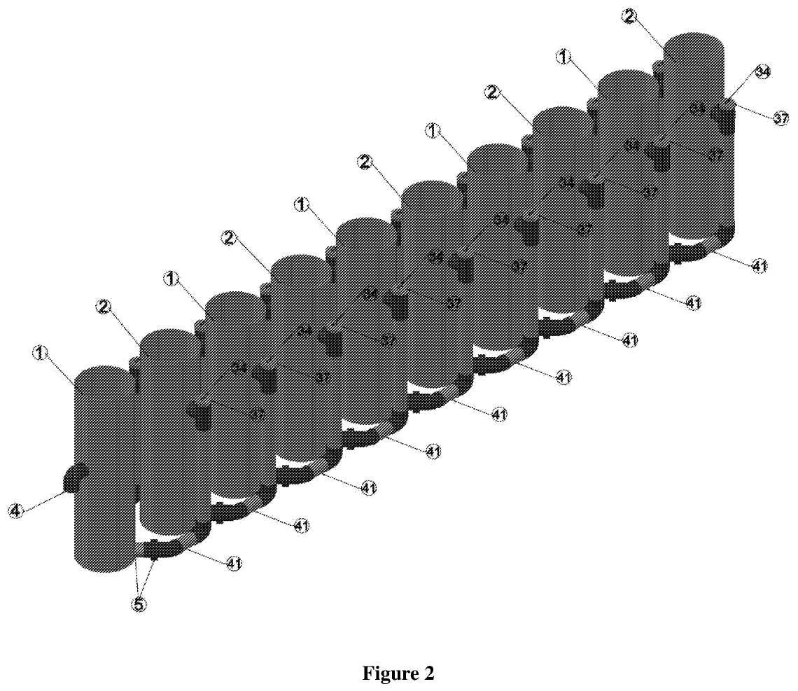

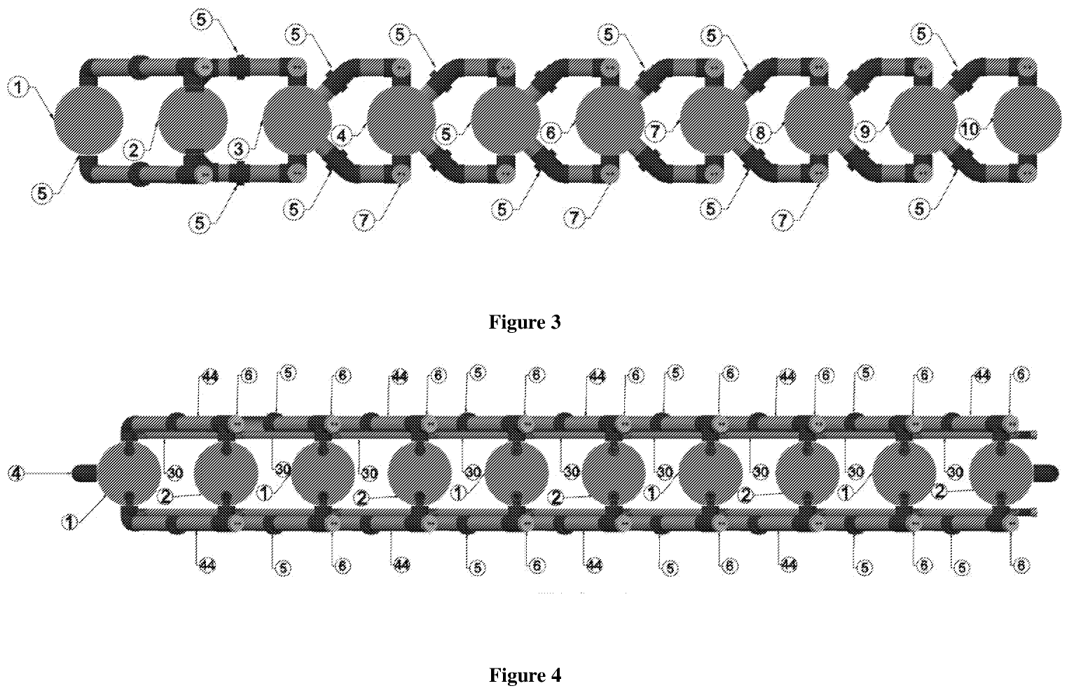

FIGS. 2 and 3 shows an isometric view and top view respectively of a system of multiple treatment chambers in a similar layout to that shown in FIG. 1 but with the curved, horizontal and vertical transfer pipes 41 connecting adjacent treatment chambers.

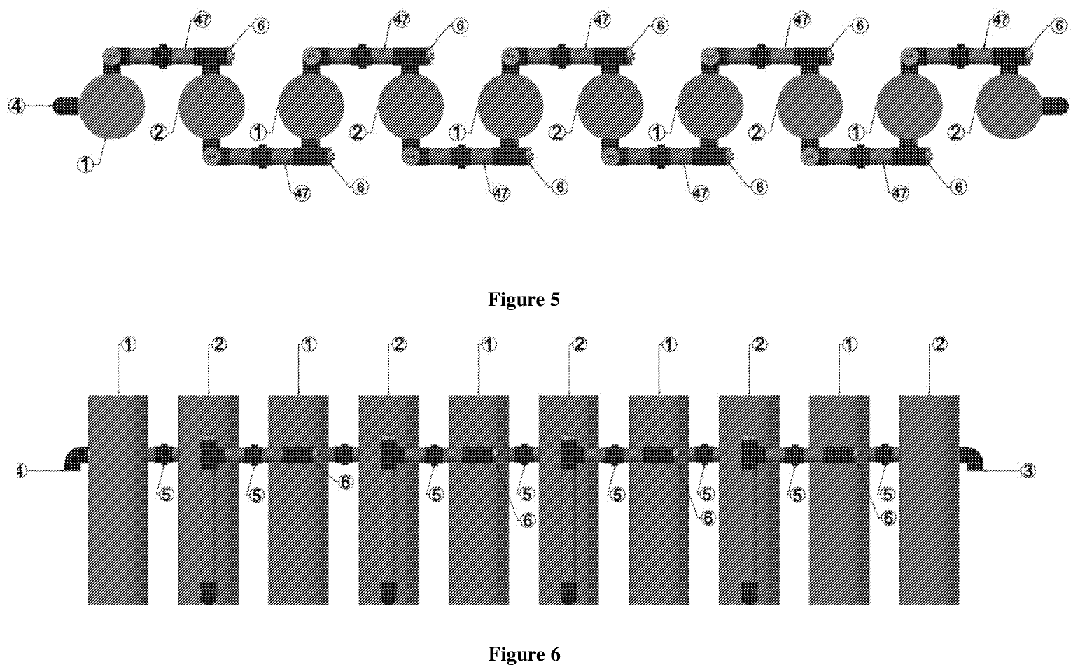

FIG. 4 shows a top view of the configuration illustrated in FIG. 1.

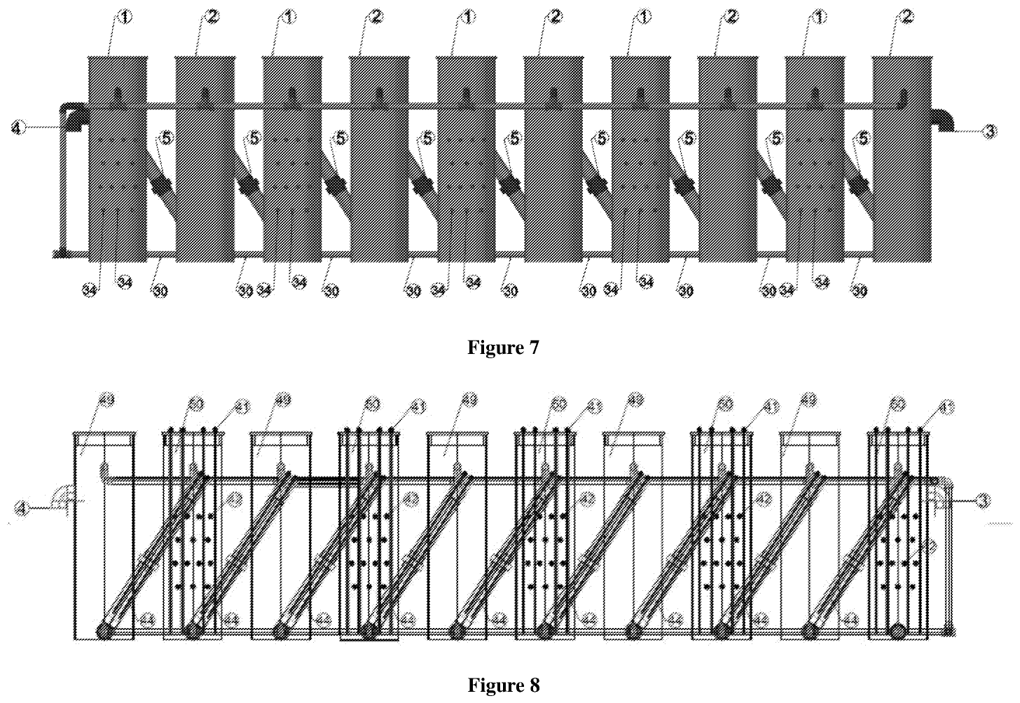

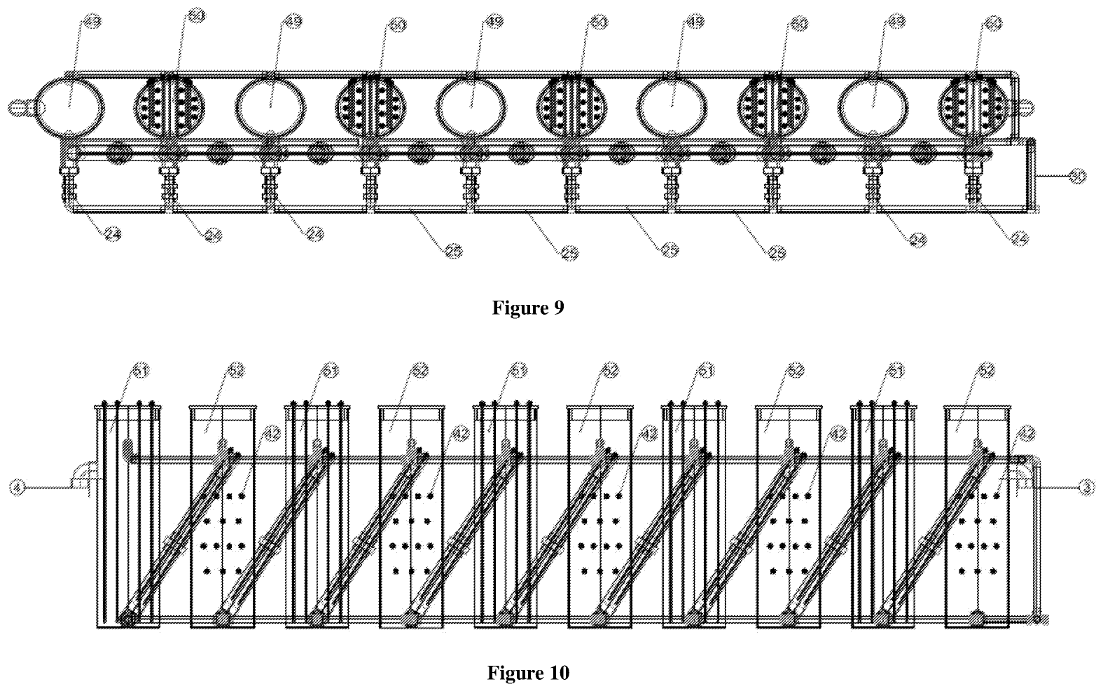

FIG. 5 is a top view similar to that shown in FIGS. 3 and 4 but with alternating vertical-horizontal connection conduits 47. FIG. 6 is a front view showing the vertical-horizontal advance oxidation transfer pipes on alternate sides.

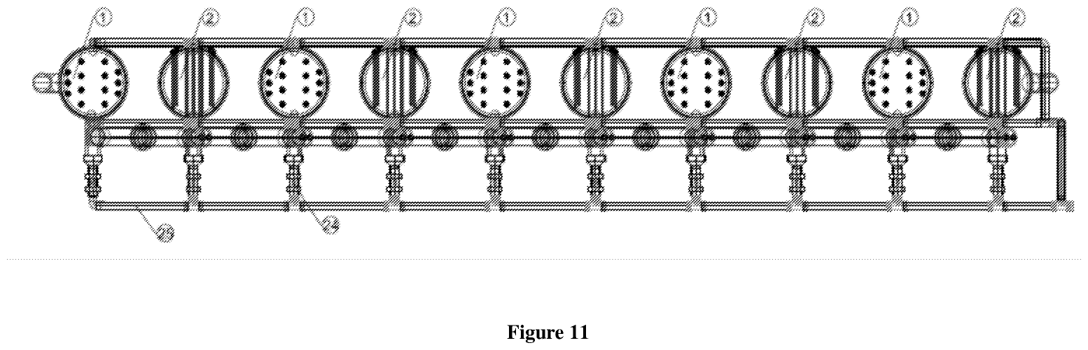

FIG. 7 shows a rear side of a system similar to that shown in FIG. 1 but with connection conduits on only a forward side and showing the ultraviolet bulbs 34 and second fractionation pipe or upper waste outlets which are linked to a common conduit or pipe.

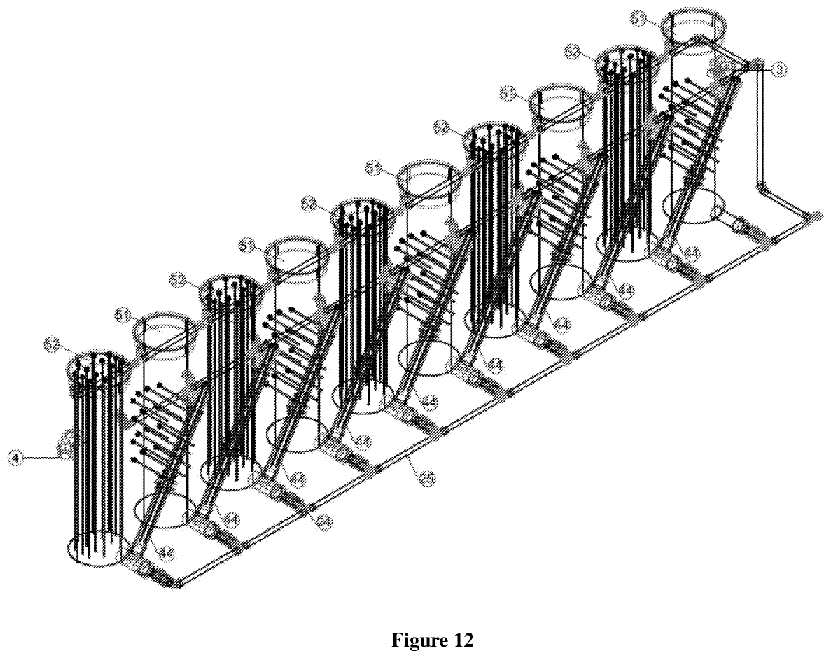

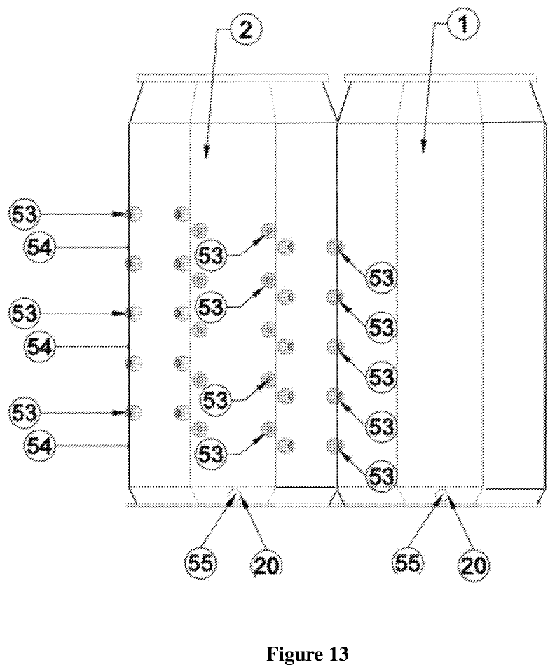

FIG. 8 shows a front view of a system similar in most respects to the earlier embodiments but showing vertical and horizontal UV light sources provided in quartz tubes in every second treatment chamber. Also illustrated is a number of UV light sources provided in a quartz tube concentrically within each connection conduit allowing fluid to flow past the UV light sources provided within each connection conduit for additional treatment. FIG. 9 is a top view of this configuration.

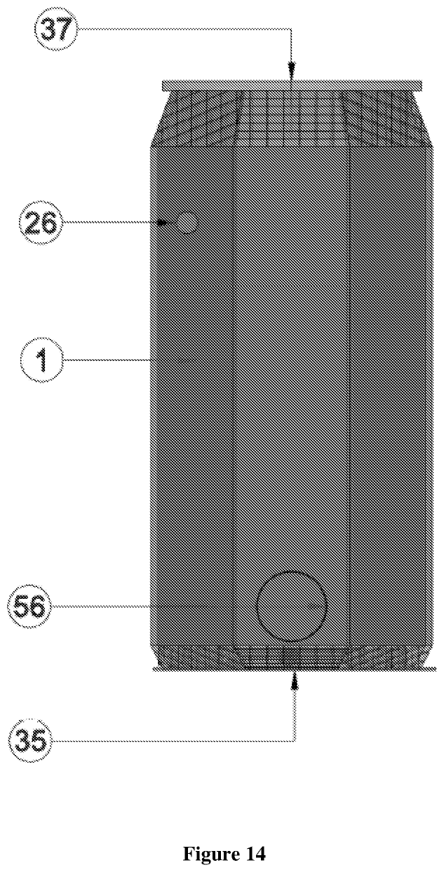

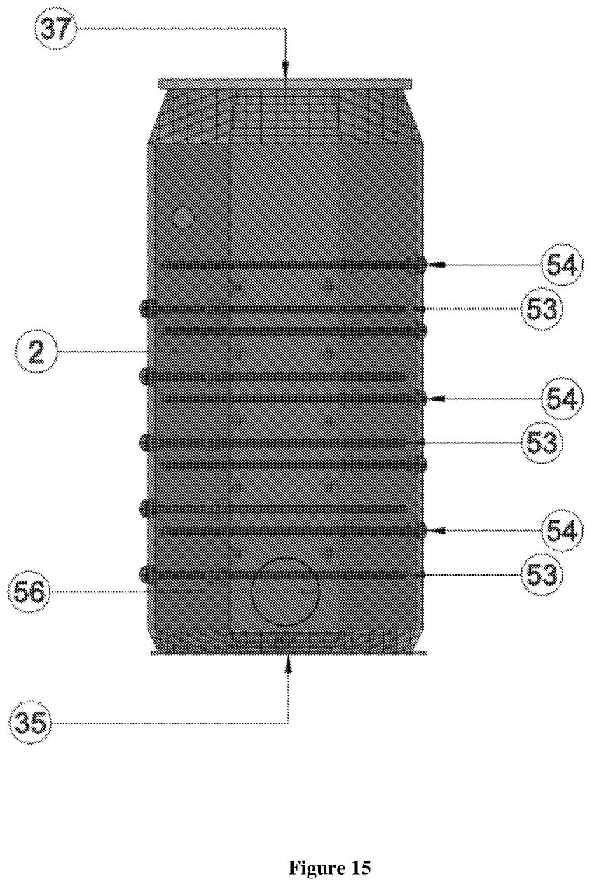

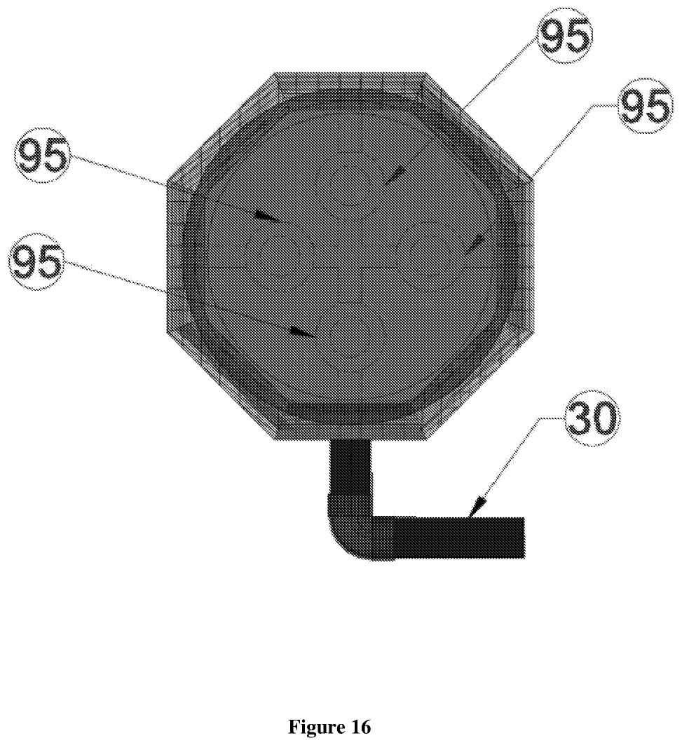

FIG. 10 is a side view of a further alternative configuration showing treatment chambers fitted with UV light sources provided in a quartz tube oriented vertically and UV light sources provided in a quartz tube oriented horizontally in alternating treatment chambers together with a number of UV light sources provided in a quartz tube concentrically within each connection conduit. FIG. 11 is a top view of this configuration and FIG. 12 is an isometric view.

Importantly, the fluid to be treated flows through the treatment chambers of all of these embodiments from an upper end towards a lower end and then up to an elevated inlet of the adjacent treatment chamber and so on.

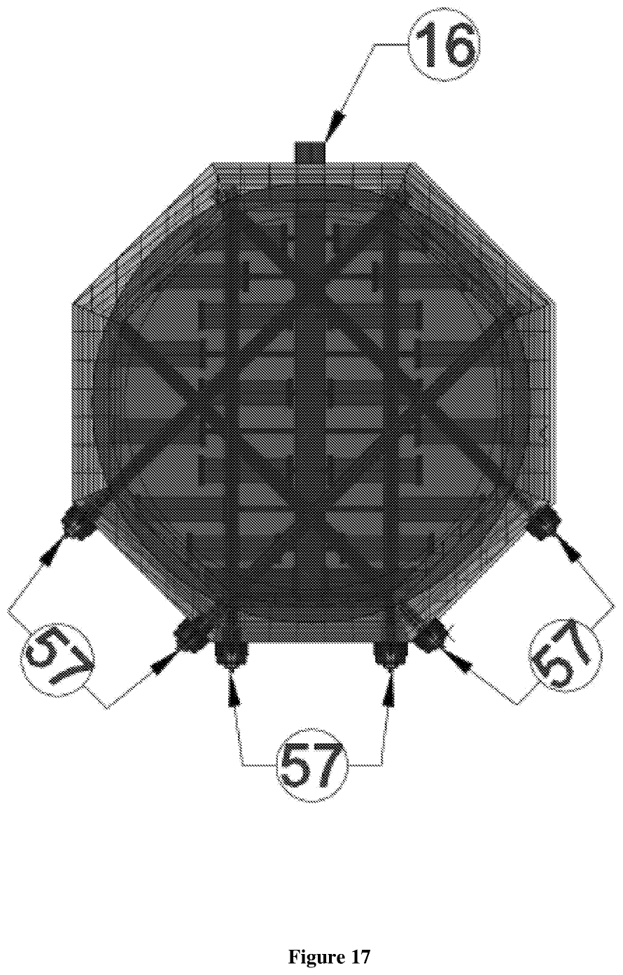

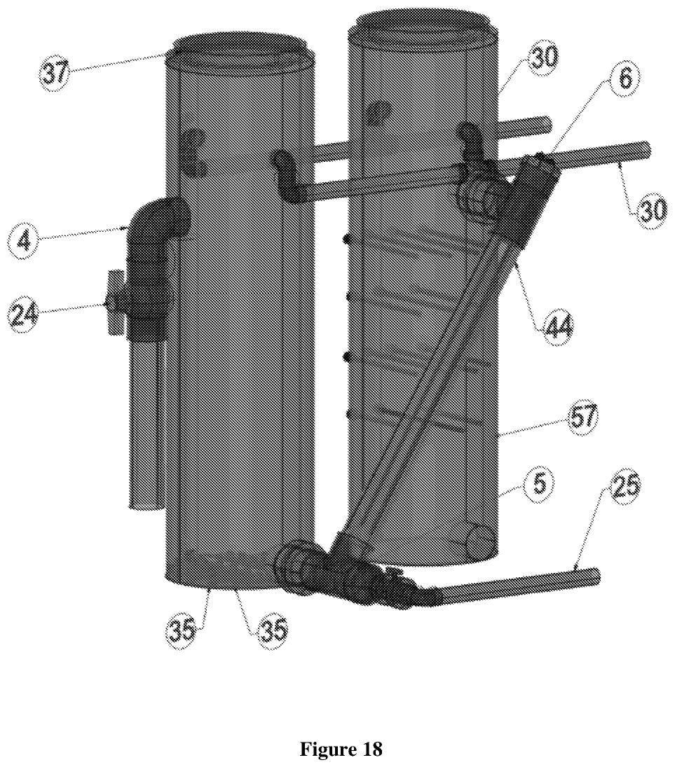

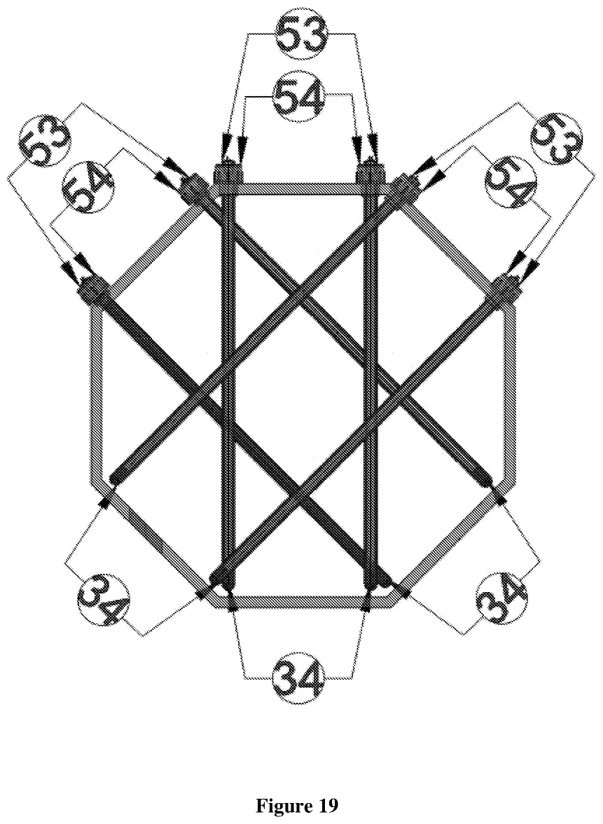

FIG. 13 is a side view of two hexagonal treatment tanks in a side by side configuration and showing the mounting locations for a number of UV light sources in each of three sides of the left side treatment tanks to produce ozone in situ and/or to sterilise the fluid to be treated using approximately 254 nm light waves. The right-side treatment tank is configured as a settling tank. FIG. 14 is a side view of the right-side tank from FIG. 13 and FIG. 15 is a side view of the left side tank from FIG. 13 showing the UV lights extending laterally across the internal volume of the tank.

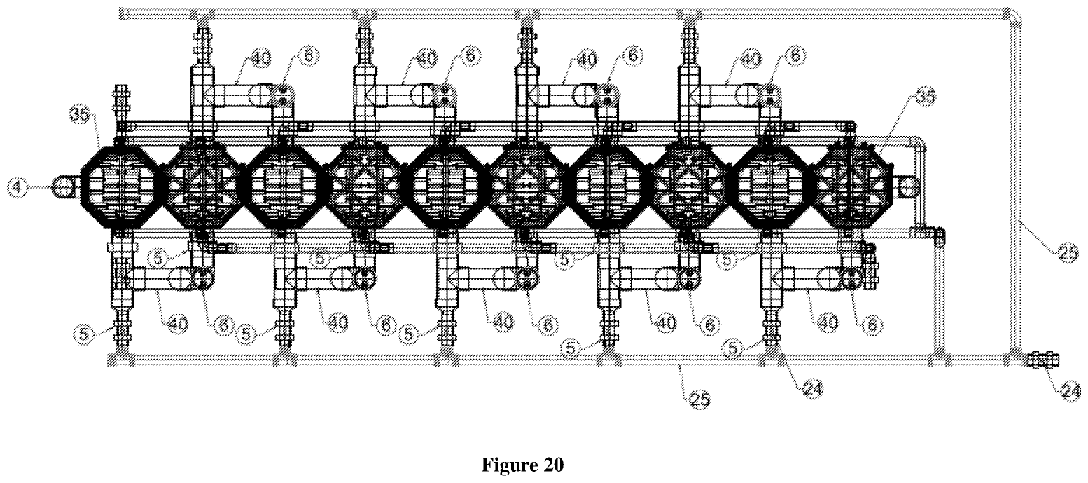

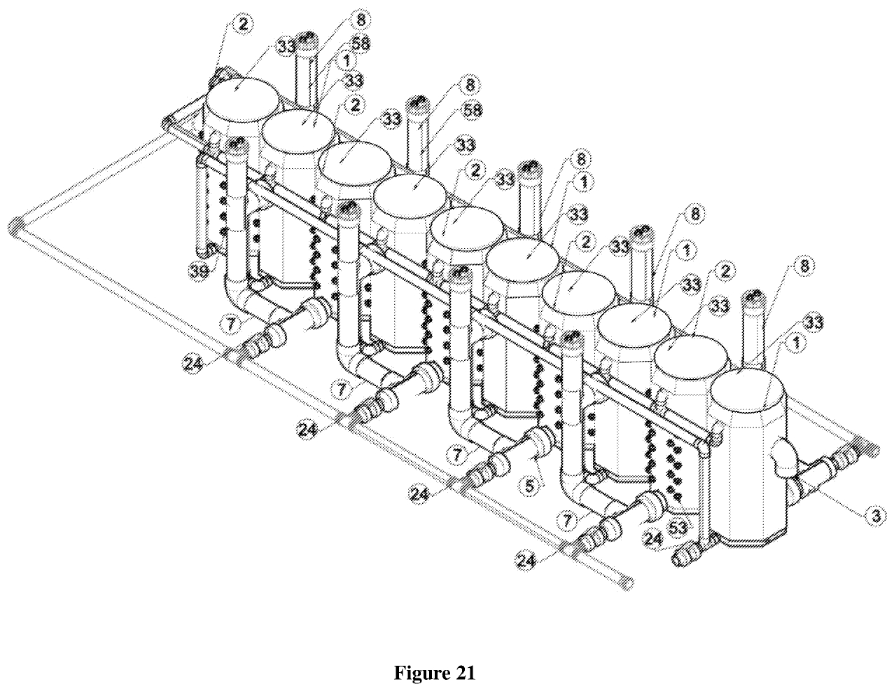

FIG. 16 is a top view of the embodiment shown in FIG. 14 showing the settlement drainage chambers 95 at a lower end of the tank. FIG. 17 is a top view of the embodiment shown in FIG. 15 a diffuser assembly in the base of the treatment tank and the array of UV lights extending laterally across the internal volume of the tank. These UV lights do not have quartz tubes surrounding them.

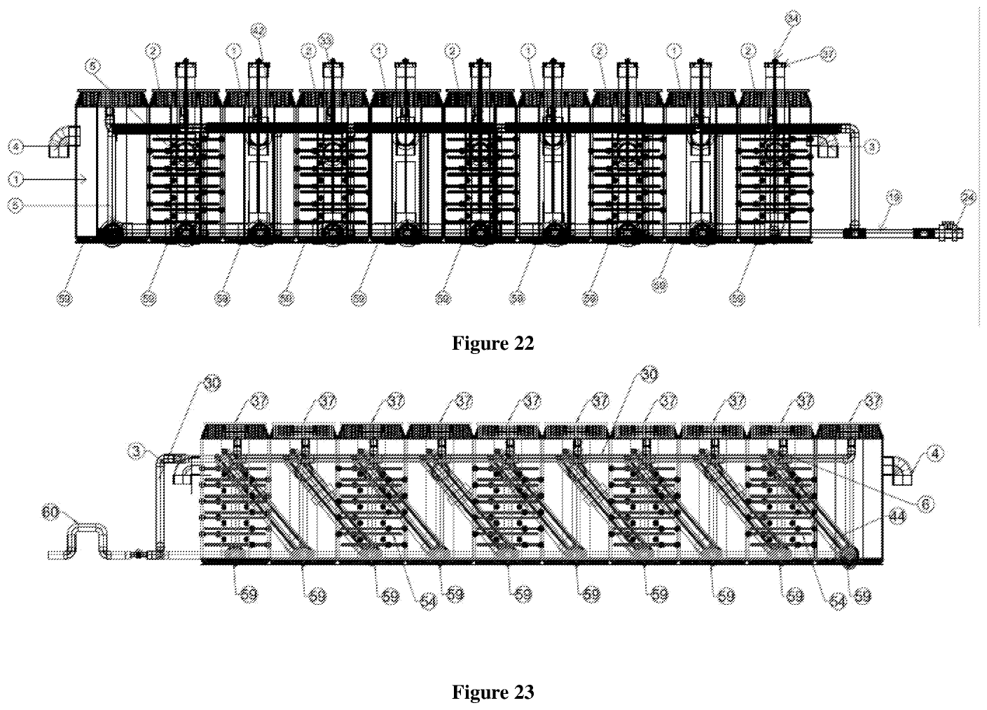

FIG. 18 is an isometric view of a preferred two-tank processing embodiment with connections. The left-side tank includes a diffusion assembly in the form of air stones 35 at the base, which can deliver ozone or air that will rise up through the tank. Also shown are the laterally oriented UV lights in the right-side tank and in the connection conduit between the tanks. Inlet pipe 4 is included as is a lower waste pipe 25 for sediment and an upper waster pipe 30 to remove any floating waste or waste that may be in froth formed.

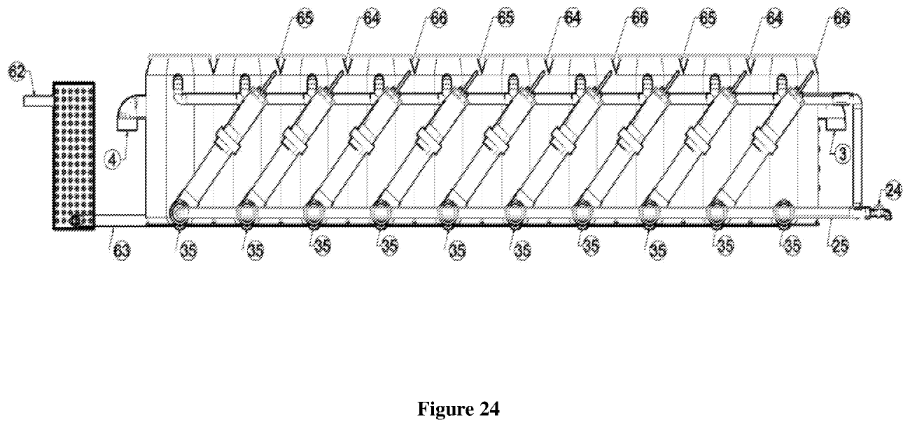

FIG. 19 is a top view of a main embodiment in an octagon shape showing one of a number of layers of multiple UV bulbs in each layer. There will typically more than one layer and the layers can be aligned or partially rotated to be offset from one another to provide enhanced treatment.

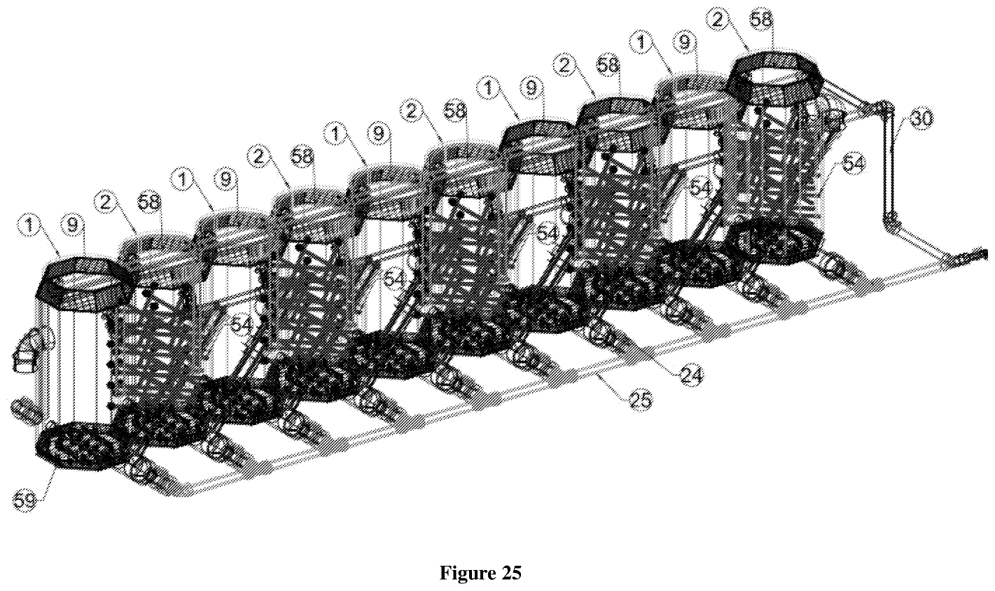

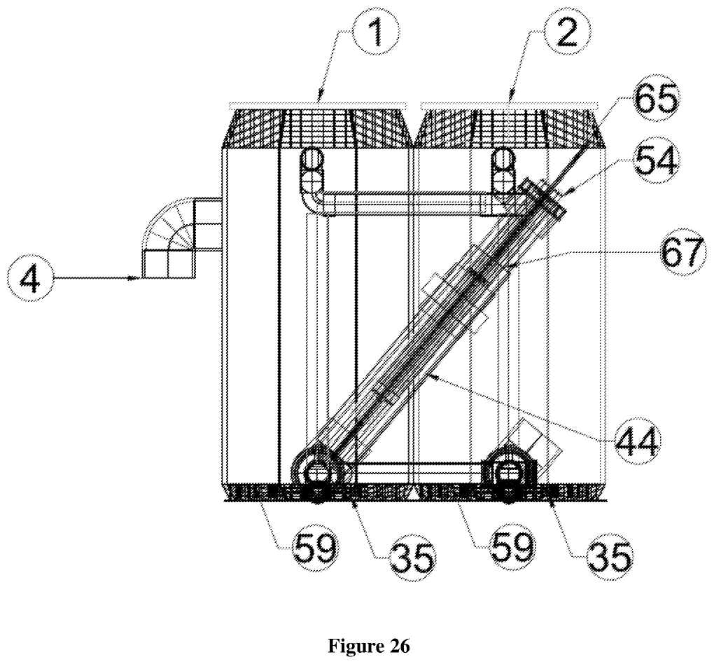

FIG. 20 is a top view is an embodiment showing the horizontal-vertical advance oxidation transfer pipe on the alternating side of each tank two tank processing embodiment from FIG. 18. Also shown is the ozone air stone diffusion assemblies in each tank and the alternating tanks provided with arrays of UV lights as shown ion FIG. 19 together with drain pipes 25 of both sides of the tanks. FIG. 21 is an isometric view of this configuration.

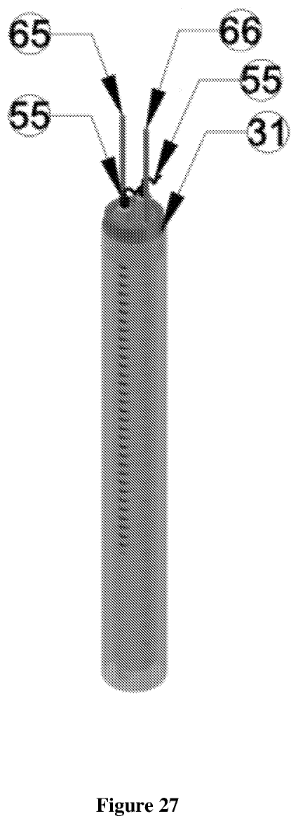

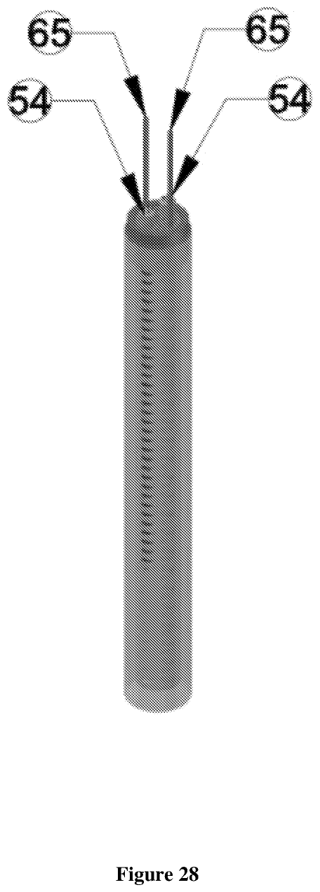

FIG. 22 is a side view of the horizontal-vertical connection conduit between chambers and FIG. 23 is a side view showing an alternative embodiment.

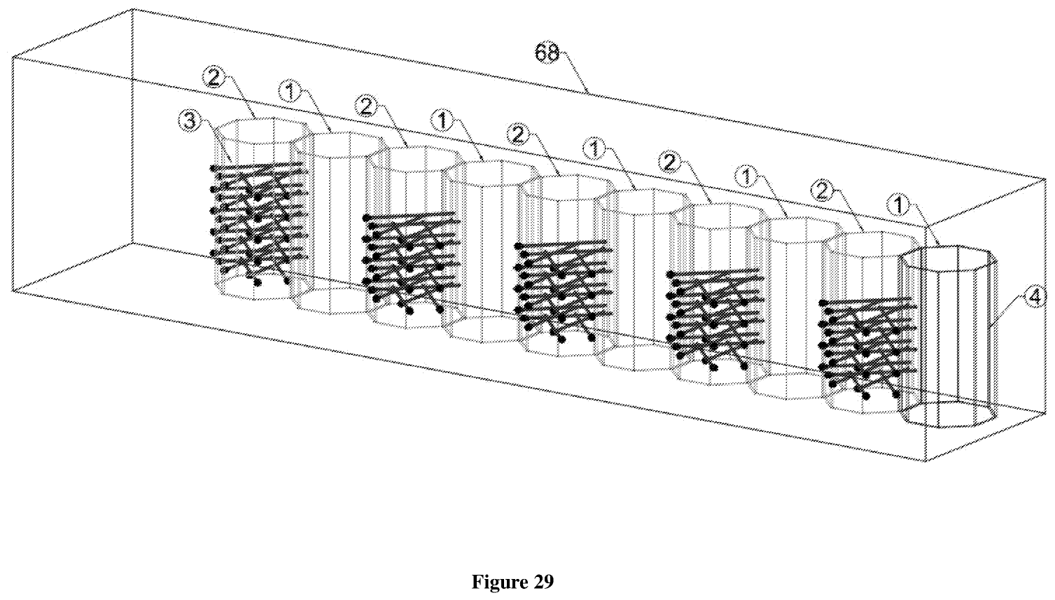

FIG. 24 is a side view of the angled connection conduit embodiment with an associated gas destruction module included on a gas line.

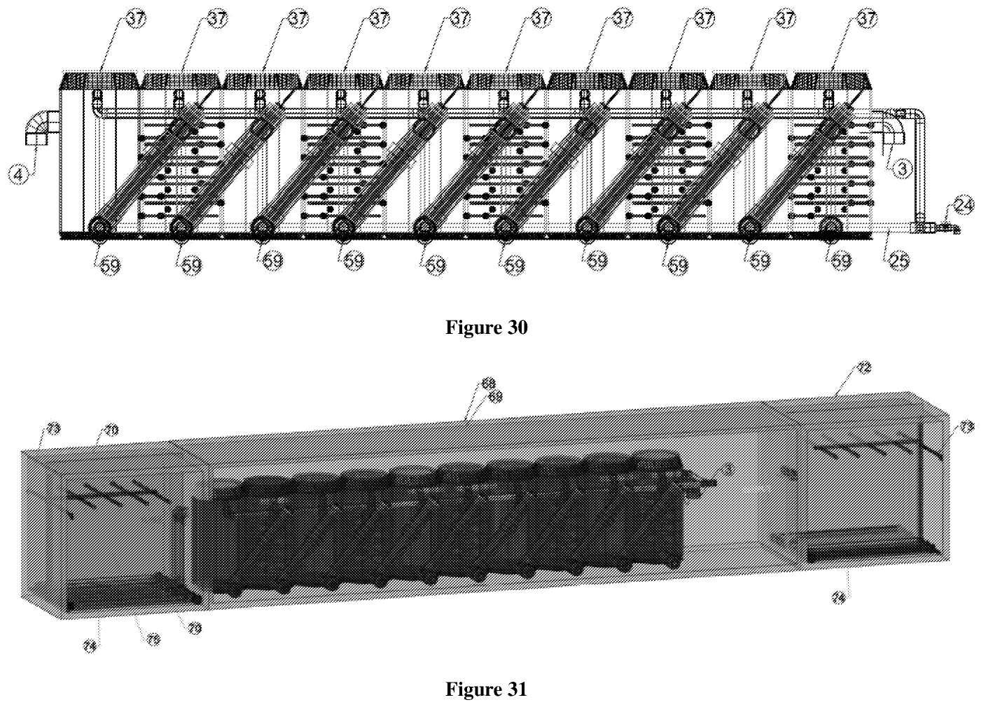

FIG. 25 is a partially transparent isometric view of a preferred system showing internal components of the treatment tanks showing the UV lights oriented at an angle across the tanks rather than horizontally or vertically. In this embodiment, each treatment tank is provided with an ozone diffusion assembly to introduce ozone or ozone enriched air bubbles.

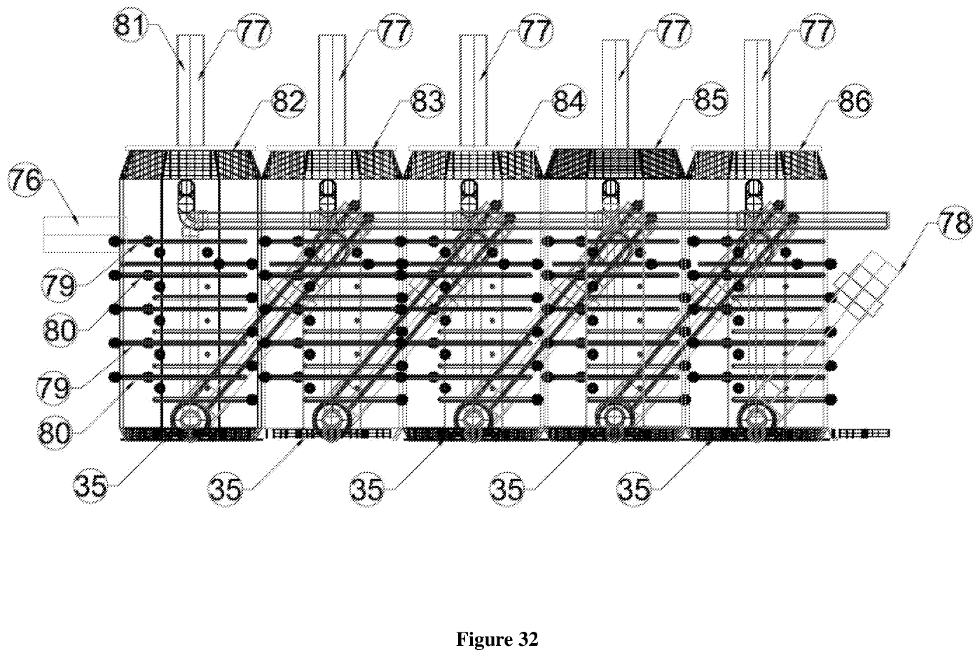

FIG. 26 is a side view of a two-treatment tank embodiment with inlet pipe 4 into a first treatment tank, an angled connection conduit 44 fitted with a chlorinator 67 and at least one UV light.

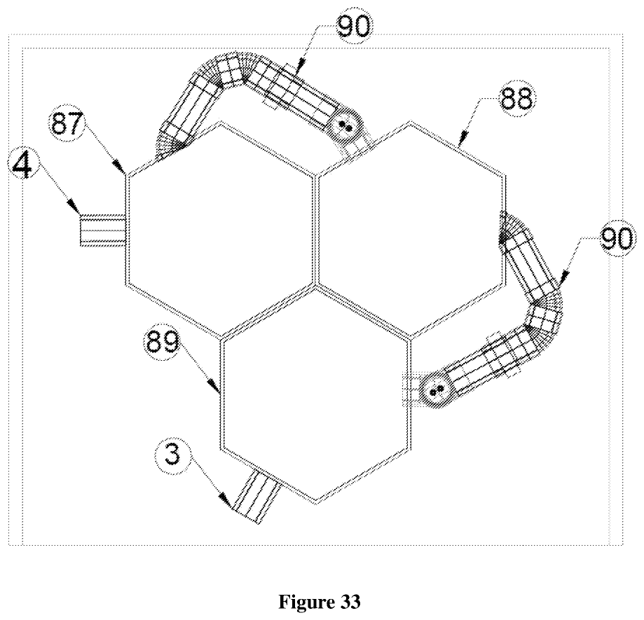

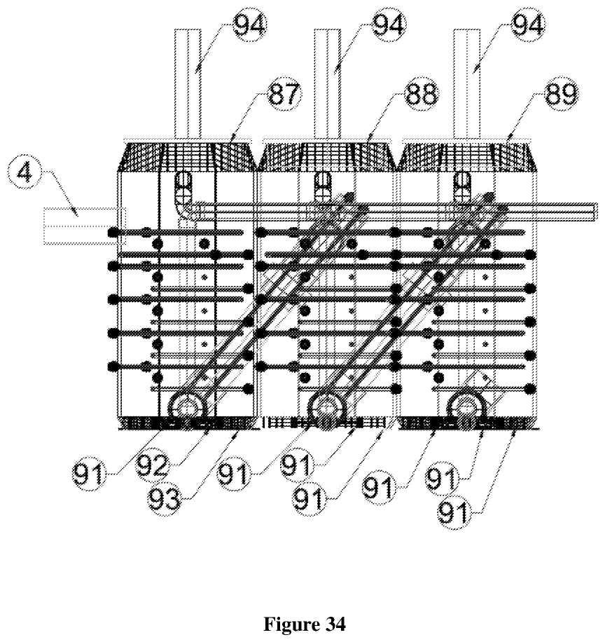

FIG. 27 is a front elevation view of a chlorinator with ultraviolet bulbs and ozone inlets 55. FIG. 28 is a front elevation view of the chlorinator of FIG. 27 with the UV bulbs removed.

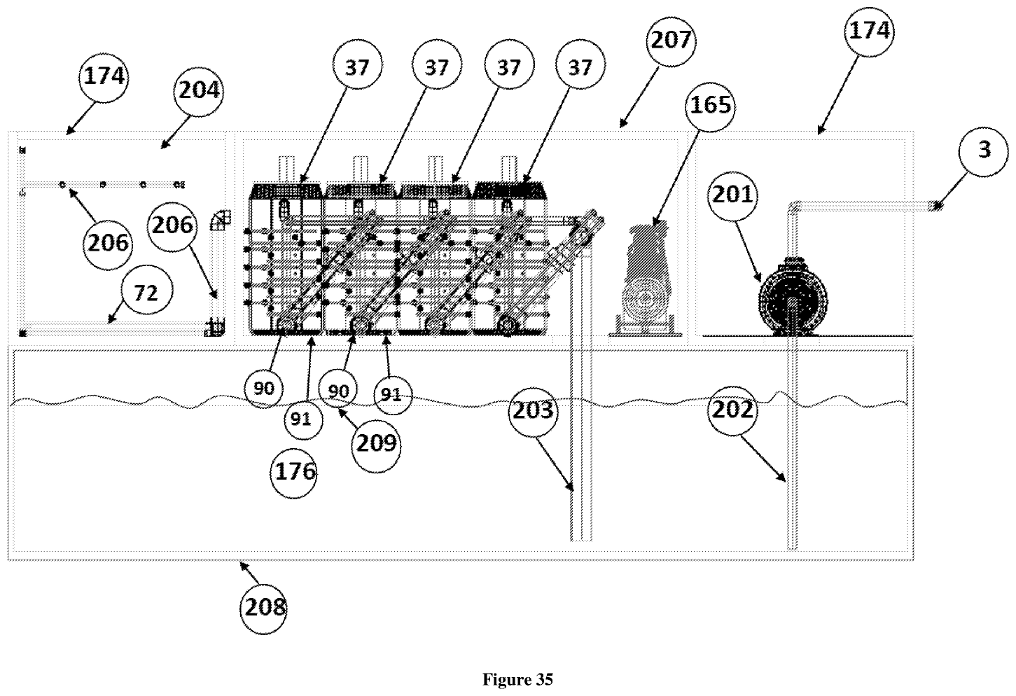

FIG. 29 shows a preferred treatment system fitted with treatment chambers of alternating types fitted into a shipping container 68 as a housing for transport permanent housing and easy transport.

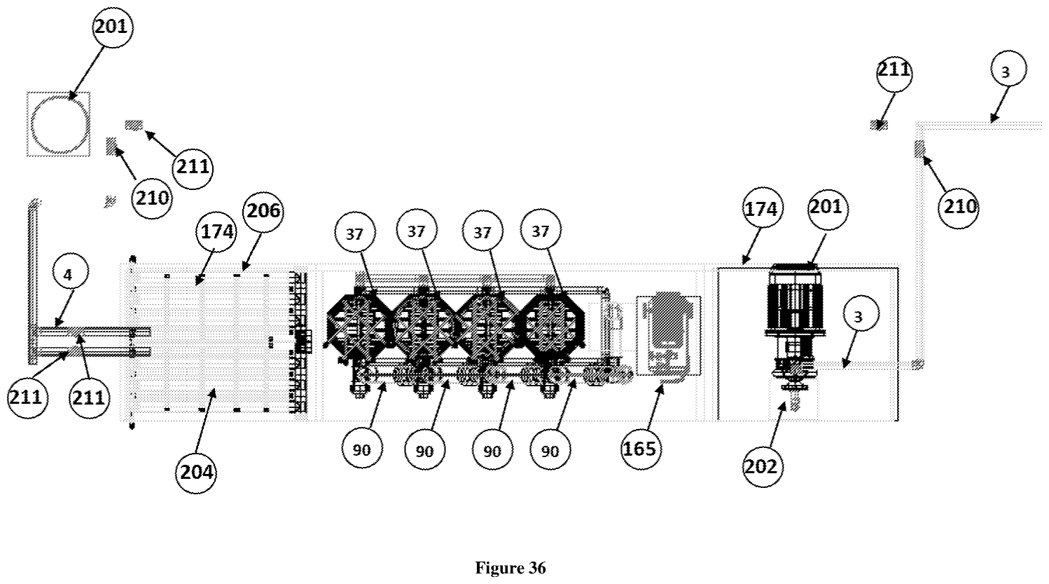

FIG. 30 is a side view of a ten-tank, 5-pair treatment system similar to that shown schematically in FIG. 29 with UV light containing angled connection conduits between adjacent treatment tanks and horizontal UV lights provided in every second tank.

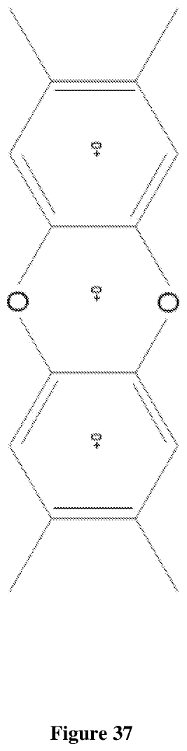

FIG. 31 is a schematic view of a 40-foot shipping container with a filter shipping container at either end for pre-filtering and post filtering.

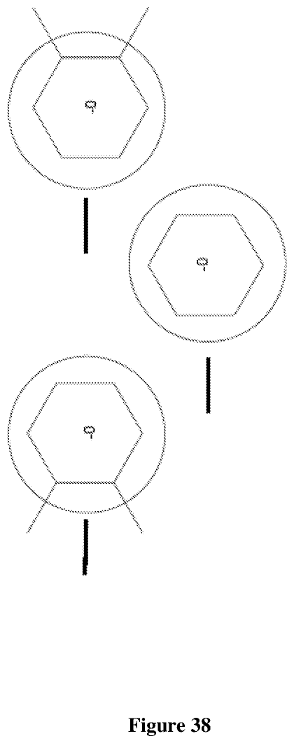

FIG. 32 shows a preferred embodiment of a treatment system in gas destruction or air purification format. Chamber 82 shows a mixture of UV light sources including 3 rows of UB lights at 180 nanometers producing ozone from the oxygen in the air and possible oxygen from the gases. The following 3 rows of ultraviolet tubes are at 253 nanometers or similar to destroy toxin or impurities within the gases. Although illustrated in bands, the UV lights may be provided in alternating layers or lights of different wavelengths can be in the same layer although this is less preferred due to the photolytic effect of 253 nanometer light on the ozone. Chamber 83 is provided with only 180-nanometer tubes producing a massive amount of ozone which is mixing with gases or air or oxygen which will pass to the chamber and chamber 84 has only 253 nanometer UV lights. Chamber 85 is only 180 nanometer UV lights to produce additional ozone. The top half of the UV tubes in chamber 85 are 180-nanometer producing ozone before passing over tubes of the bottom half of the chamber 85 which are at 253 nanometers. Gases passing over the transfer tube of the chamber 86 will then enter a chamber which can be full of varying wavelengths of 0 nanometers up to thousands of nanometers or higher to destroy any remaining gas or germs in the system.

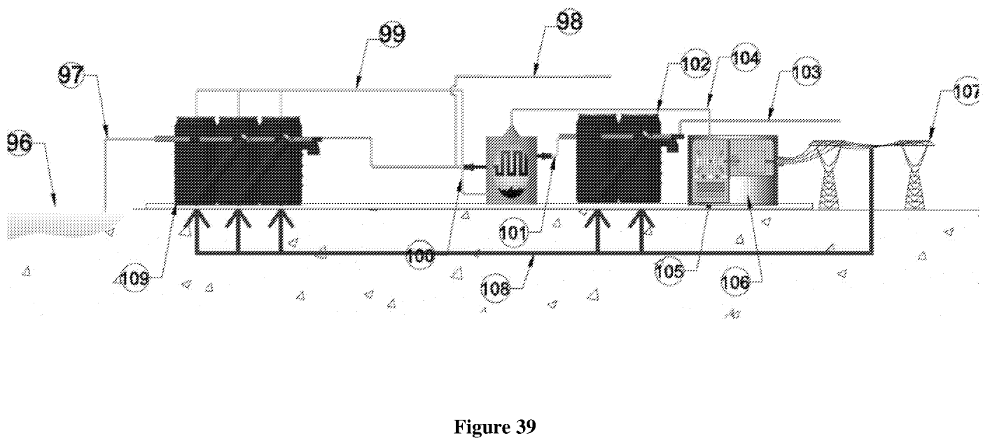

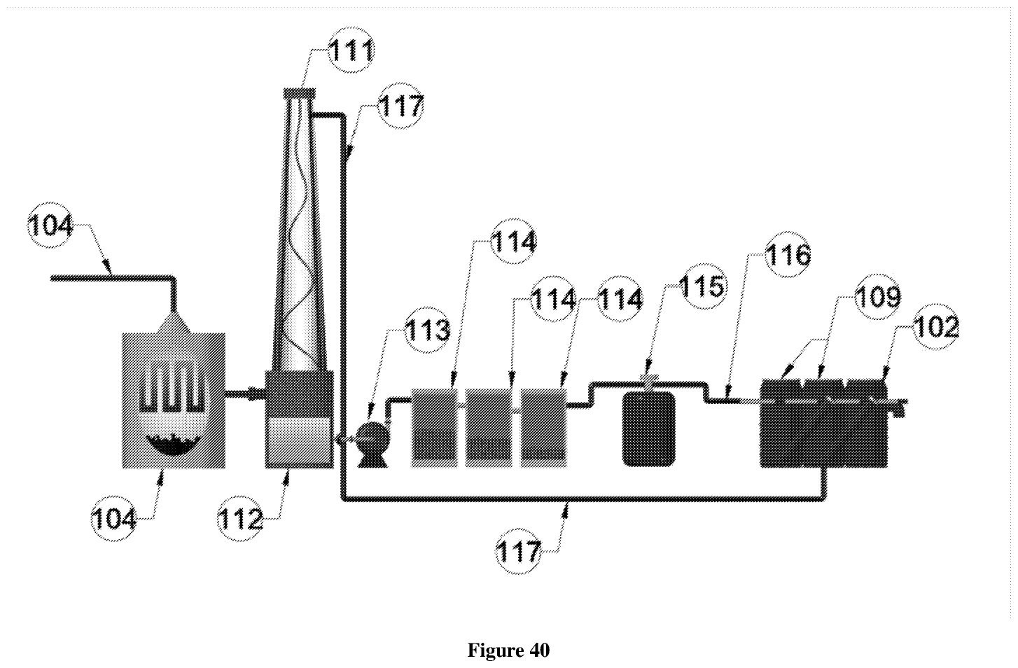

FIG. 33 shows 3 hexagonal shaped housings showing how shapes can fit together to optimise space usage. FIG. 34 is a side view of the configuration in FIG. 33 showing the as the water to be treated passes into chamber 88, the water now passes the 253-nanometer tubes of varying outputs of energy at the base of chamber 88 ozone bubbles are injected and rise upward through the liquid. The liquid is then pumped to the top of chamber 89 and the process repeats. The water enters at the top of chamber 87 and passes down through the treatment chamber, exposed to ozone producing UV light tubes of 180 nanometers. Rising from the bottom of chamber 87 is ozone enriched air injected through a diffuser assembly. After reaching the bottom of chamber 87, water will pass through the angled connection conduit into chamber 88. The UV lights in chamber 88 will be 253 nanometers or similar. Water will pass to chamber 89 where it will meet with large numbers of varying nanometers of light waves from the very lowest from the very highest all mixed together. Extreme advances oxidation preferably occurs in chamber 89. Excess gases will be vented from the top and will be carried away for gas destruction.

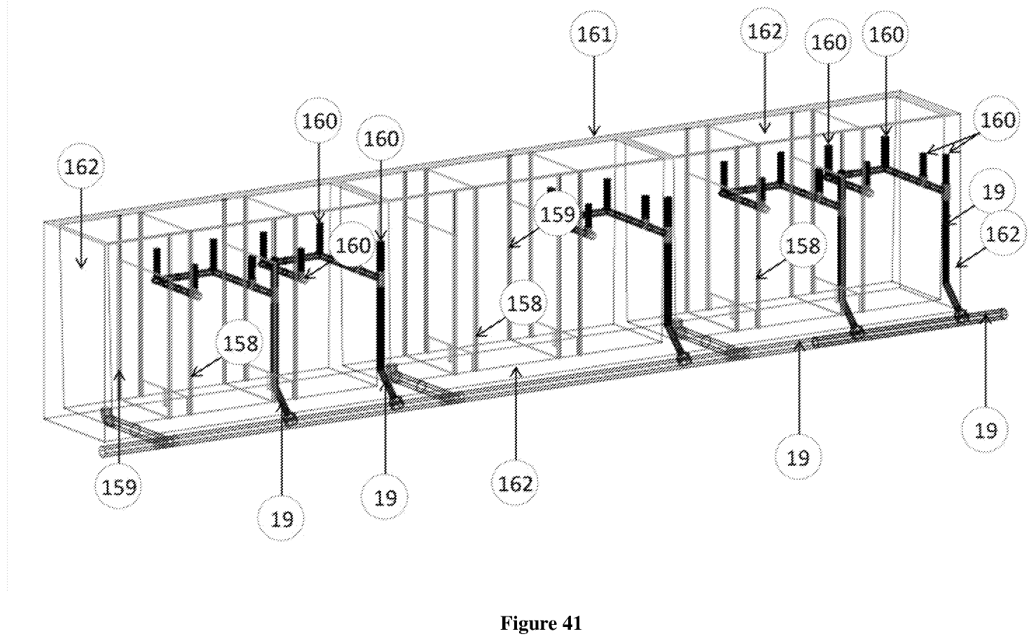

FIG. 35 shows a two 10 ft containers, one containing a sand filter or similar at the input end and another 10 ft container at the opposite and holding a discharge pump for pumping water to the town supplies on either side of a central 20 ft container showing a 4-tank system of reaction chambers connected by angled transfer pipes 90. All three of the containers sit on top, of a partly buried 40 ft. container 208 which is used for clean water storage. This water is delivered from the four reaction chambers by discharge pipe no 203. Also shown, is the air compressor 165 which is used for supplying air for the making of ozone. Discharge pump no. 201 is mounted above the clean water storage tank using suction pipe 202.

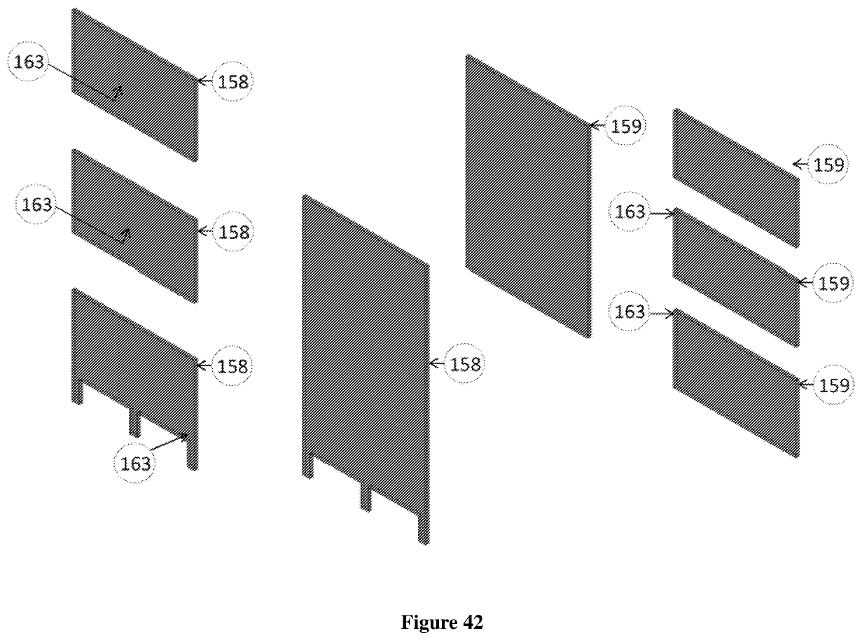

FIG. 36 is an overhead view of the configuration in FIG. 35 showing the deep well pump no 201 at the inlet side which delivers the untreated water to the 10 ft. container which has been converted to a sand filter which pre-filters the water before delivering to the four reaction chambers. Also shown are the air pump 165 for ozone production and the clean water delivery pump 201 on the outlet side.

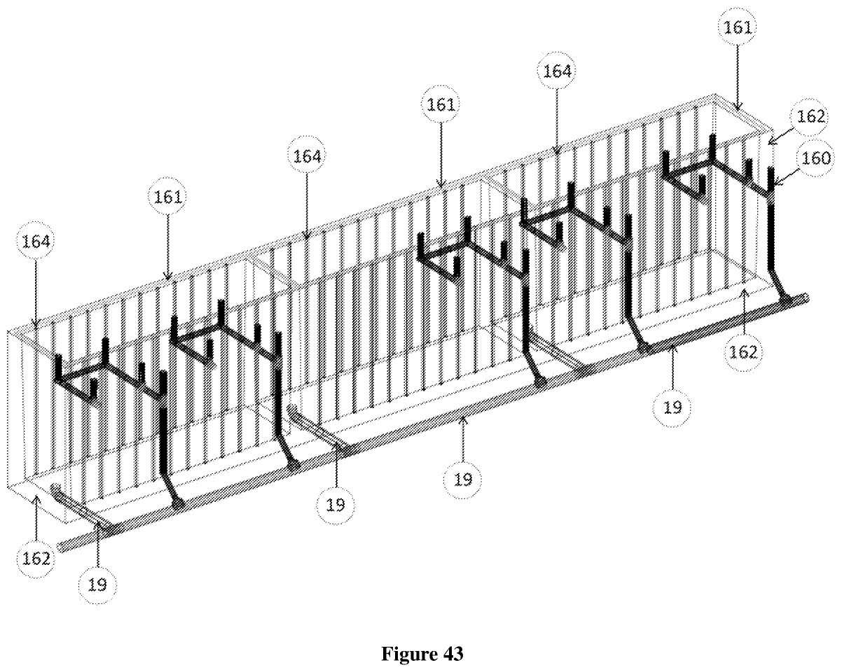

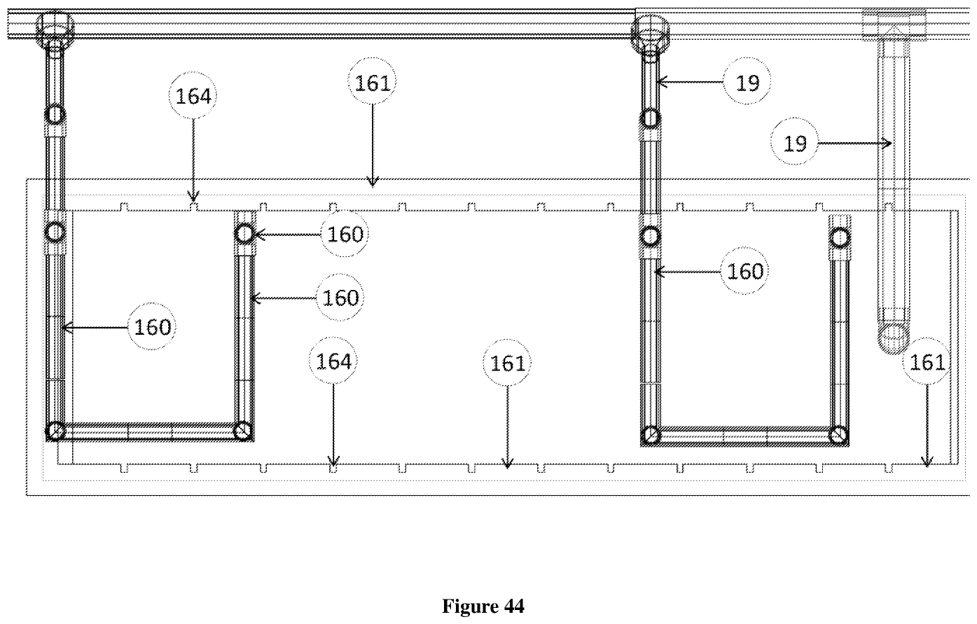

FIG. 37 shows the chemical structure of dioxin (without end Chlorines). FIG. 38 shows the molecular structure of dioxin post treatment in a system of a preferred embodiment in a lower oxidation state due to the ozone treatment.

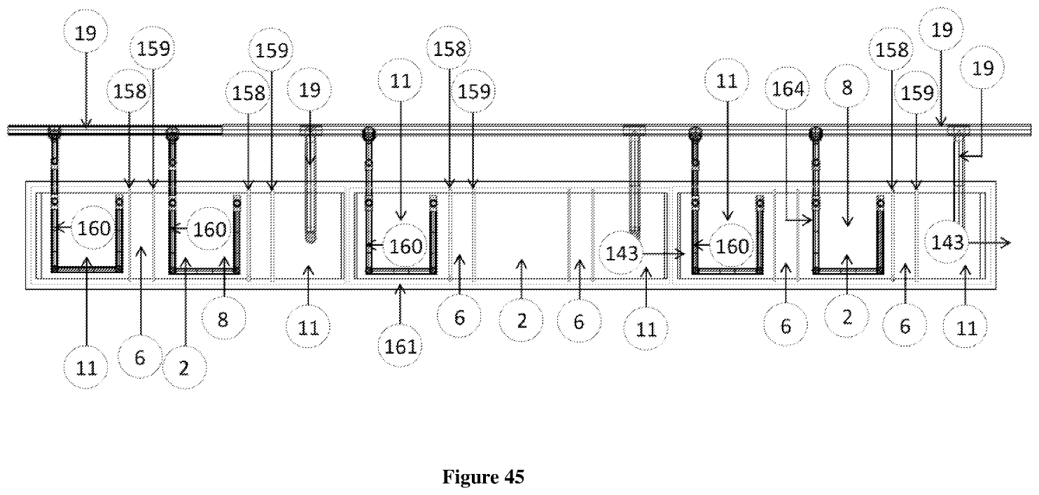

FIG. 39 illustrates a system for reducing the salinity of seawater for use in boilers or any other system as required, utilising at least two treatment systems of the present invention, a first 109 for treatment of input water in a desalination system to create chlorine gas fed to the boiler as fuel and a second 102 treating burned gases leaving the boiler to be destroyed. Clean air 103 is showing leaving the gas destruction system. The steam 104 is showing entering the steam turbine to generate electricity, which passes to the power grid 107, and additional energy is the return to operate the water desalination system and gas destruction system free of charge.

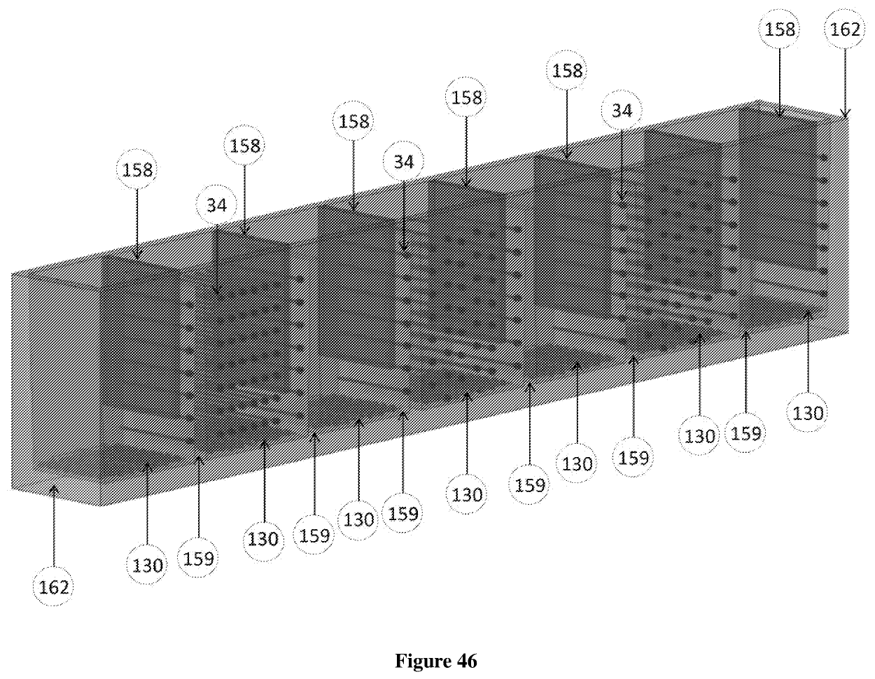

FIG. 40 shows a coal fired power station which emits a large amount of pollution into the atmosphere shown to produce clean energy because of the ability of the treatment system 109 to take the waste output absorbing into water 112, along with filtering of the ash 114 and CO₂ to then remove the sludge from the water, take the water to the treatment system 109 for destruction of heavy metals and chemicals and recirculate semi-clean water 117 back to the stack for continued reuse also showing gas destruction.

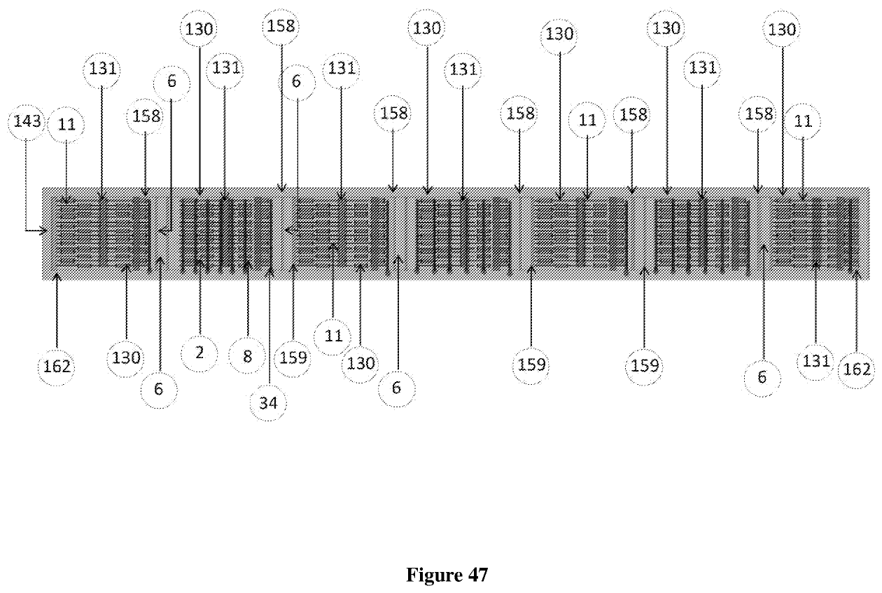

FIG. 41 shows the main external housing used to create an under and over water treatment system of a preferred embodiment. Also shown are the waste adjustable fractionation pipes 16, 0 waste drain 19, main body 162. The location of the flow control panels to create the under and over flow path are shown at 158 and 159.

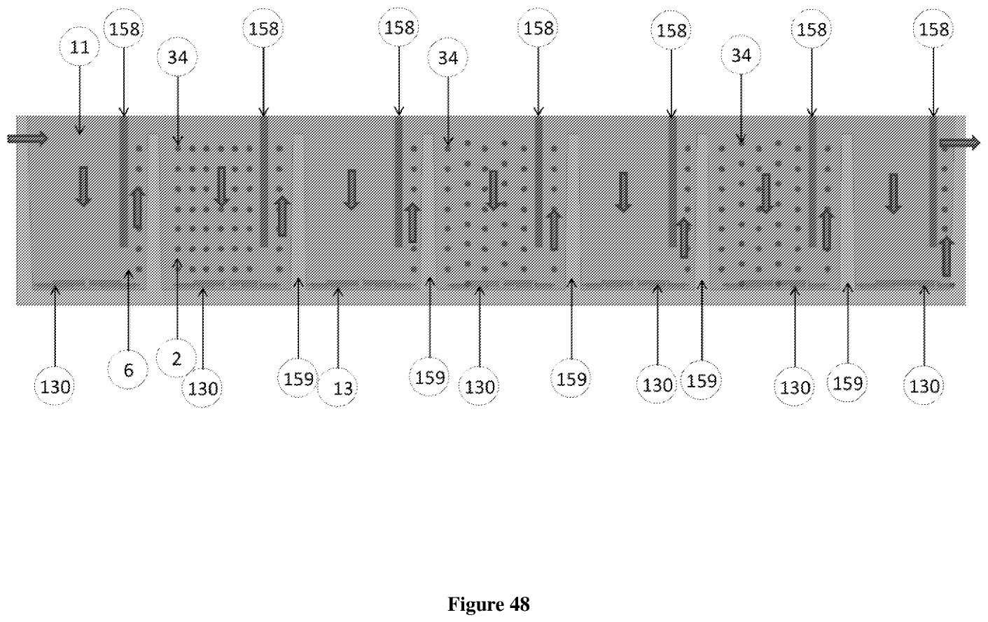

FIG. 42 shows various configurations of possible flow control panels in single sections and in multiple sections. The multiple sections are designed to be placed in the system illustrated in FIG. 41 for easy installing and removal and for any flow path and/or fluid height adjustment.

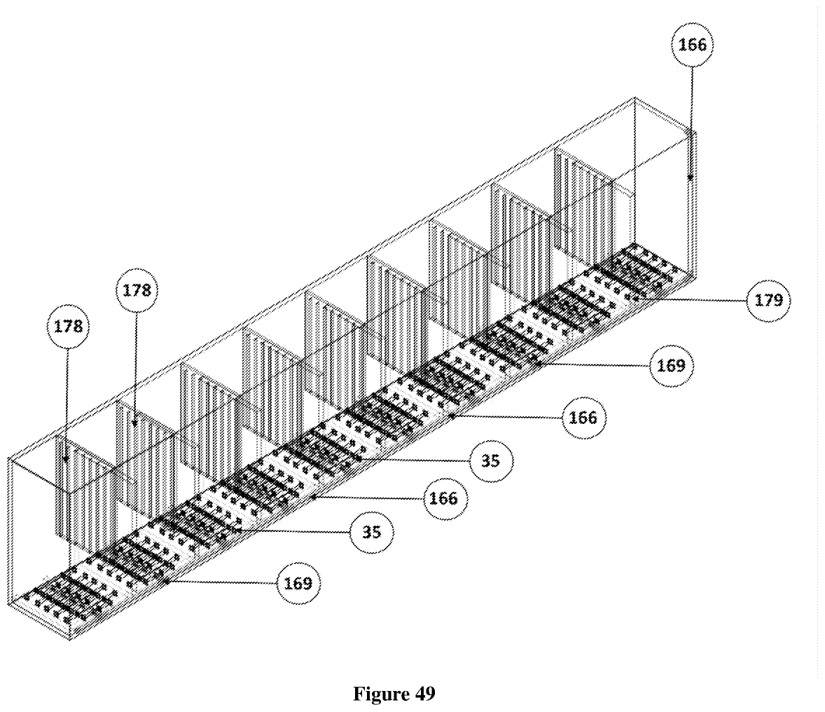

FIG. 43 shows the main body 162 of this embodiment with grooves or slide sections built into the main body to allow for the under and overflow control panels at 158 and 159 to create as many adjustable reaction chambers as are required. The advantage of being able to change the volume of the area between the sections is to assist in the application of advanced oxidation, extreme advanced oxidation, and ozone saturation for oxidation.

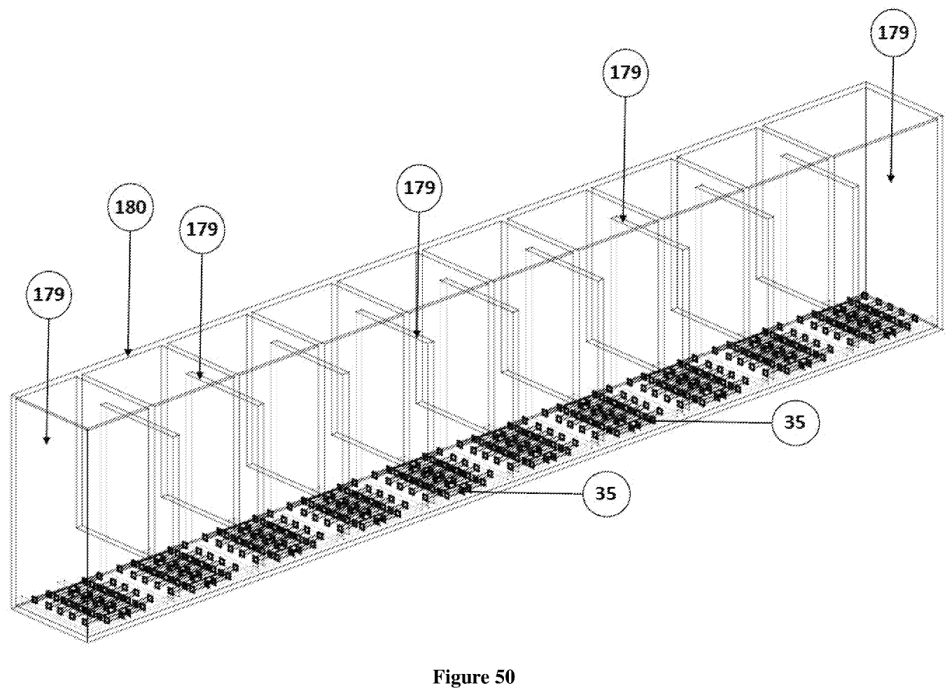

FIG. 44 shows a top view of a part of the main body of FIG. 41 showing the slide guide notches allowing placement of the panels as desired from above as well as the adjustable fractionation pipes 160 and the main body of the treatment chamber housing 161.

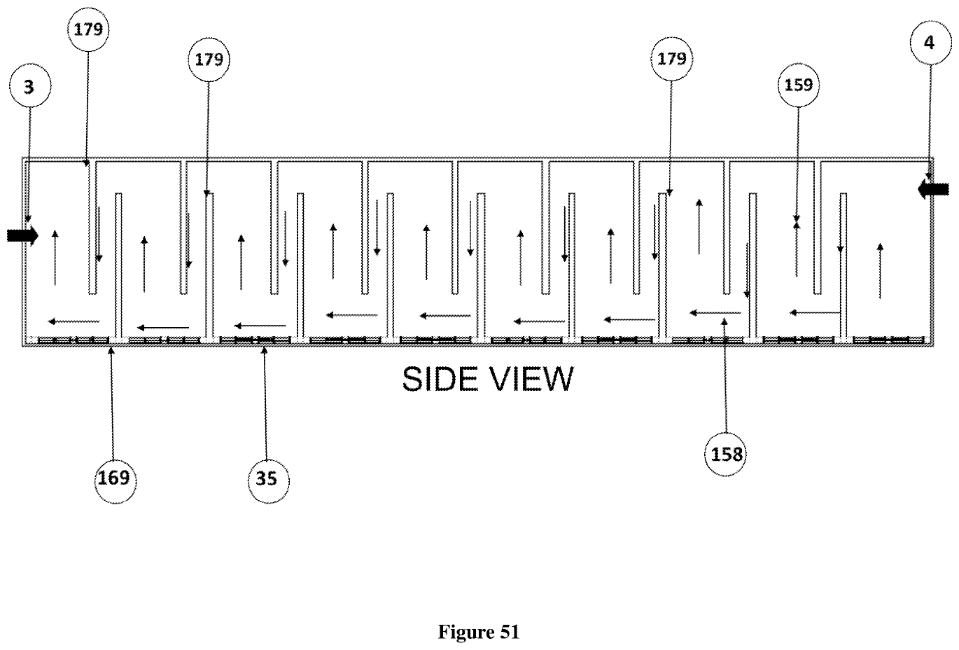

FIG. 45 shows an overhead view of an embodiment which has three units of FIG. 43 joined together end to end. Focusing on the right side section, the fluid or water enters the main body 143 into an ozonation area 11, then an advanced oxidation chamber 6, an extreme advanced oxidation chamber 8 with oxidation 2 occurring. As the water continues, it comes up through advanced oxidation 6 once again then finishing ozonation 11. The reactions can be changed from chamber to chamber depending on the water treatment required.

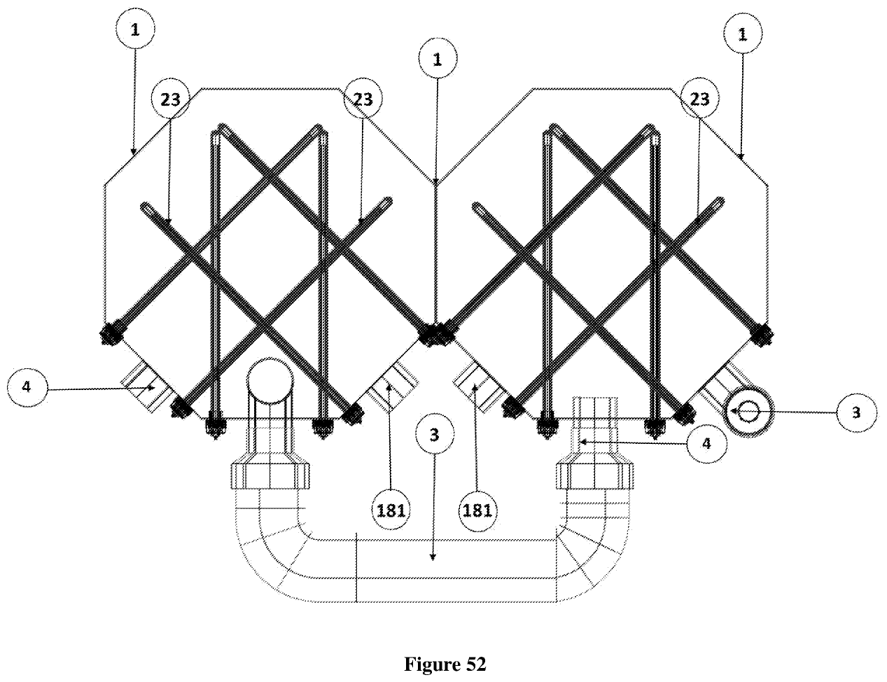

FIG. 46 illustrates the configuration shown in FIG. 43 with piping removed and dividers and UV tubes in place with the flow control panels 158 and 159, the horizontal ultraviolet lights in quartz tube 34, and air stones 130 to distribute ozone bubbles.

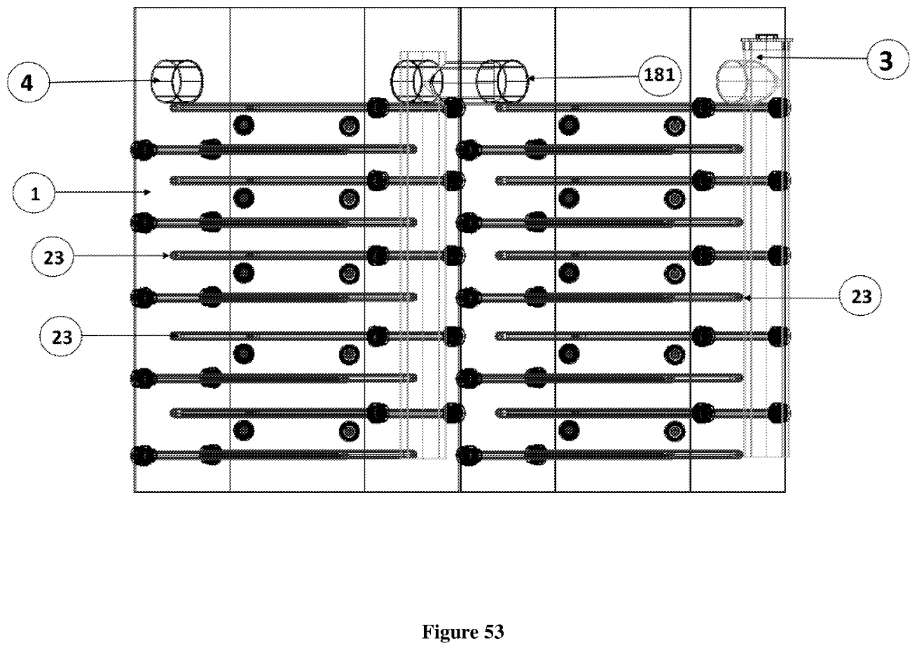

FIG. 47 is an overhead view of the FIG. 46 housing 162 with air stones 130 delivering ozone bubbles and flowing through advanced oxidation chamber 6 into extreme advanced oxidation chamber 8 and oxidation 2 then flowing up and over panel 158 and 159. This flow pattern is continuous from one end of embodiment out the other end. This configuration can be changed and varied simply by turning off ultraviolet lights 34 in some of the chambers and the flow system can be adjusted by moving the flow control panels 158 and 159.

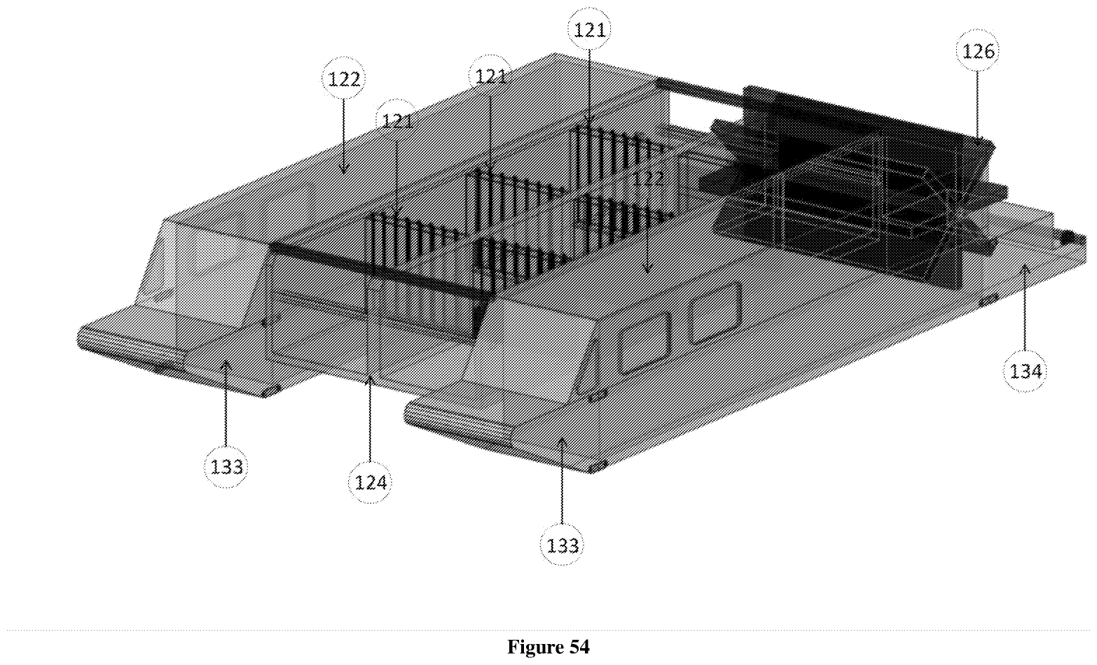

FIG. 48 shows the side profile of this embodiment with the ultraviolet lights 34 and advanced oxidation section 11. The water then enters the advance oxidation section 8 where oxidation 2 is occurring and flows continuously down against the rising ozone bubbles from the diffusion assembly 130. The water swirls through this chamber, and continues up and over flow control panels 158 and 159 into the ozone saturation chamber 11. The horizontal ultraviolet lights can set in any practical pattern, which serves to create extreme advanced oxidation conditions.

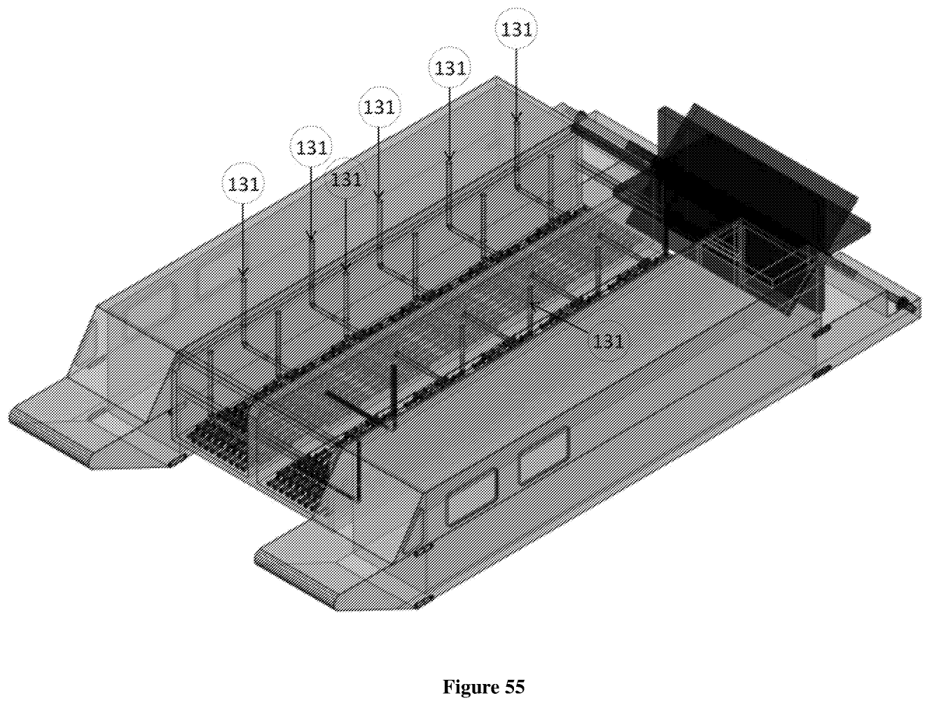

FIG. 49 shows a rectangular housing allowing under and over control flow panels with flexible disruption baffles 178 to maximise turbidity in the treatment chambers. This configuration also shows the air stone diffusion assemblies in the base of the treatment chambers.

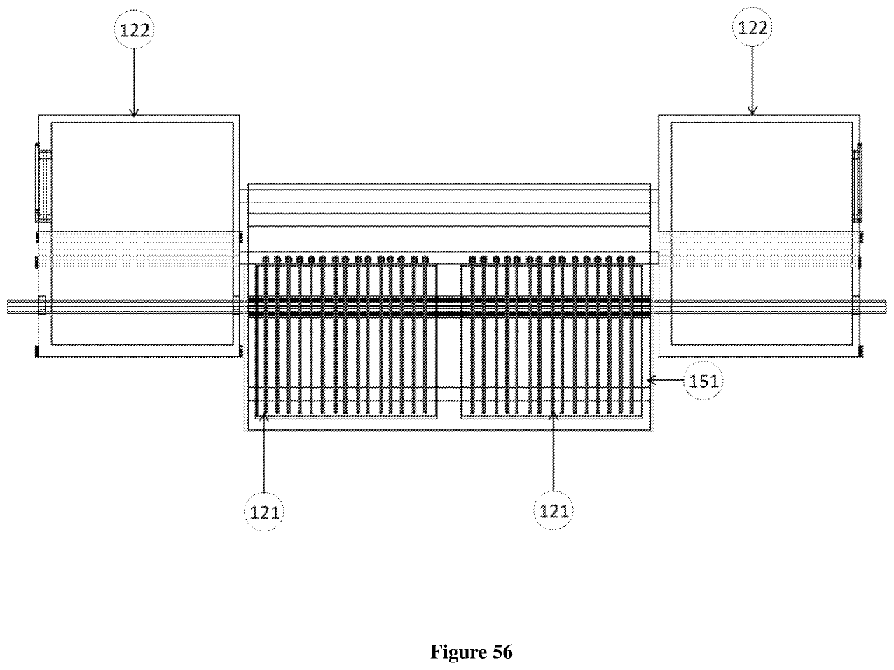

FIG. 50 is an isometric view of a rectangular housing with fixed, planar dividers which are offset side to side as well as top and bottom to form an under and over follow pattern but also a side to side lateral flow pattern through successive treatment chambers. This configuration also shows the air stone diffusion assemblies in the base of the treatment chambers.

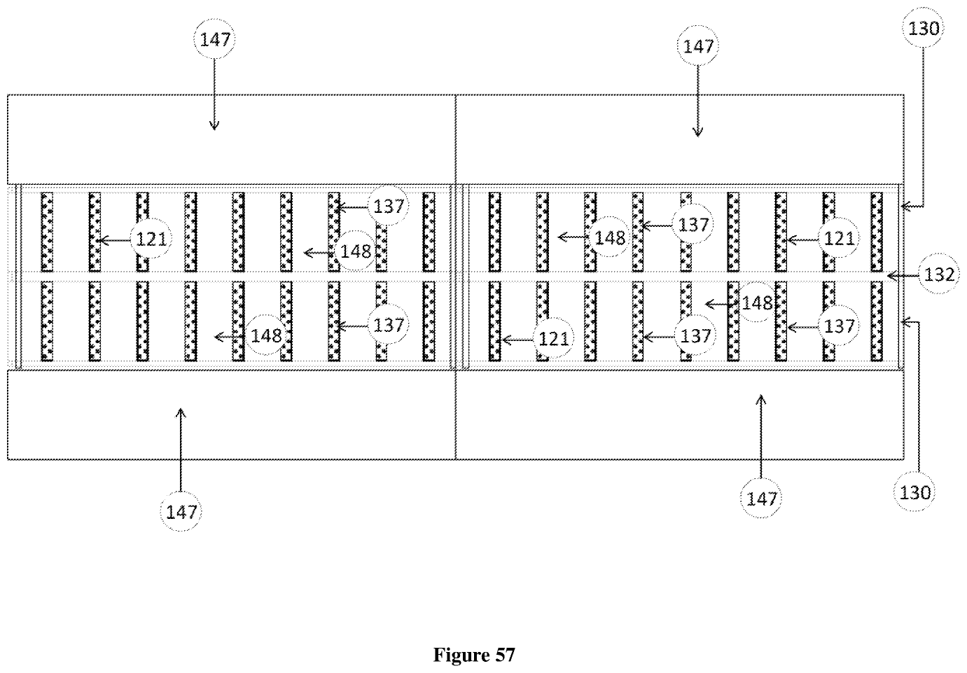

FIG. 51 shows a side view of the embodiment in FIG. 50. Not illustrated in this Figure are the UV bulbs, which have been removed for clarity.

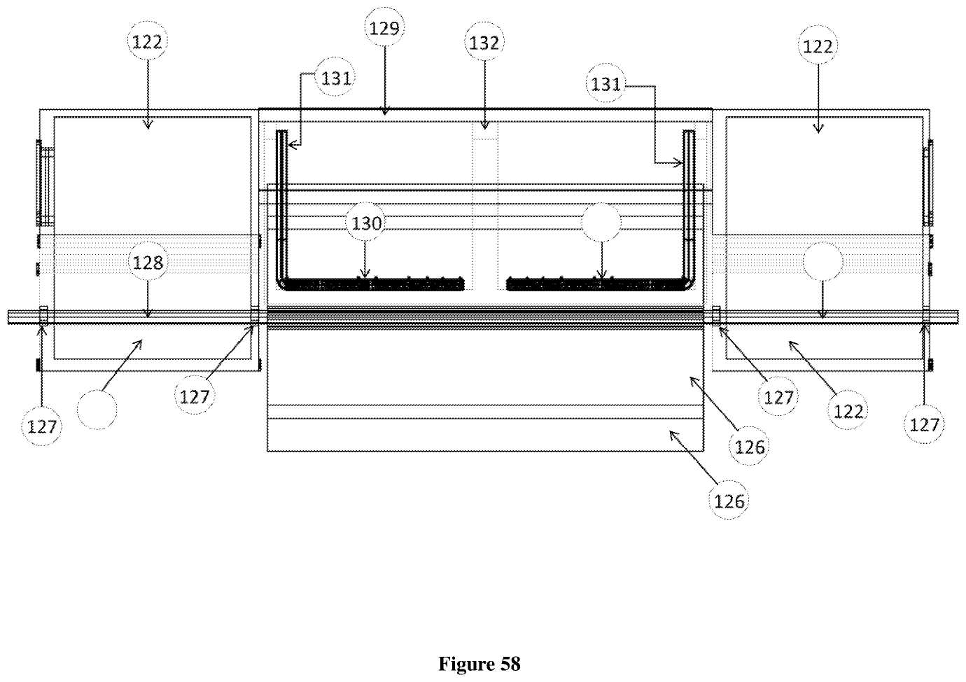

FIG. 52 shows an overhead view of two treatment chambers connected in series where water to be treated is being feed in at the top on the left-hand side, then works its way to the bottom or at least closer to the air stone diffusion assembly at the bottom of the treatment chamber, then rises up from the bottom of the base through an internal pipe which is connected horizontally or at an angle to the second treatment chamber, once again entering at the top and working its way against the flow of the ozone bubbles, working its way through the UV light waves to the base of the treatment chamber then rising to the top through an internal or external pipe to be discharged into clean water storage. Components 181 are additional outlets which can be used to drain any excess frothing or bubbling compounds from the surface of the water to waste.

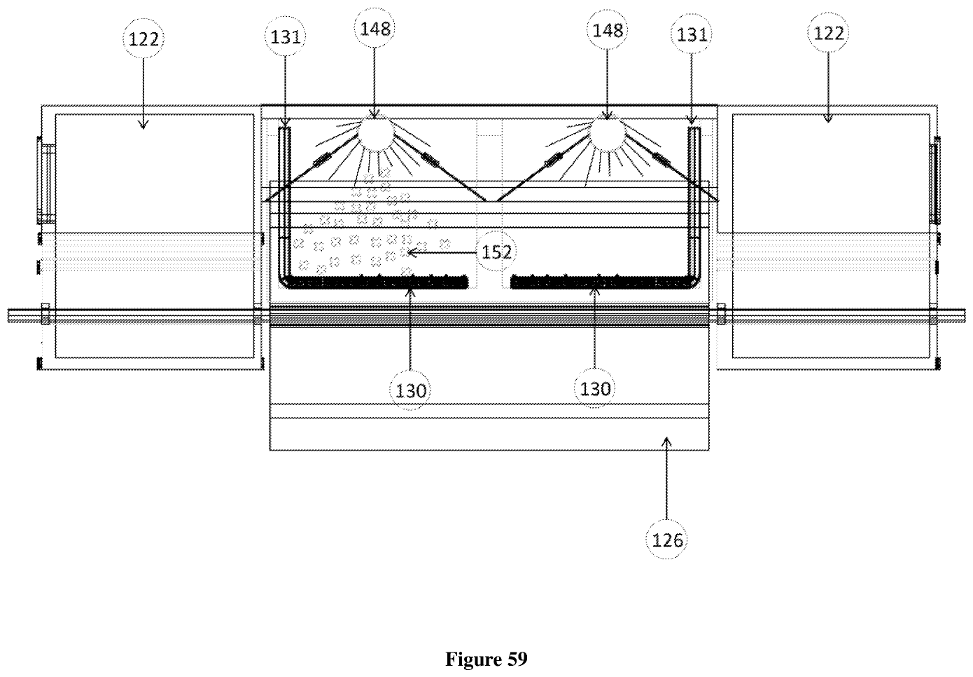

FIG. 53 is a side view of the configuration illustrated in FIG. 52 showing internal components and connection conduit starting near the base of the treatment chamber and carrying transfer water to the joining chamber. In this configuration, this is an internal pipe not an external pipe.

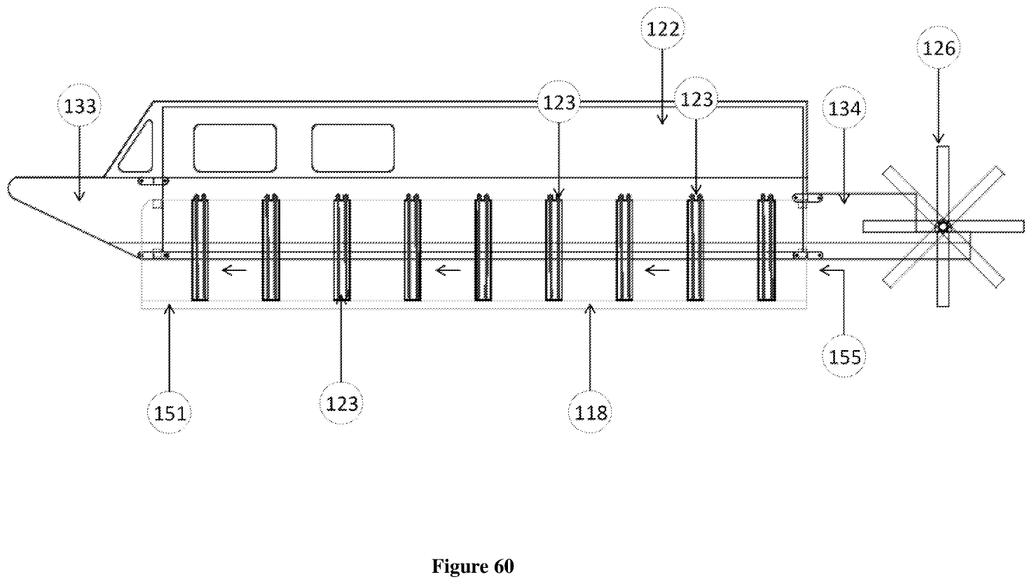

FIG. 54 is an isometric view of a waterborne vessel configuration according to an embodiment. This embodiment is formed using two shipping containers acting as two boat hulls in a catamaran or multi-hull 134 and a rear paddle wheel 126 which is used to drive the vessel through the water and to drive water over an entry end of the hull configuration. Sloping forward bow sections 133 have been added to this configuration. The central treatment area of this embodiment is preferably movable relative to the hull portions 134 in order to raise (transport position) and lower (treatment position) the central treatment area. In the stern section, lifting and lowering mechanisms are typically provided. This Figure highlights the UV lights in the treatment chambers, oriented vertically and spaced over the length of the treatment chamber.

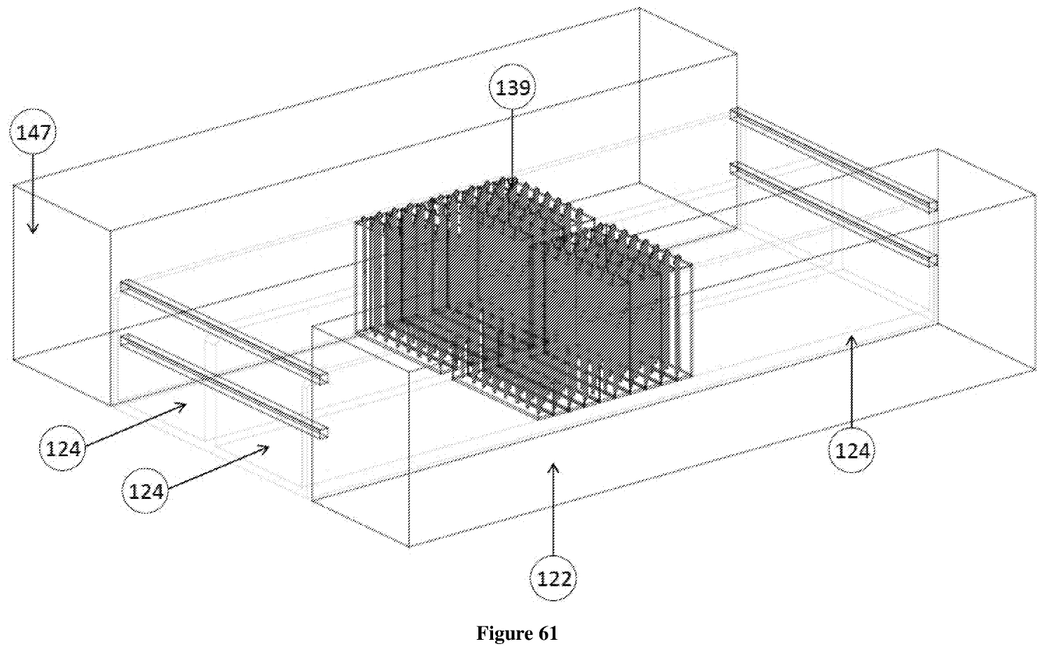

FIG. 55 shows the embodiment of FIG. 54 but highlighting the position of the air stone diffusion assemblies attached to the bottom of the platform which has been lifted clear out the water for vessel travel. This Figure also shows the ozone or air delivery pipes which carry ozone or air to the air stone or venturi diffusion assemblies which are attached to the base.

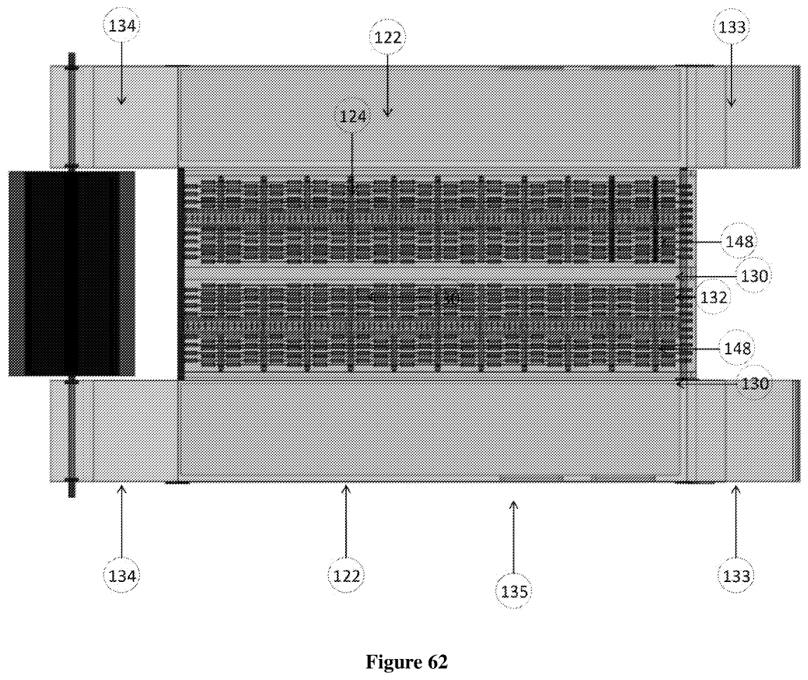

FIG. 56 is an end view of a vessel of FIGS. 54 and 55 with a central portion 151 with UV treatment lamps in a submerged operating position.

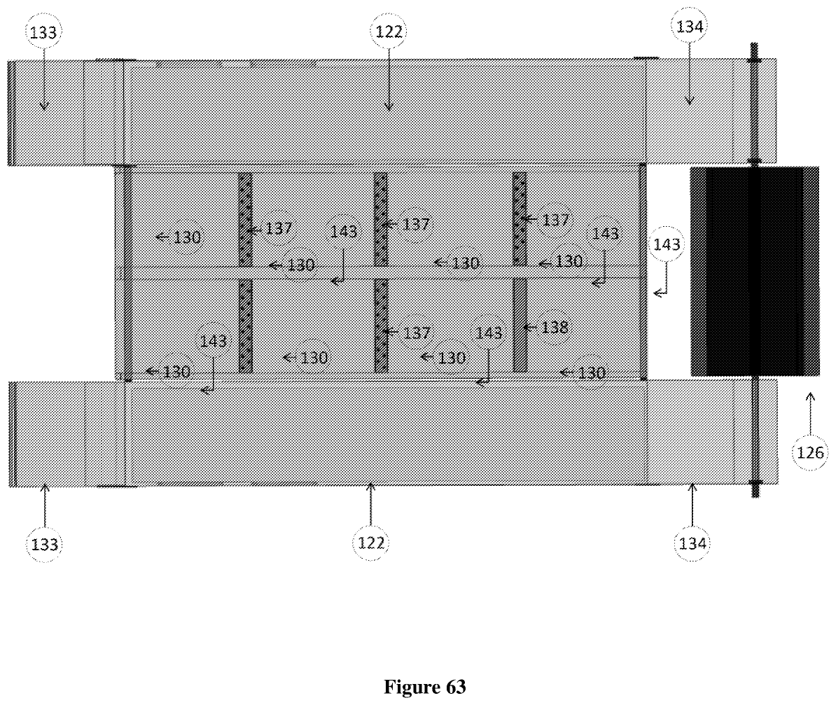

FIG. 57 shows an overhead view of four shipping containers joined together as a single catamaran or land based system (without the ends). Also shown is a series of horizontally extending, evenly spaced ultraviolet tubes sitting over the submerged air stones or venturi diffusion assemblies.

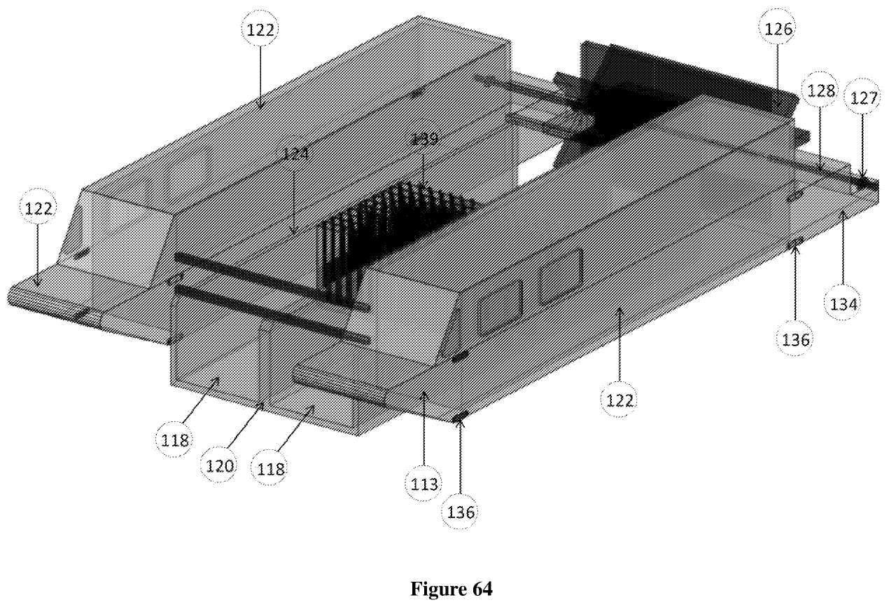

FIG. 58 is an end view of a vessel of FIGS. 54 and 55 with a central portion in the raised position and highlighting the location of the air stones and delivery pipes.

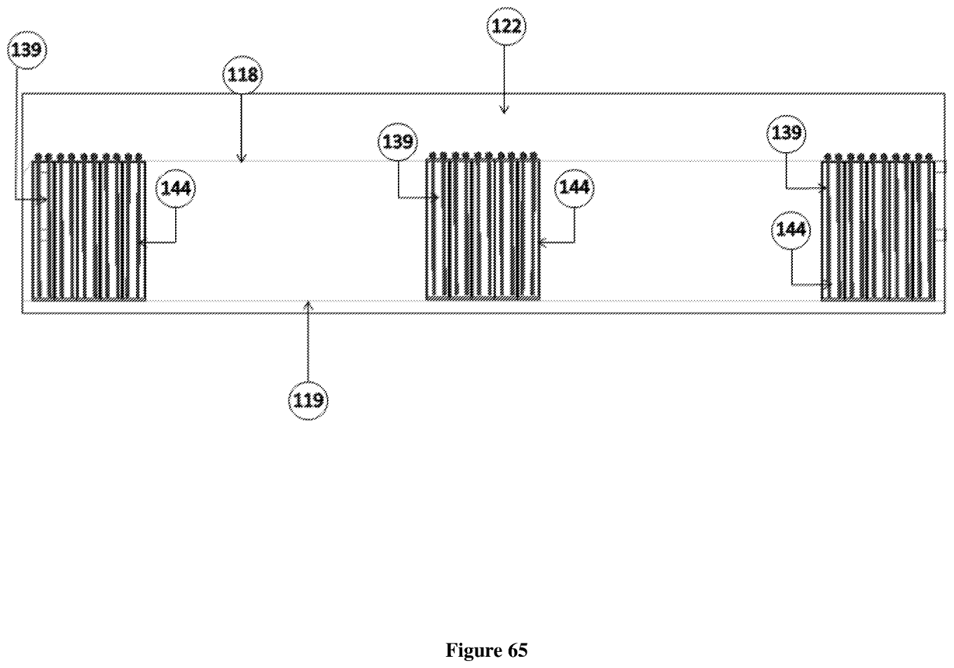

FIG. 59 shows the treatment chambers in a lifted position with water pipes raised above showing biological media 152 in position the water delivery pipes 148 are designed to spray polluted water over the media 152 as a biological treatment or for removal of ammonia and other toxins for example, for treatment.

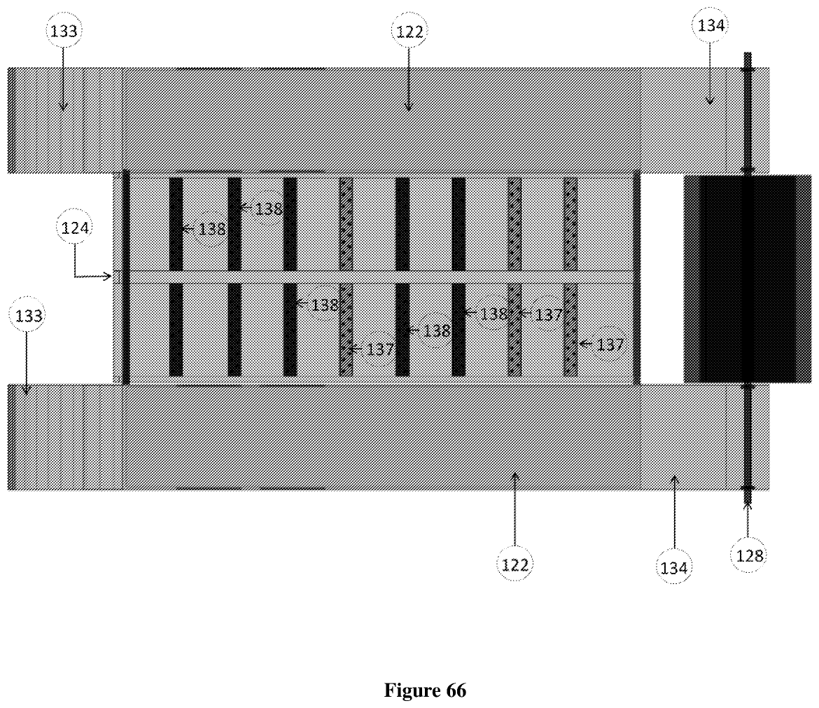

FIG. 60 shows the side profile of the water vessel embodiment with a bow 133 and stern section 134 bolted to main container body 122. Each section is individually sealed before attachment to the main body 122. Also showing is the position of the lifting platform in the lowered position and the height of the paddle wheel. The movement of the vessel by the paddle wheel pushes water to be treated through the treatment chamber and through the ultraviolet bulbs and pass the ozone bubbling air stones and out of the end of the vessel. The speed of this flow can be controlled by the speed of the vessel relative to the water.

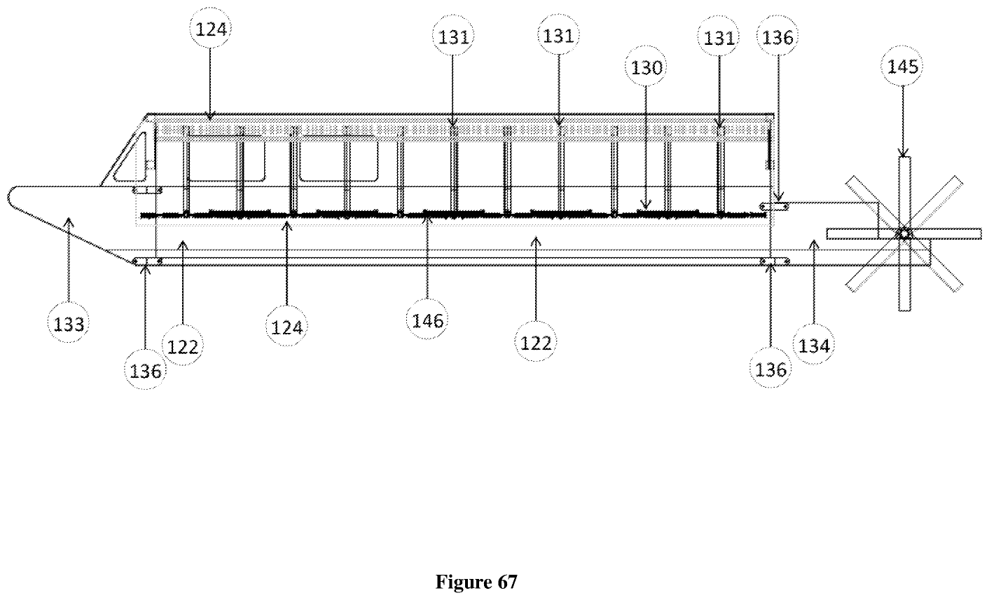

FIG. 61 shows the ultraviolet bulbs in racks 139 compressed together in a land based system which has closed sections on a lifting platform to make it similar two tanks to change reactions.

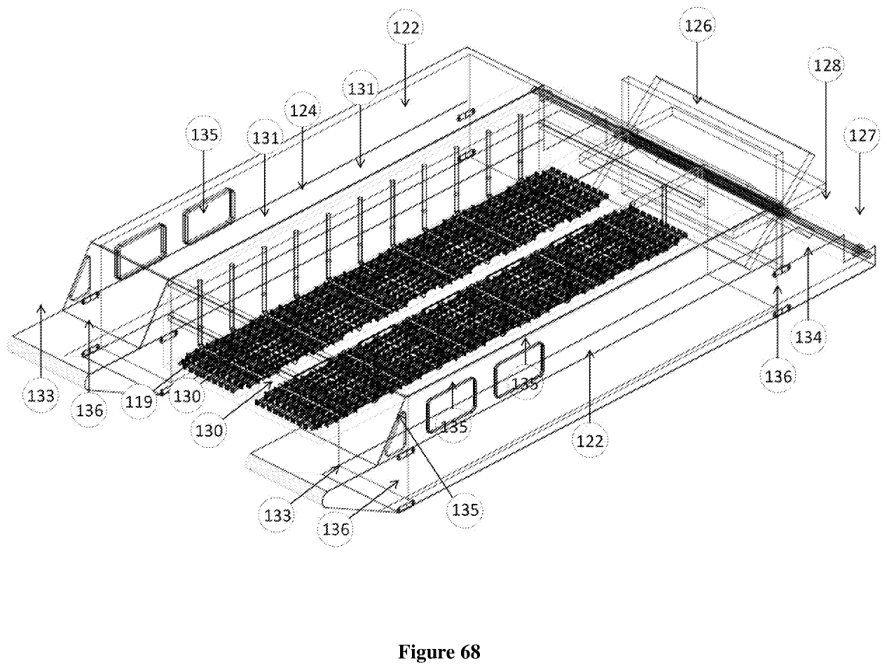

FIG. 62 shows an overhead view of the water vessel embodiment with a pair of height adjustable, side by side, longitudinal treatment chambers joined together 132 with air stones or venturi diffusion assemblies and the spray pipes 148.

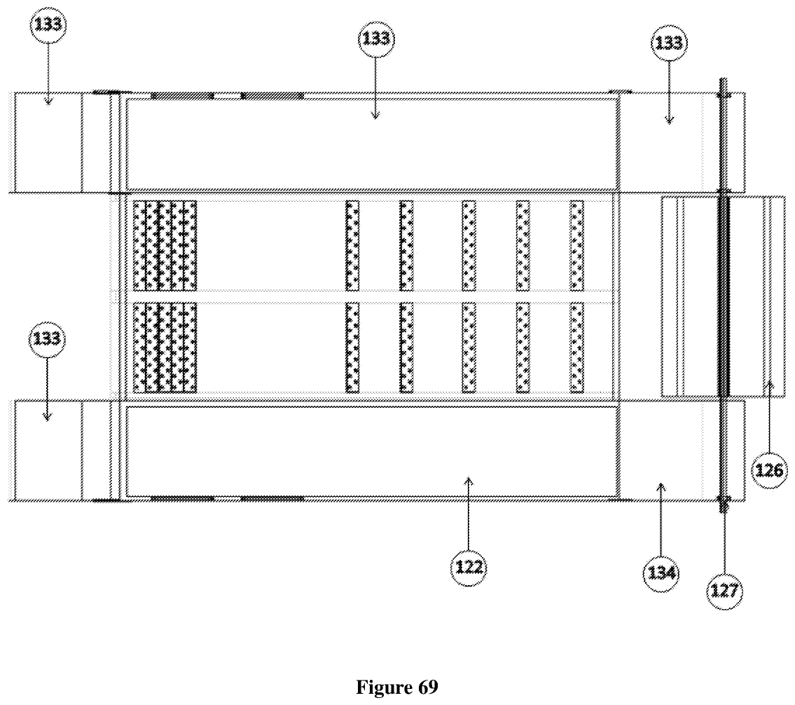

FIG. 63 shows an overhead view of water 143 being pushed from the stern to bow of the vessel over the treatment chambers 119 and through the ultraviolet bulbs no. 137 which are turned on and through ultraviolet bulbs 138 which are turned off to create a different reaction with the ozone rising. The water is moved by the paddle 126.

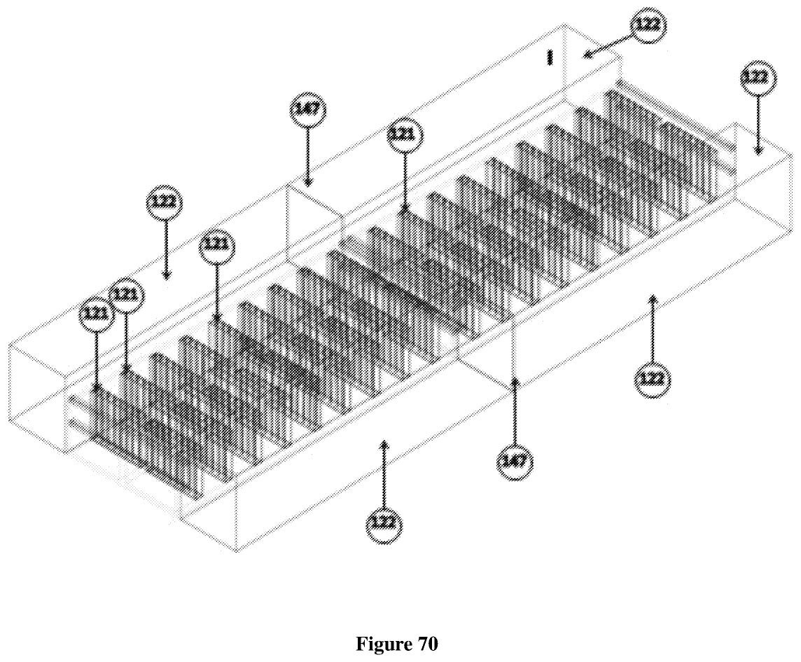

FIG. 64 shows the moveable treatment chambers in the lowered position with UV lights 139 in the compact configuration.

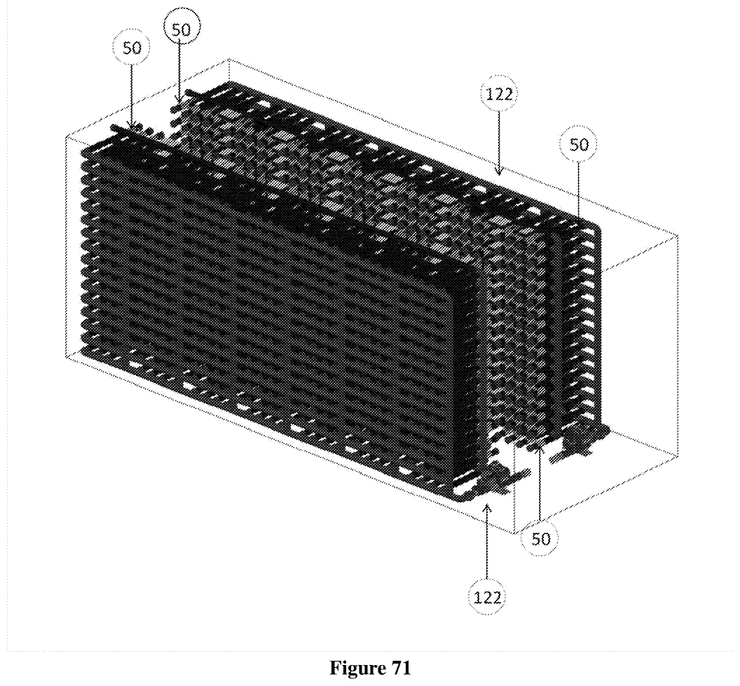

FIG. 65 shows three sets of ultraviolet light racks 139 in compact configurations to create extreme advanced oxidation when combined with the rising ozone bubbles in combination with the water being pushed or driven through the submerged treatment chamber 119.

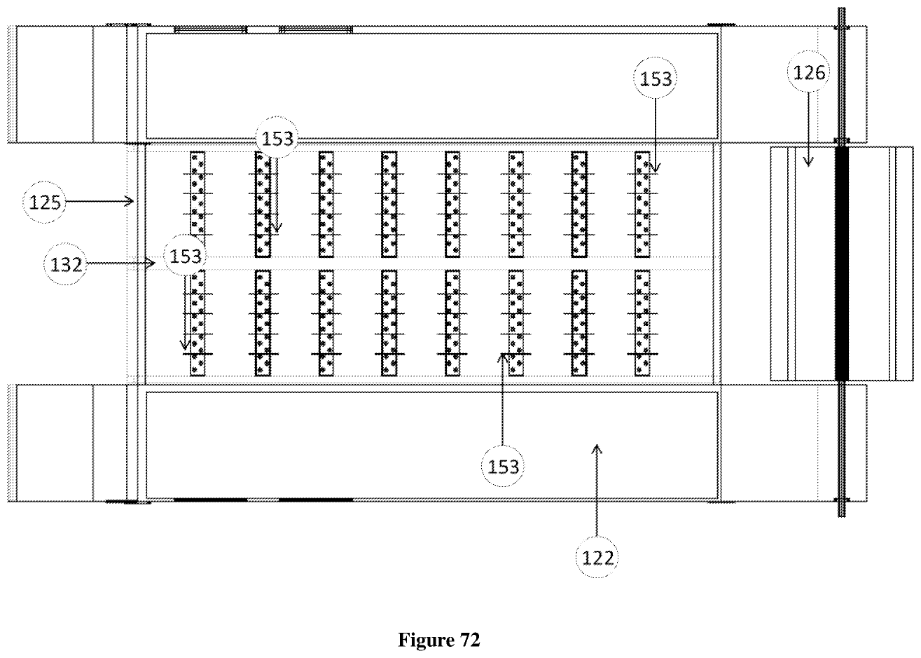

FIG. 66 shows the water vessel embodiment from above with some ultraviolet lights in the off position 138 and some ultraviolet lights turned on 137 showing that different treatment conditions can be created by turning the UV lights on and off.

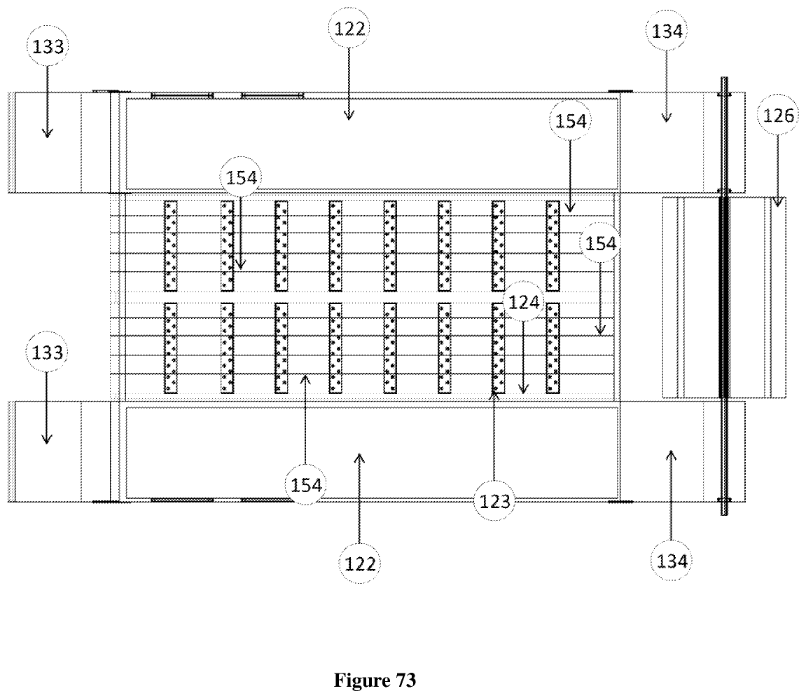

FIG. 67 shows the vessel with the water line shown with the lifting platform in the raised position 124. Also visible are the air stones or venturi diffusion assemblies 146 and the ozone delivery pipes 131. Flexible ozone pipes will normally be attached to the ozone delivery pipes to allow the raising or lowering of the treatment chambers 119.

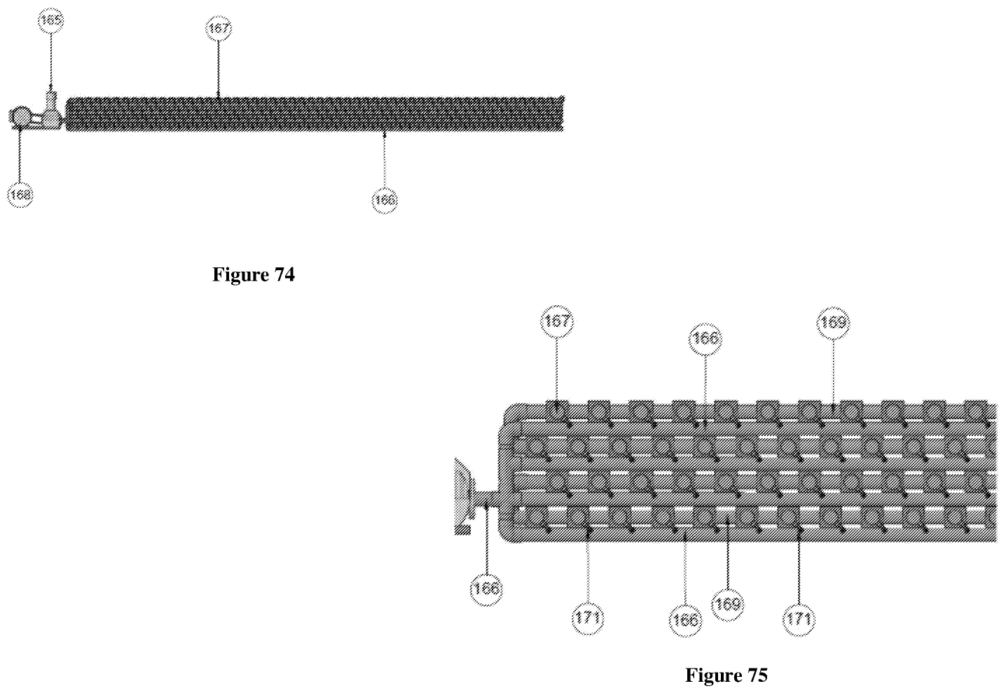

FIG. 68 shows the treatment chambers 119 including air stones and delivery pipes.

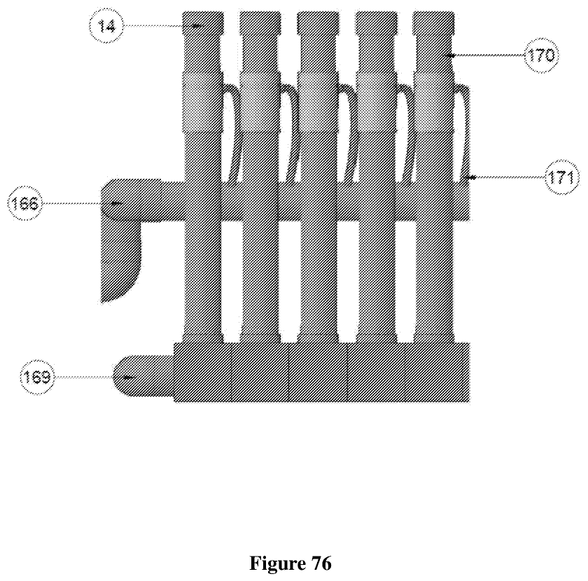

FIG. 69 is an overhead view of an alternative configuration with laterally extending UV light assemblies in racks in different positions for the production of advanced oxidation conditions, and extreme advanced oxidation conditions when combined with ozone bubbles which react as prisms or mirrors creating multiple wavelengths of lights.

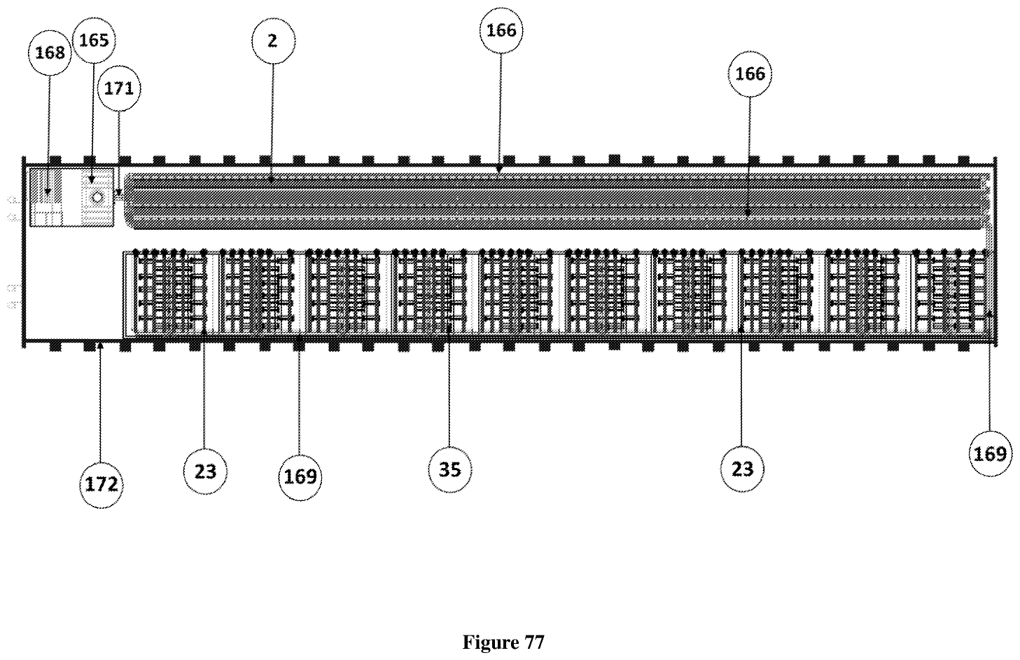

FIG. 70 is an isometric view of an alternative embodiment with vertically extending UV light assemblies past which fluid moves. This configuration shows four land containers joined with a closed end on a lifting platform to create sealed sections to hold water so that water can be pumped in one end and drained at the other end at a desired flowrate combined with the ozone bubbles and ultraviolet bulbs in racks 121 to destroy heavy metals chemicals and bacteria.

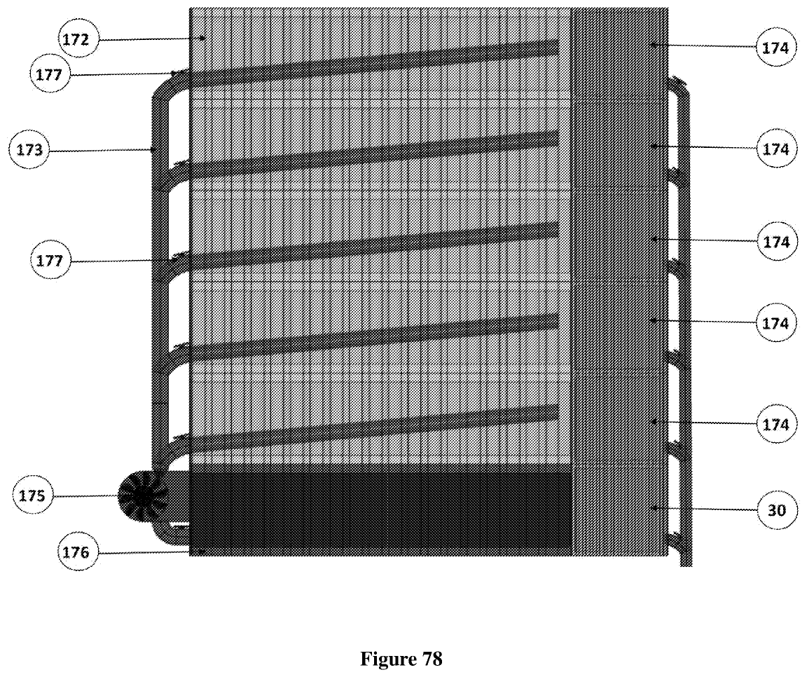

FIG. 71 shows an ultraviolet light assembly to create ozone according to a preferred embodiment. The ultraviolet lights of 180 nanometers are provided in horizontal tubes which air is then blown over to create ozone. The resultant ozone enriched air is nitric acid-free which then exits this system for use, typically in the diffusion assemblies of the present invention.

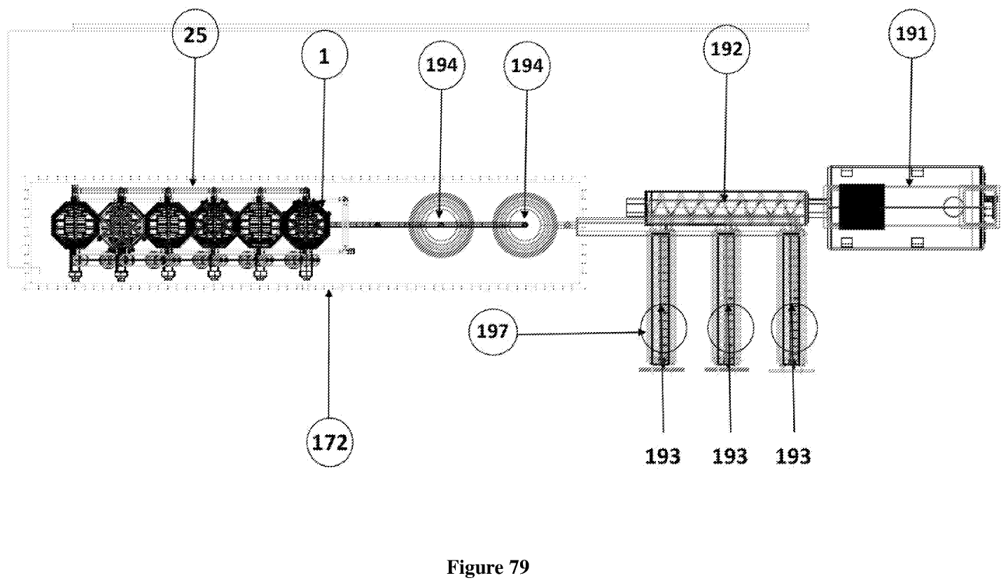

FIG. 72 shows short reflective plates spaced across the horizontally extending UV tubes to enhance the reflection of the UV light to enhance treatment using varying wavelengths of ultraviolet lights.

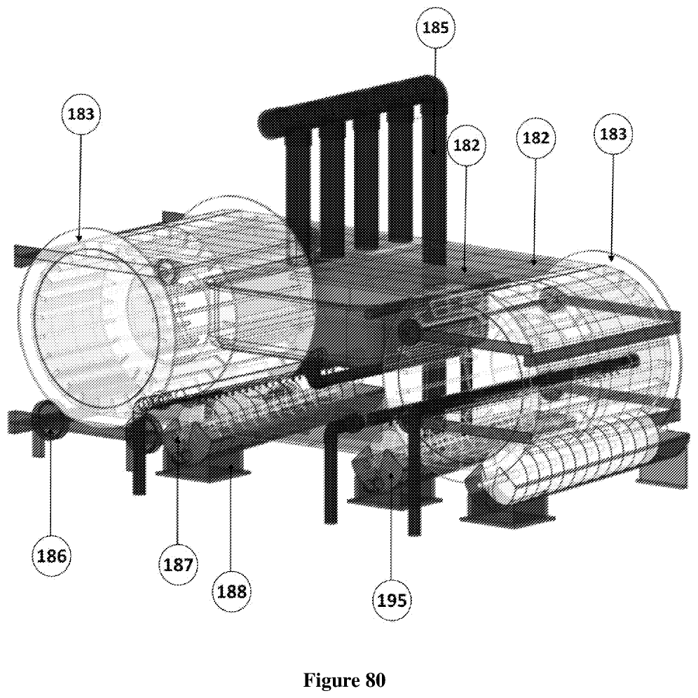

FIG. 73 shows laterally extending UV light assemblies with elongate transverse reflective plates to reflect light from light to bubbles from the plate to bubbles to enhance the treatment effect.

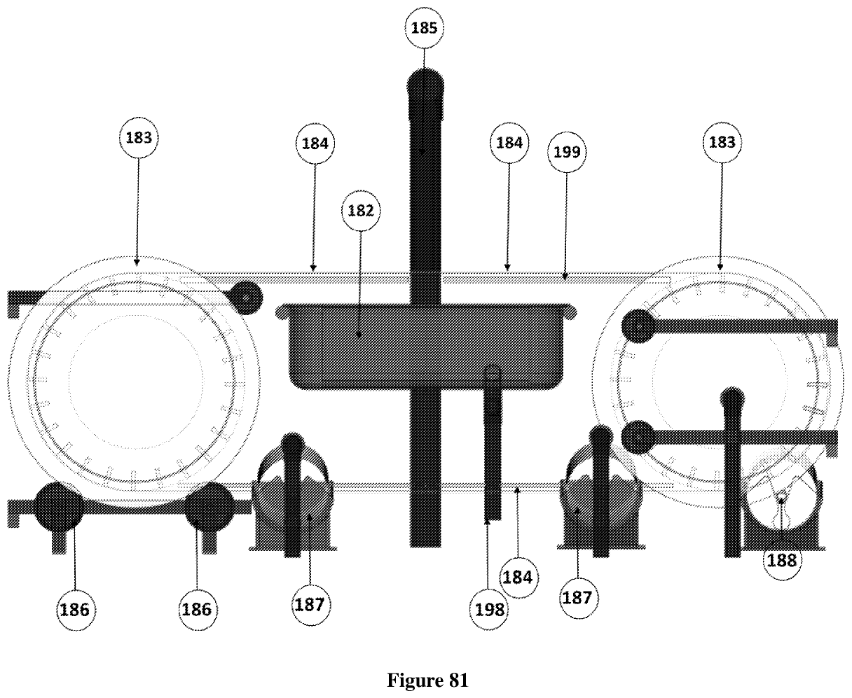

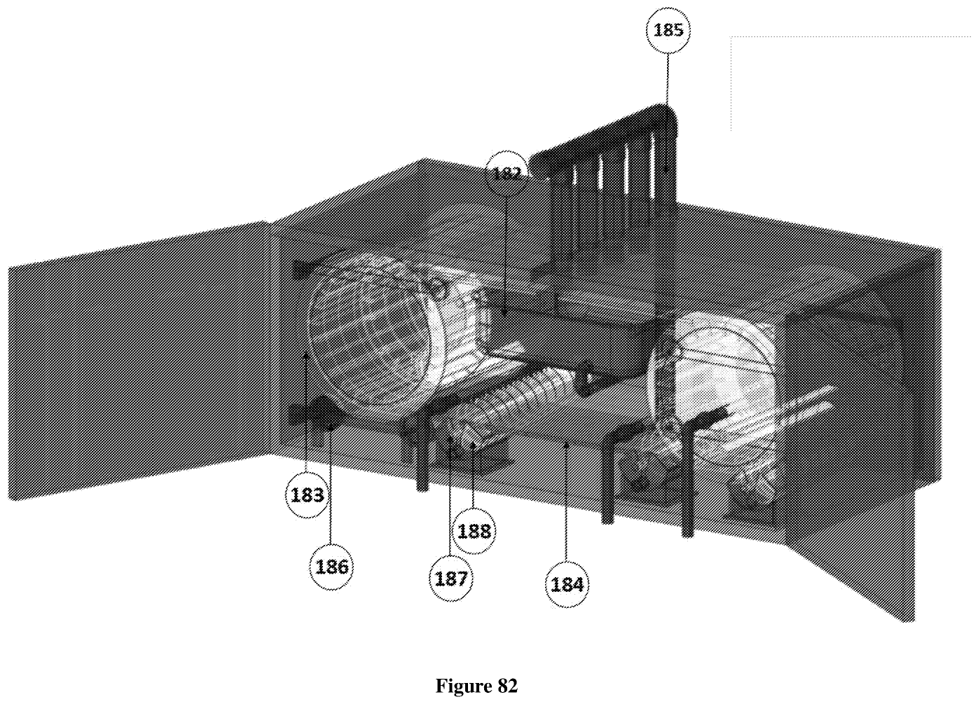

FIG. 74 shows a horizontal tube bundle to create ozone. This figure shows an oil-free air compressor or blower 165 powered by an electric motor, which is blowing or compressing air or pure oxygen into the ozone creating tubes 180 nm or similar to create ozone which will be located in close physical proximity to the treatment chambers allowing the ozone produced to be delivered within seconds to the air stones or venturi diffusion assemblies at the base of each chamber. FIG. 75 is a detailed view of the configuration illustrated in FIG. 74.

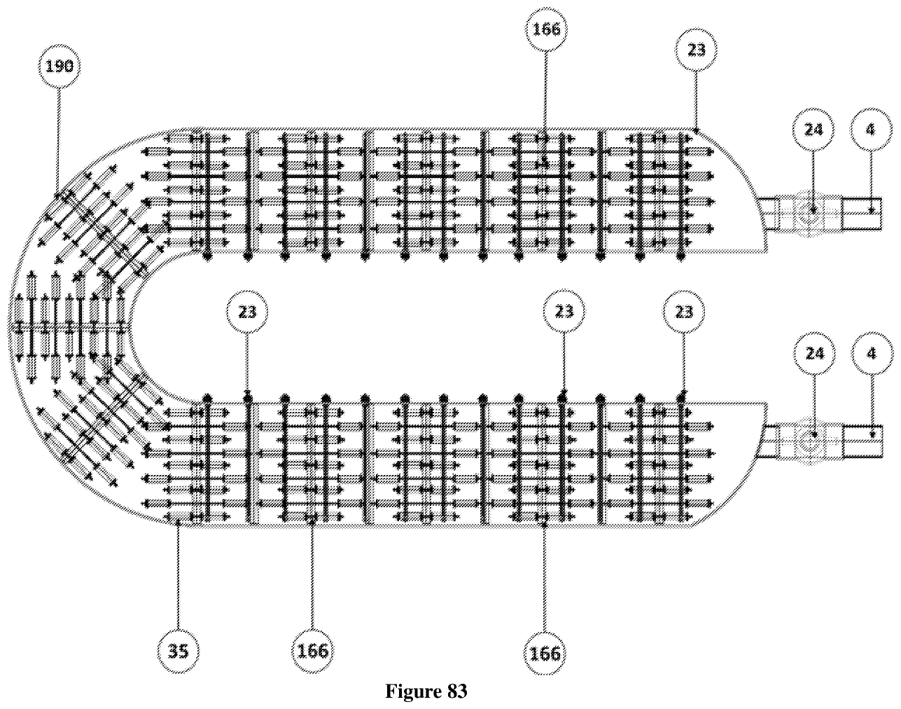

FIG. 76 is a schematic illustration of a vertical tube bundle to create ozone from compressed air or oxygen. This figure shows ozone production in ozone formation chambers in a vertical position with delivery pipes 166 delivering to a delivery conduit 171. These units can have a control valve to control the flow into ozone formation chamber, ozone being delivered to air stones or venturi diffusion assembly or to a gas outlet 169.

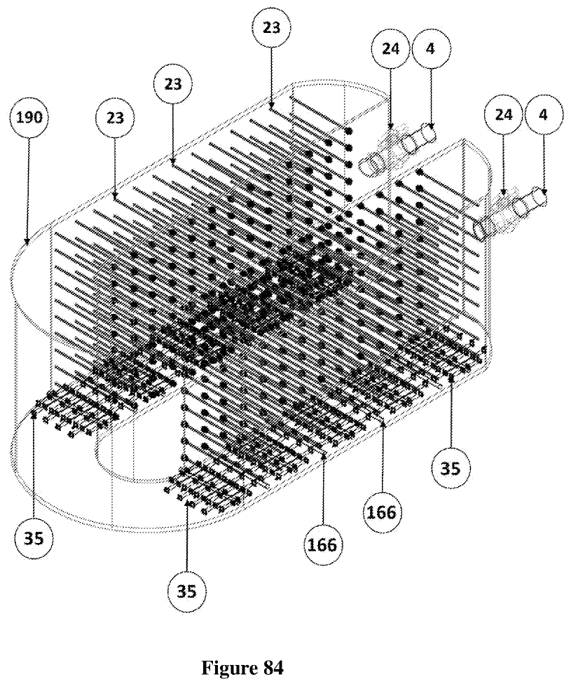

FIG. 77 shows a top view of the ozone formation configuration for FIG. 74 used to feed ozone to the liquid treatment chambers of FIG. 45 fitted in rows at the top of the shipping container or similar building. This configuration shows ozone being delivered to the diffusion assemblies at the base of the treatment chambers where it bubbles up through the fluid or water and passes the preferred horizontal ultraviolet tubes.

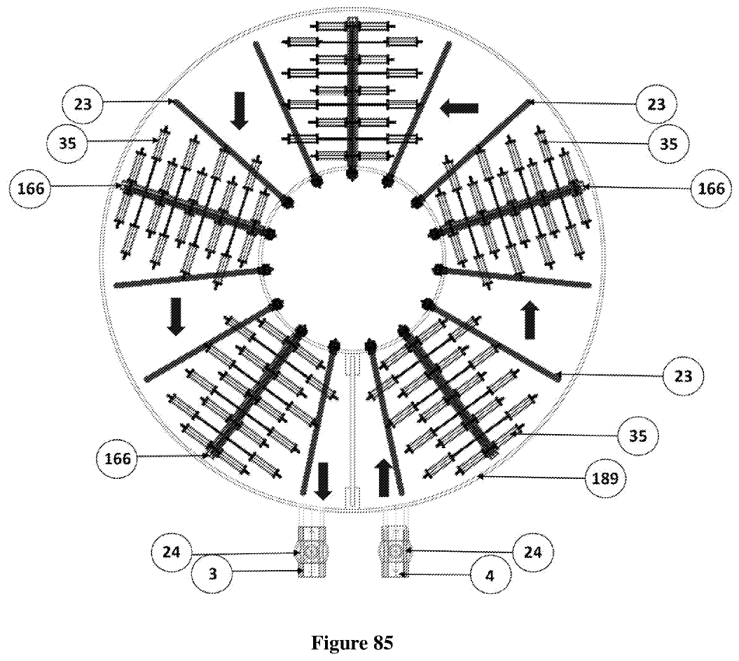

FIG. 78 is a front view, illustrates six (6) 40 ft. shipping containers or similar vessels, stacked vertically with six (6) 10 ft. shipping containers attached at one end thereof. The lowest container is a purified water-holding tank. The pipes extending from the left end side of the top five containers shows the processed clean water exiting the five containers and then falling through a turbine 175, which is spinning as a generator to create electricity, and the water then entering the water or fluid storage tank at the base. On the right-hand side, the 10 ft. containers house filtration equipment for pre-filtering of the water. The treatment chambers are housed in the top five containers. Multiples of this configuration can be stacked side by side to produce the desired output of treated water.

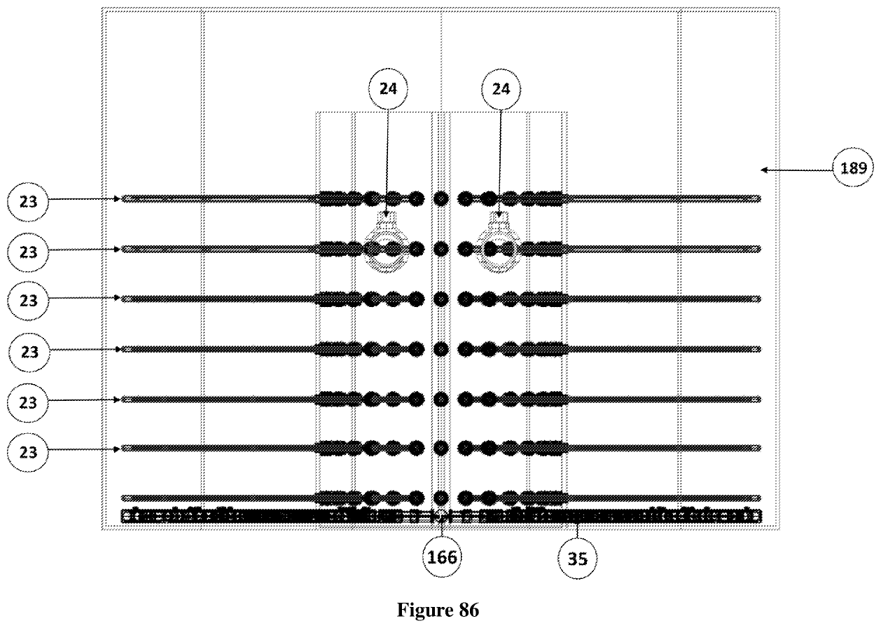

FIG. 79 is a top view of a modernized sewage treatment plant, in which solid and liquid waste enter through large angled rollers 193, fluid drops into a base tray 197. The liquids then flow into a drain no. 196 before entering the filters no. 194 pump into free flows into six polarization chambers for purification and sterilizations. The sewage solids then flow out of the long angled rotating drum filters 194 into to sealed auger 192, which is having ozone gas injected. This then flows up into a heated paddle dryer 191, when it exits the paddle dryer it is germ-free. The waste product that has been paddle heated dryer is also being ozone injected as a continuous feed comes out over a vibrating screen falling onto a conveyer belt and is conveyed for stacking and composting.

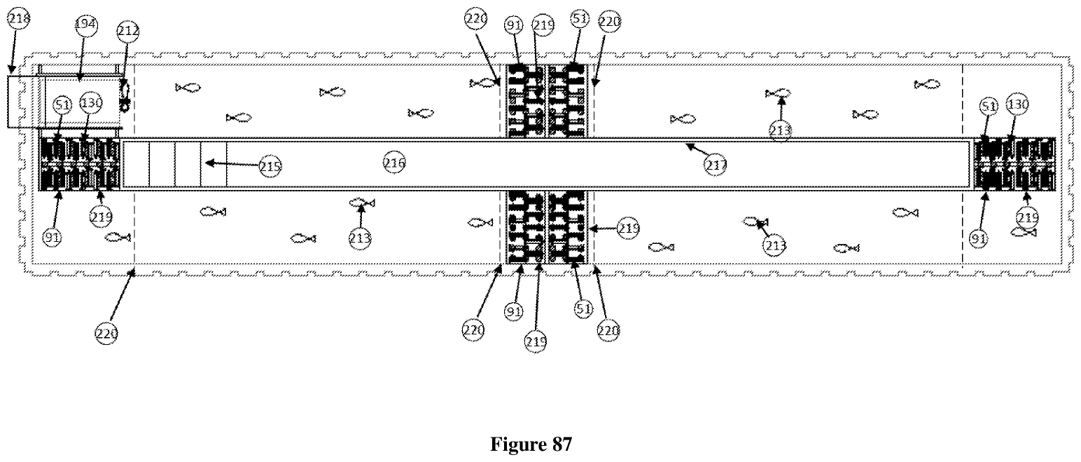

FIG. 80 shows a moving filter belt to filter water prior to treatment, which has the ability to replace a reverse osmosis membrane. Water is pumped into the moving belt and exits as close as 1 mm to the belt. The continuous moving belt will preferably carry any salt/sodium away from the water pressured area. Any salt or debris rotate around the drum at each end the pass over the first of two belt cleaners 195 is activated as the debris or salt is on the underside of the belt high-pressure water or air or blow any salt or debris into the catchment chamber 195 and exit through waste pipe 188. The number of these cleaning chambers can be as low as 1 or as many as 50, whatever is required. As the belt comes around it is continuous clean and the water enters in the water-cleaning chamber 182. If the water is not 0% salt-free, it can then be passed to a second machine, which exactly looks the same except for a finer micron or finer filtered belt to remove any remaining salt. As many pieces of this equipment may be used using finer and finer systems. The advantage of this equipment over conventional reverse osmosis filtering is that no backwashing required is a constant filtering machine all water is then sent for polarization treatment. The large drums, which carry the belt may be driven by, drive sprockets 186 or have the center drive shaft.

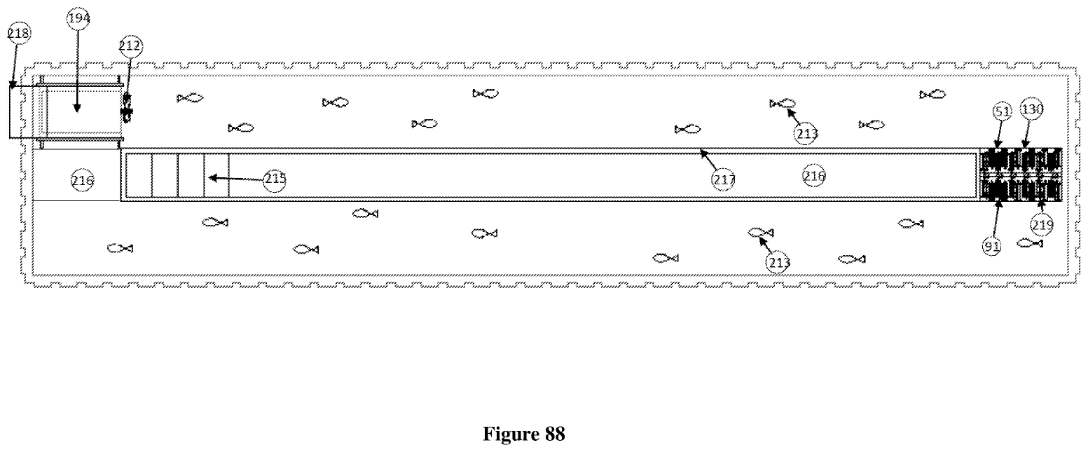

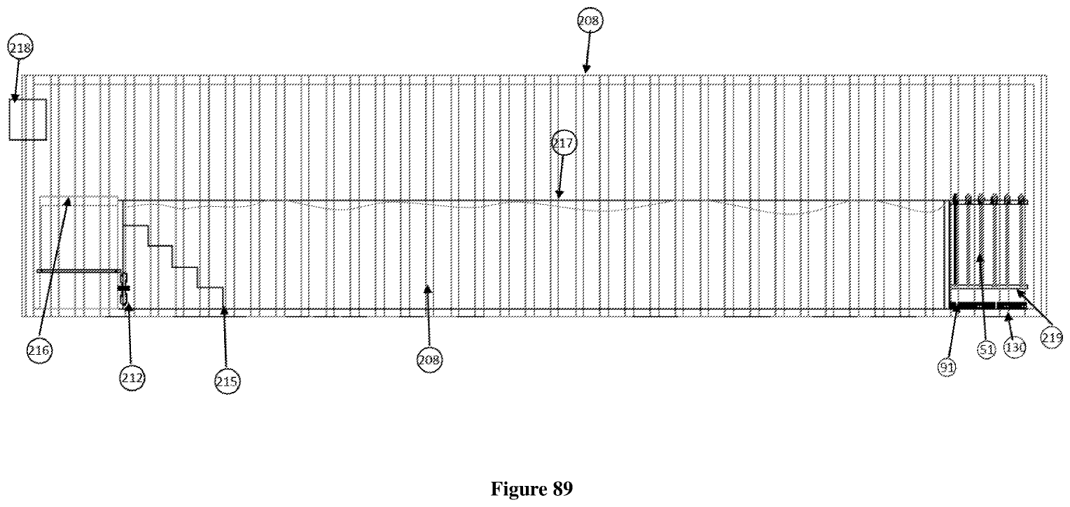

FIG. 81 is a side view of the belt filter illustrated in FIG. 80. FIG. 82 is an isometric view of the belt filter illustrated in FIG. 80 in a housing.

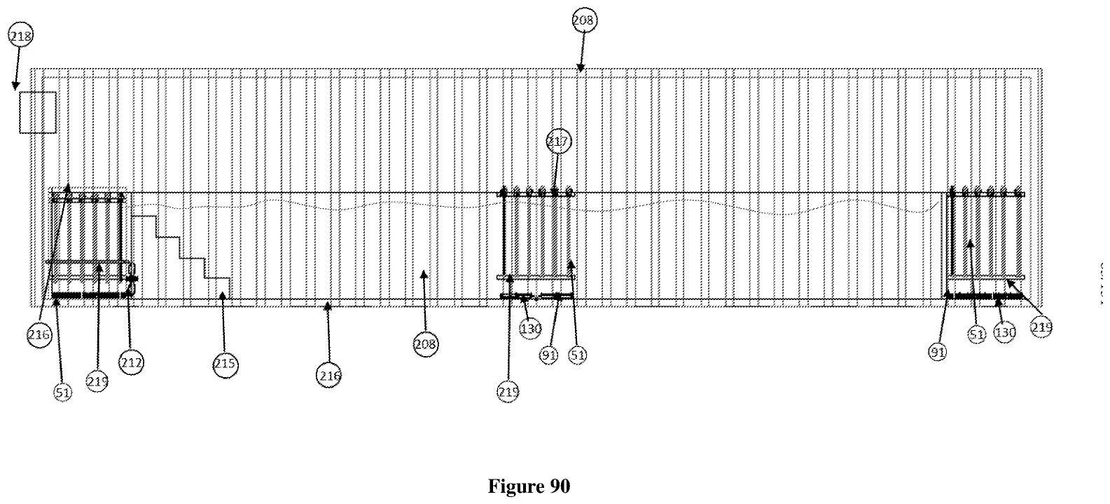

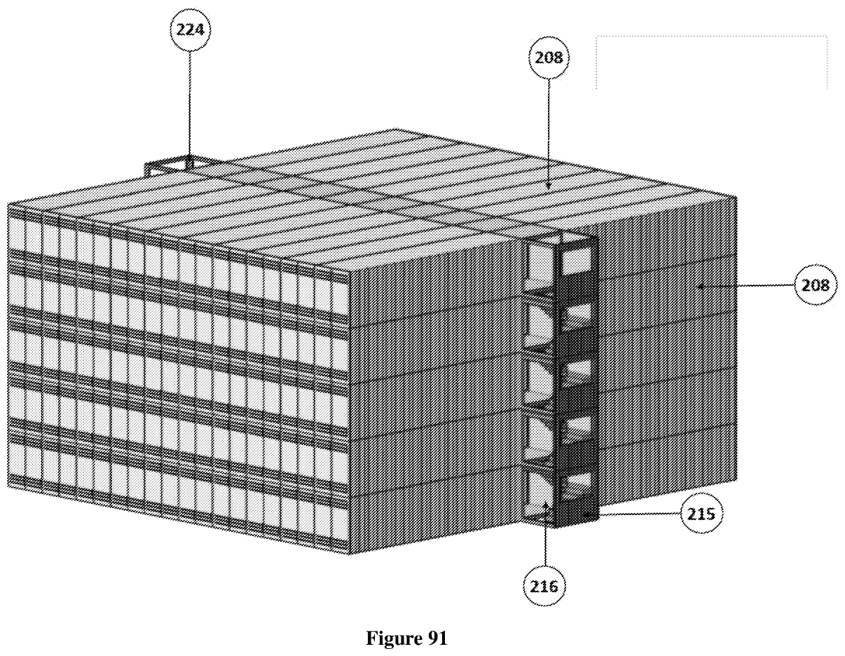

FIG. 83 is a C-shaped or U-shaped continuous treatment chamber. This is a continuous flow treatment chamber. As illustrated, the water to be treated can flow n either direction through the continuous treatment chamber. Air stone diffusion assemblies to bubble ozone through the liquid are clearly shown along with the multiple UV light assemblies. FIG. 84 is an isometric view of the reactor in FIG. 83.

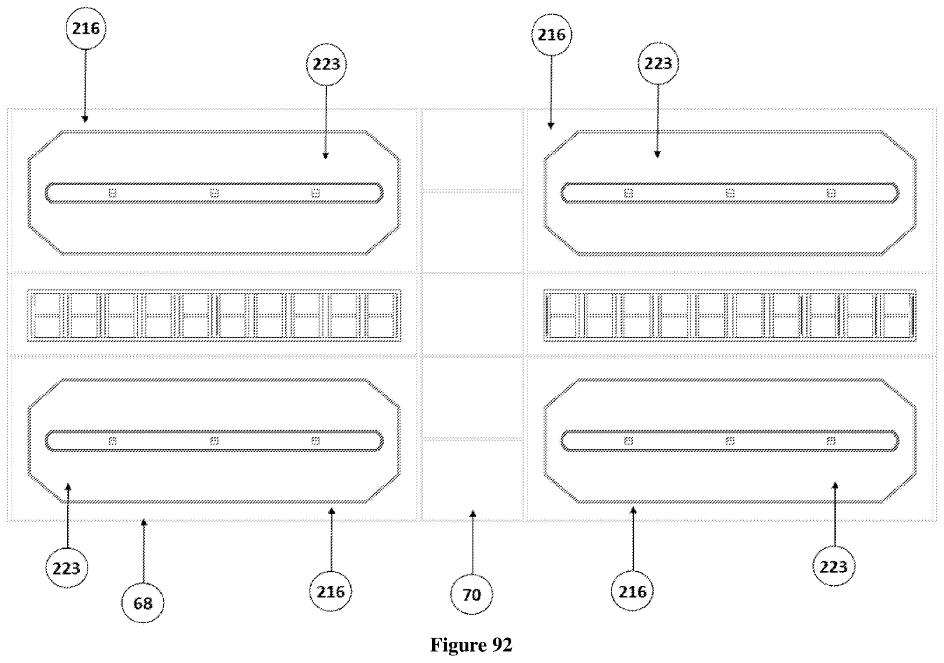

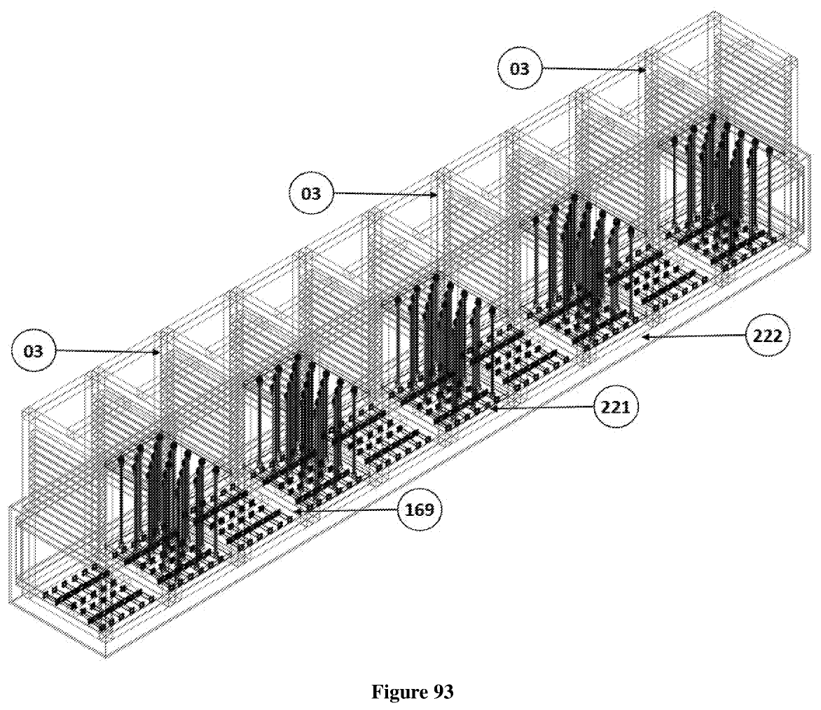

FIG. 85 shows a round continuous treatment chamber so the contaminated fluid enters through pipe 4 which has a control valve 24. The water will enter a counter clockwise and time spent in the treatment chamber will be dictated by the quality entering the system. This will typically be anywhere between 1 and 3 minutes before exiting at the pipe 3. This system can be operated at the center and therefore can be built like a tank with a center opening or side opening. Air stone ozone bubblers are clearly shown along with the multiple UV light assemblies. FIG. 86 is a side view of the embodiment illustrated in FIG. 85.

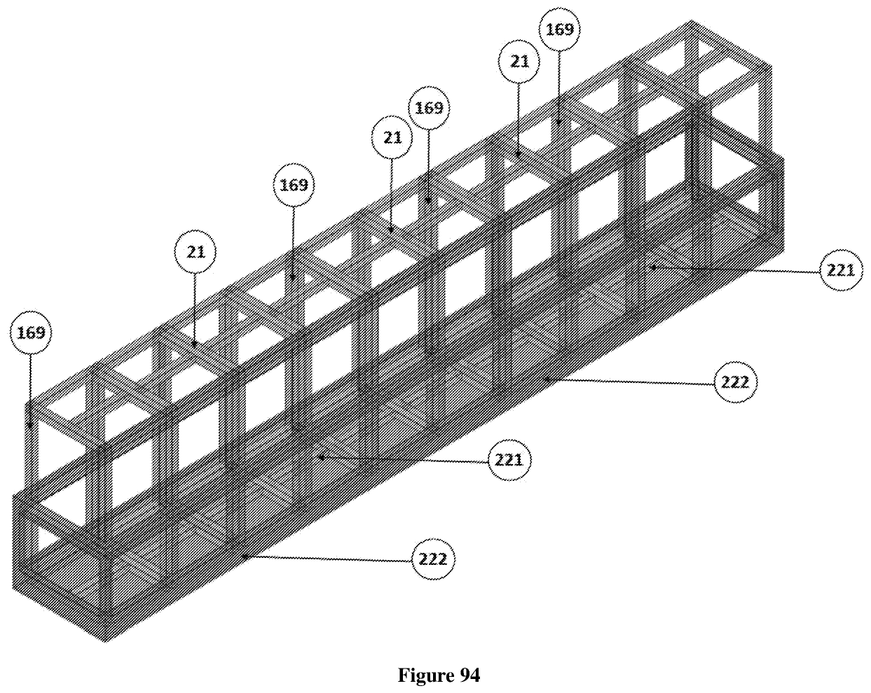

FIG. 87 s a top view of a 40 ft container embodiment fitted with internal surrounding water tanks with a walkway down the middle and four treatment chambers with ozone air stone bubblers and UV light sources. This system will operate to lower toxicity is the water which will kill fish and also eliminate ammonia which is the main killer of fish in aquaculture systems. This is normally done bacterially, and the bacteria convert the ammonia to nitrite and then to nitrate. The system of the present invention, while removing the ammonia can also eliminate ammonia and therefore, the nitrite and nitrate are not formed. These processes within the tank can be carried out externally and the water returned to the tank if required. This system can be temperature controlled by air conditioners or chilling or heating the water.

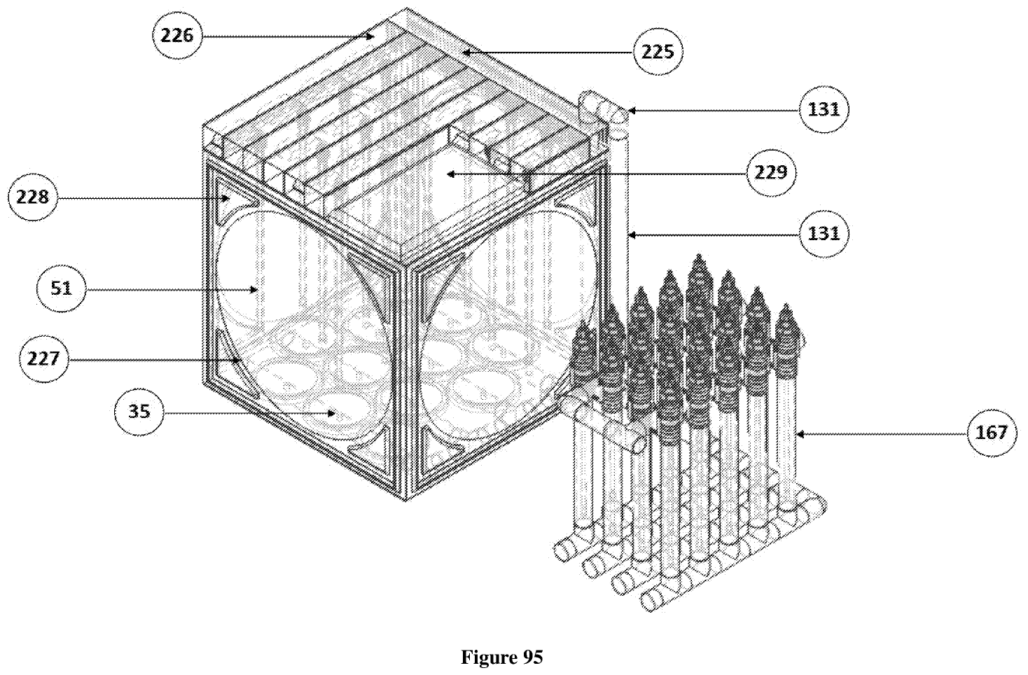

FIG. 88 is a top view of the configuration shown in FIG. 87 with a single treatment station according to an embodiment of the present invention. Also shown in FIGS. 87 and 88 is a propeller 202 which pushes the water around the tank forcing it to past through the treatment chamber(s).

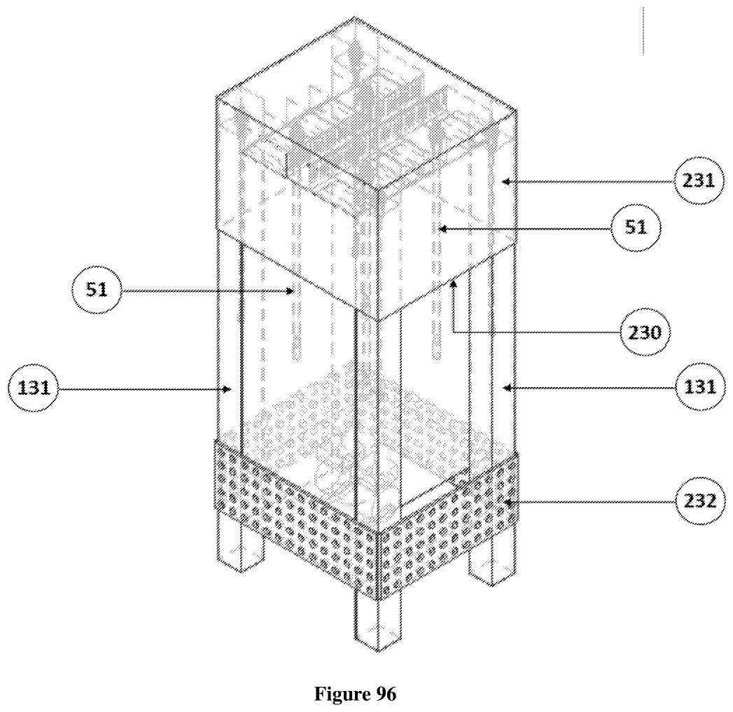

FIG. 89 is a side view of a 40 ft container converted to a temperature control aquaculture system which has temperature control 218 with treatment chamber 219 and the propeller 212 which drives the water around the system forcing it to the treatment chamber 219.

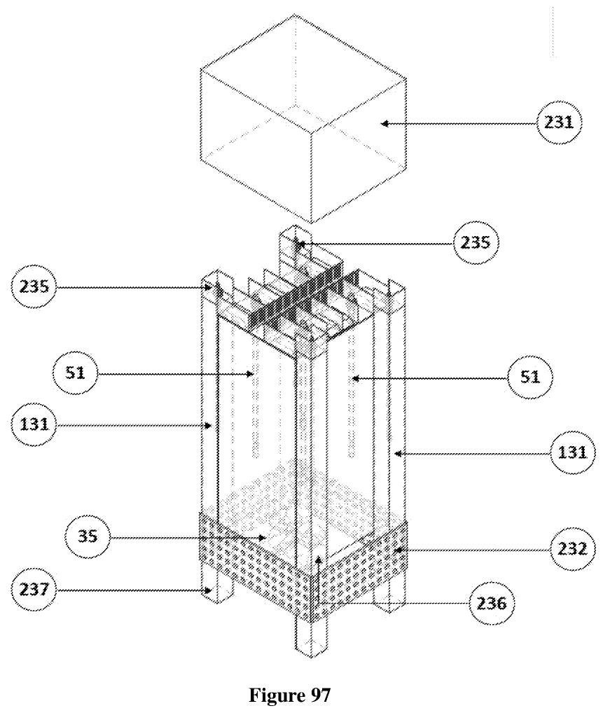

FIG. 90 is a side view of the embodiment illustrated in FIG. 87 with a multiple treatment chambers which also create high water oxygenation to increase fish stocking density and showing the steps 215 leading down to the walkway 216.

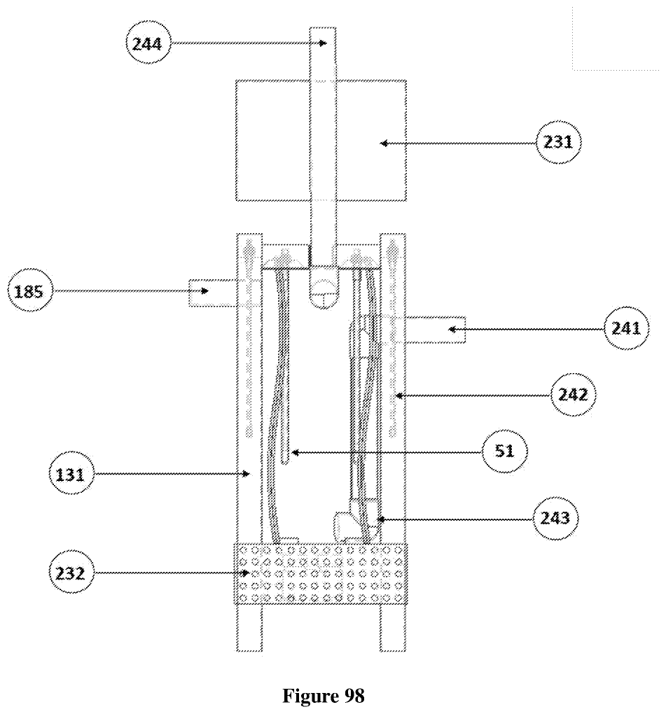

FIG. 91 shows a block of 100 containerized units in five layers of 20 containers holding aquaculture systems including fish and 10 central containers in each layer holding water treatment systems. At one end the 10 ft Containers which create walkways steps and an industrial lift. At the other end, all the containers are bolted together for a solid structure.

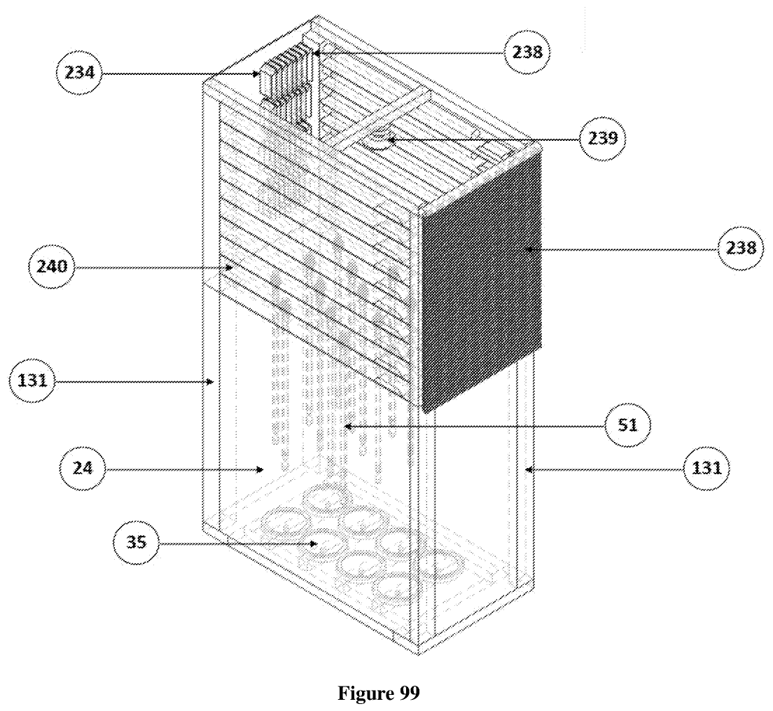

FIG. 92 is an overhead view of two recirculating fish tanks, each built into two containers with support post in the middle and trusses to give the container strength where the internal walls have been removed. Each tank sits astride a single container with the water treatment system for the removal of ammonia and other bacteria along with managing oxygen levels. In the centre of the design, are 10 ft containers which becomes walkways on each level to allow access to the recirculating fish tanks and water treatment system.

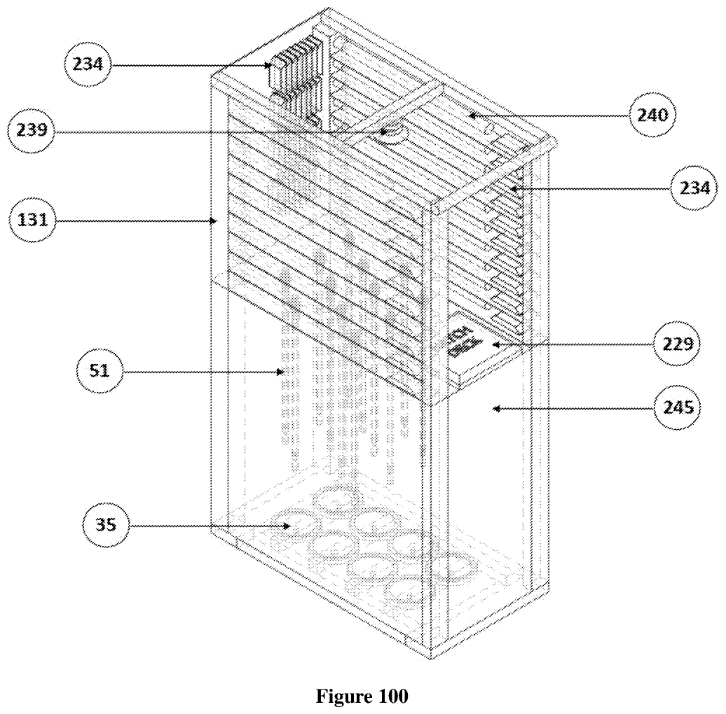

FIG. 93 shows a water treatment system comprising a base and an ozone bubbler assembly situated horizontally. Each alternate chamber has ozone producing UV tubes which are supplied with compressed air which is delivered to the base of the system through structural tubes and then to ozone air stones or venturis. UV lights are situated vertically or horizontally to intersect and react with the rising bubbles to create the treatment area.

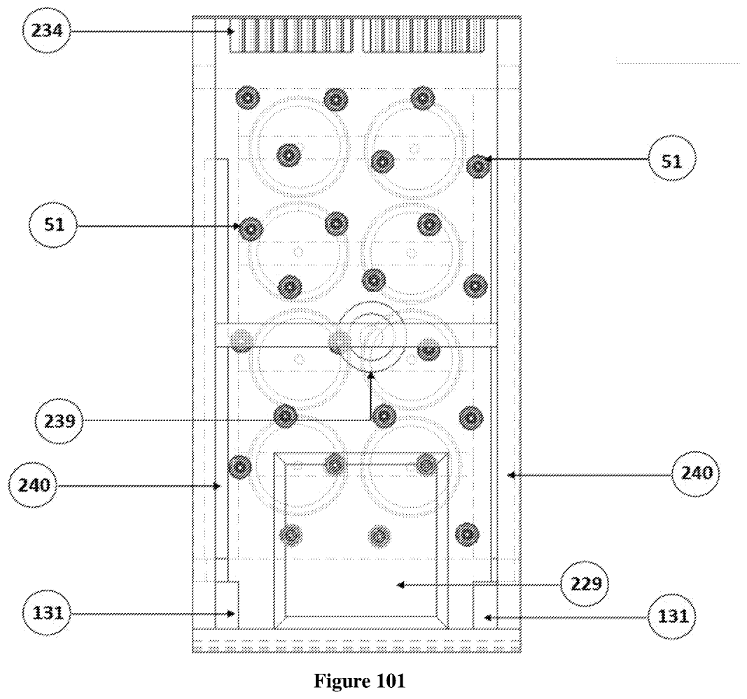

FIG. 94 shows the base frame of the embodiment shown in FIG. 93 which holds the water or liquid. Also shown is the wastewater and gas removal frame 222 at the base and the internal frame which holds the ozone producing tubes, air stones and diffusers at the base. Both frames are separate and both frames have different purposes.

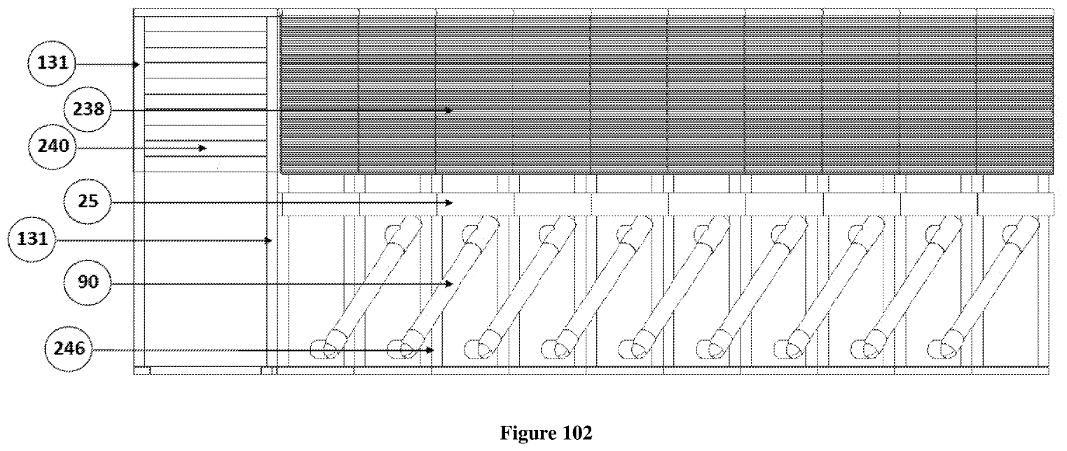

FIG. 95 is an exploded view of a treatment chamber for treating water or fluids with ozone production unit 167.

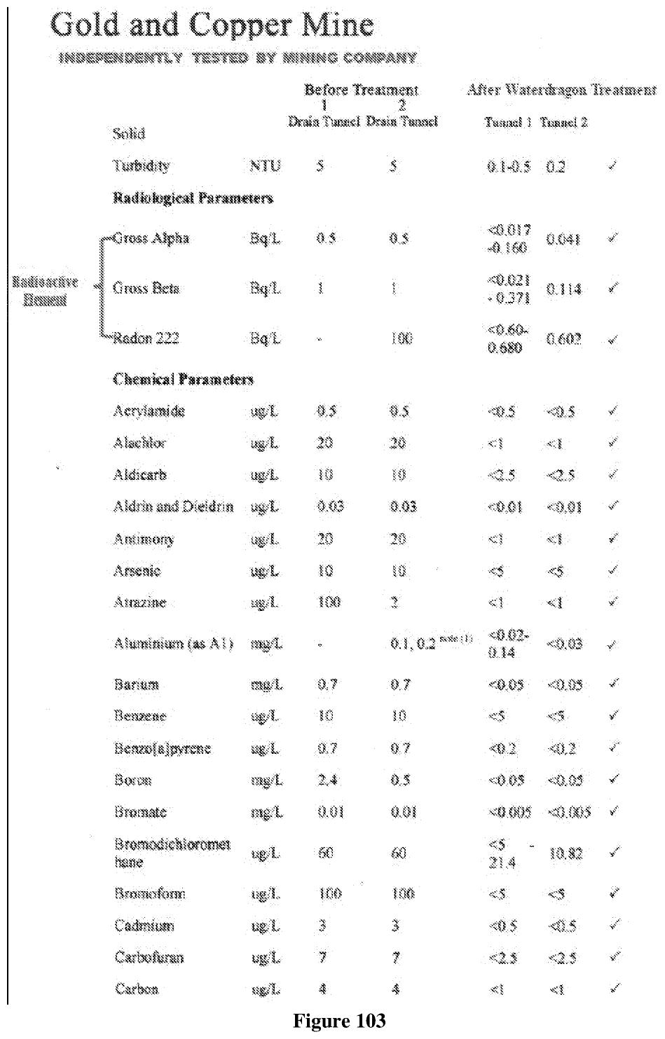

FIG. 96 shows an outdoor or external treatment chamber suitable for sewage or swimming pools, drinking water or any other application for treating water or fluids. The system is showing in a lock operating position. Water enters from the top of the treatment chamber and exits 20% down from the top when extracting the water from the bottom of the chamber and exits toward to the top of the chamber at the lower level of the input chamber. This ensures that the water entering from the treatment chamber will have to work its way down through the chamber and be treated by the treatment process.

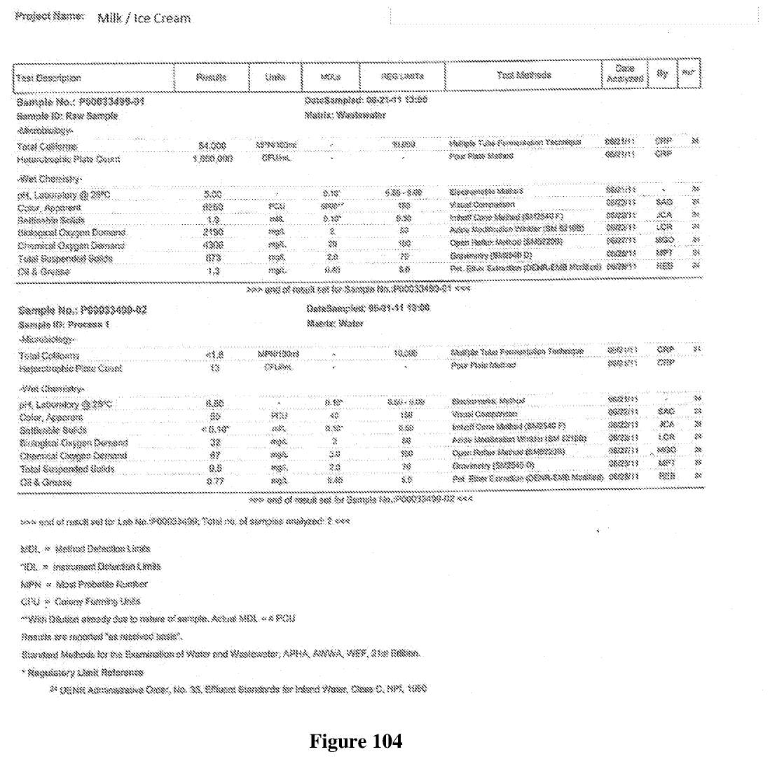

FIG. 97 shows the waterproof external or internal treatment chamber with air pump underneath which pushes air in the four external structural tubes with 180 nanometer ozone producing lamps. This can be multiple or singular and the air passing from the bottom to the top over the UV light waves will produce ozone which is then injected to the water inside the chamber. One or more of the structural tubes can be used to exhaust gas from the chamber if required. The ozone gas exhausting from the chamber can be mixed with compress air produce underneath the chamber to create quick destruction of the ozone back into the air.

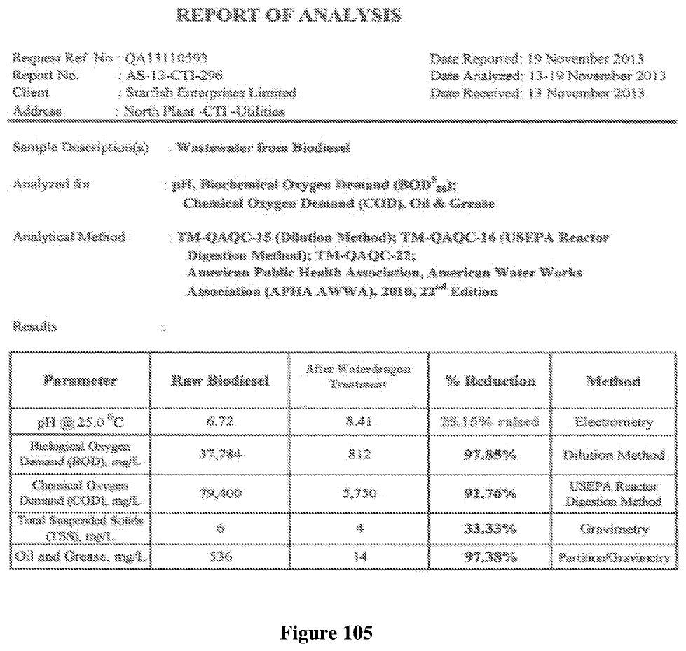

FIG. 98 shows the input or polluted water entry pipe at the top left where the water to be treated enters. On the right-hand side on the top where the clean water that has been treated exist the water. The height of this pipe controls the water level within the system. Water enters the exit pipe from the base of the system internally or externally and moves upwards to the outlet of the treated water. The height of the clean water exist pipe can be moved to control the level within the chamber to any desired position. The pipe on the very top of the system is for exhaust gases for used ozone to exist in the reaction chamber. Air may be also added to this pipe to a quick breakdown of the ozone gases back to clean air. An ultraviolet bulb of 254 nanometers or similar may also be added to speed up the breakdown of the ozone gases. The final product will be clean germ-free air.

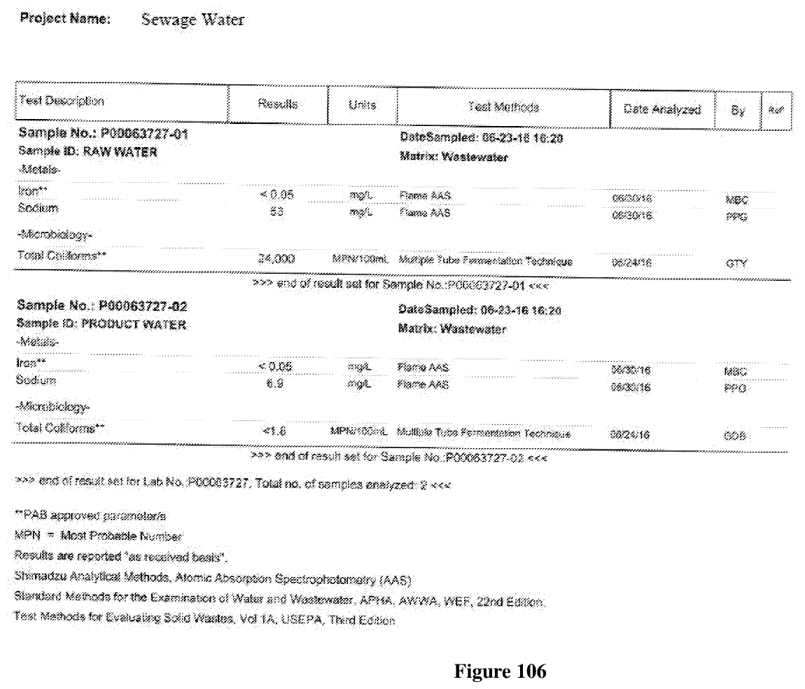

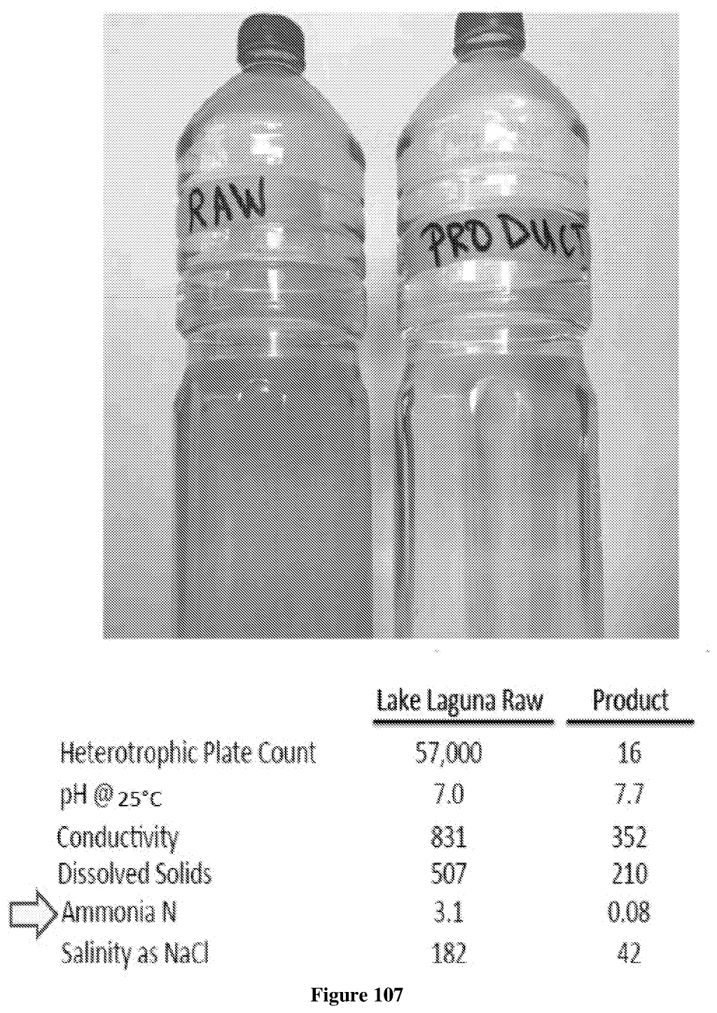

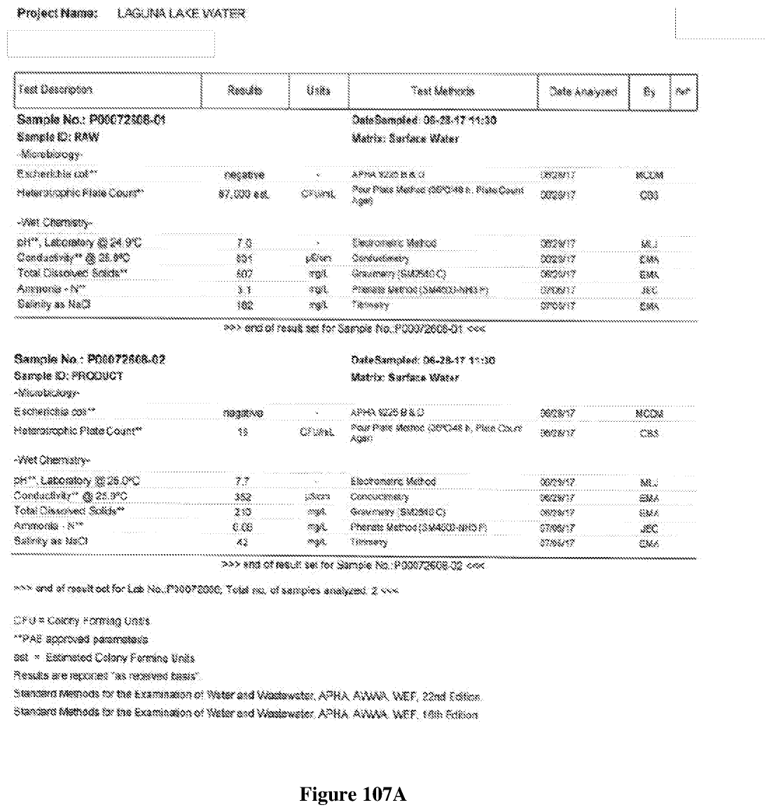

FIG. 99 is a larger scale version of the configuration illustrated in FIG. 96. FIG. 100 shows the configuration illustrated in FIG. 99 with the shutter removed. FIG. 101 is a top view of the configuration illustrated in FIG. 100.

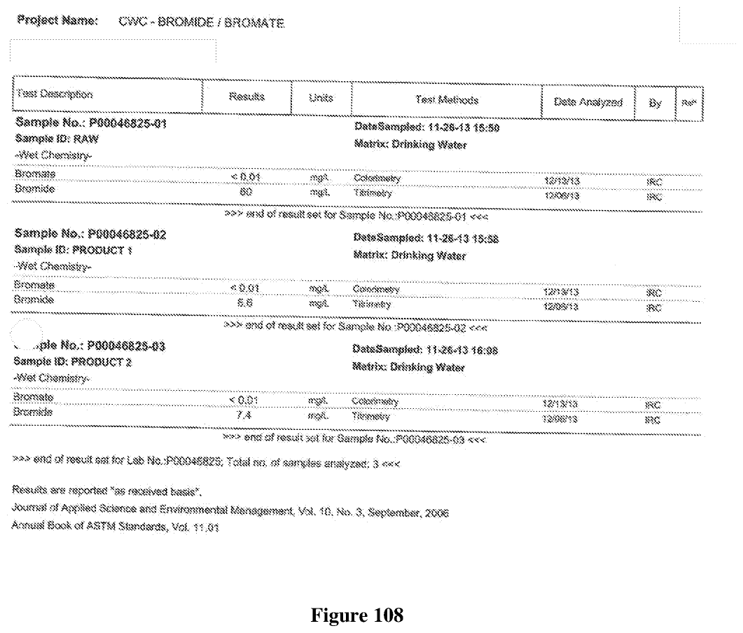

FIG. 102 shows polarization reactors individually built and joined into multiple systems with water flows between from top to bottom to bottom to top. Also clearly showing is the vacuum, water, and ozone gases which can overflow into the waste pipe which can be free-flowing or by adding additional compress air flowing over pipes from the chamber which creates venturi. As additional air passes at speed, it will create a vacuum in the chamber if required. At the end of this tube, a U-bend can separate the water or fluids and the gases which can go up as the water goes down. This Figure also shows the roller shutters which turn all electrical areas into a sealed electrical box. This configuration can still allow ventilation from the top. Security can be achieved via a stainless steel welder mesh type product which will stop access from the chamber. This system can be placed into a shipping container for security made up of containers. The angle fluid transfer pipe for one place to another may be fitted with various wavelengths of ultraviolet to create additional reactions and destruction of viruses, bacteria, heavy metals or chemicals.

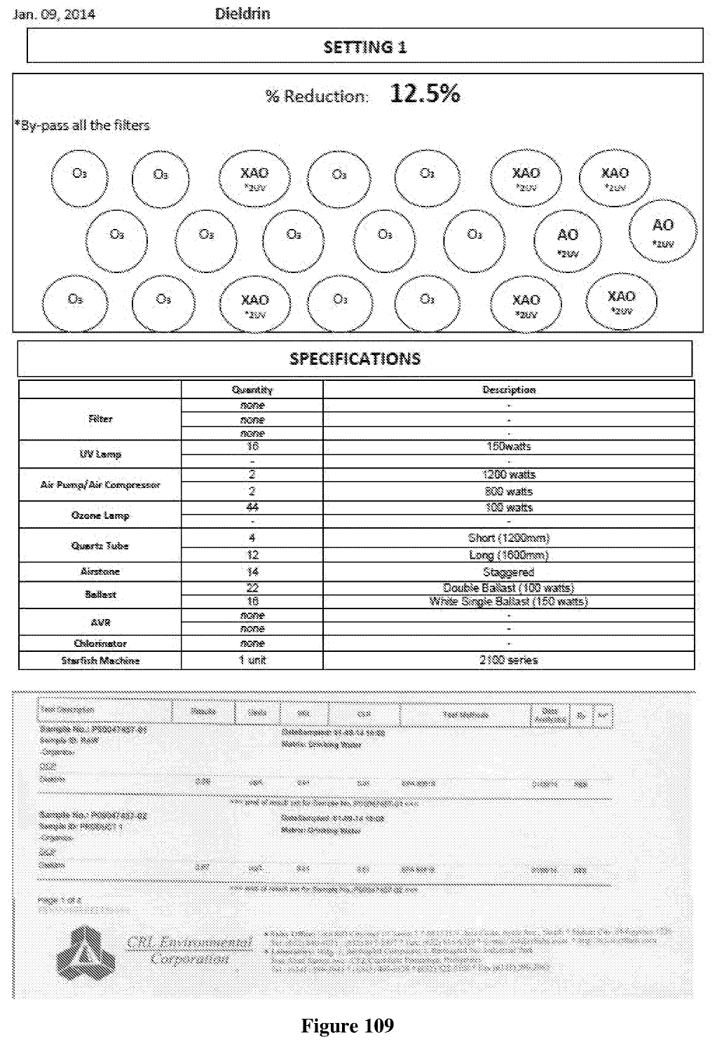

FIG. 103 shows the laboratory results for treatment of water flowing from an underground gold and copper mine by an embodiment of the present invention.

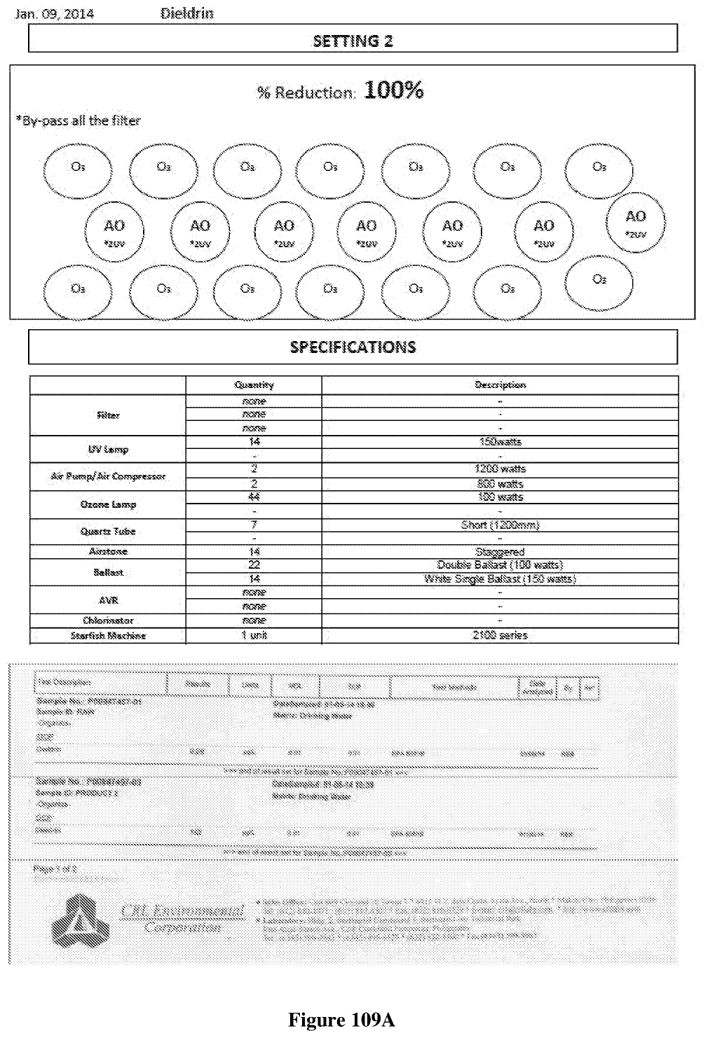

FIG. 104 shows the laboratory testing of milk waste from an ice cream factory treated by an embodiment of the present invention.

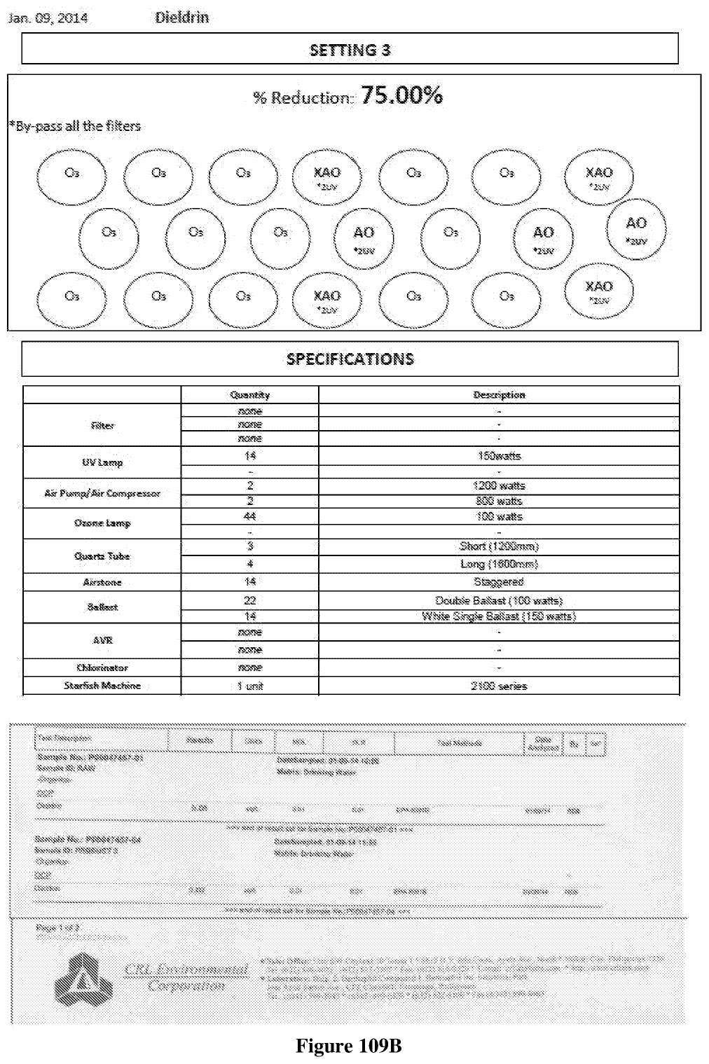

FIG. 105 shows the testing of biodiesel treated by an embodiment of the present invention.

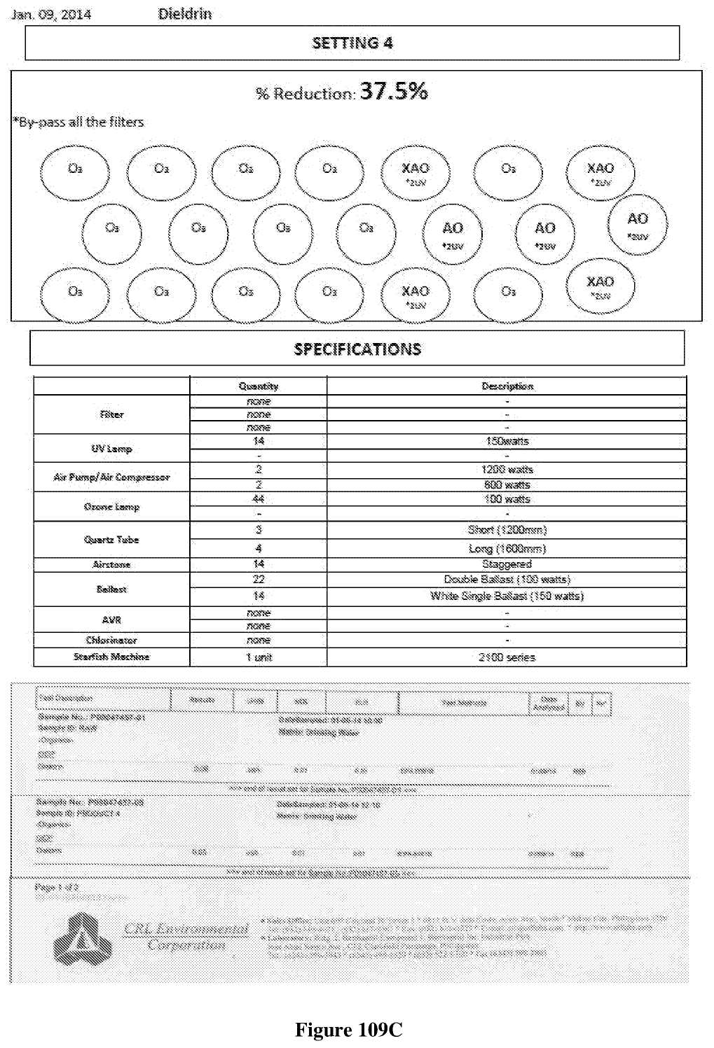

FIG. 106 shows the testing of sewage water treated by an embodiment of the present invention.

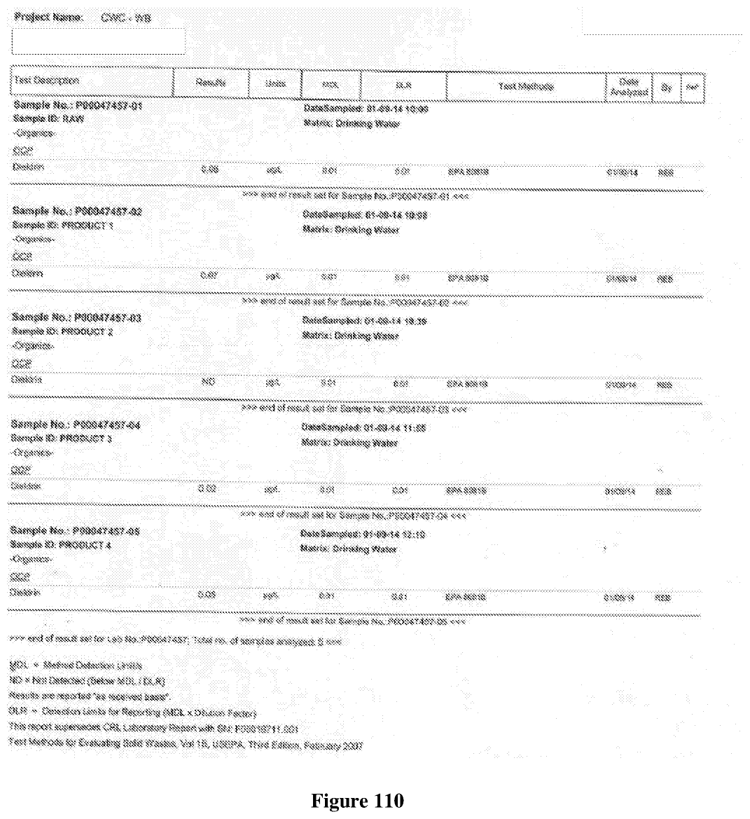

FIG. 107 shows a comparison of lake water before and after treatment by an embodiment of the present invention.

FIG. 107A shows the test results of the water in FIG. 107.

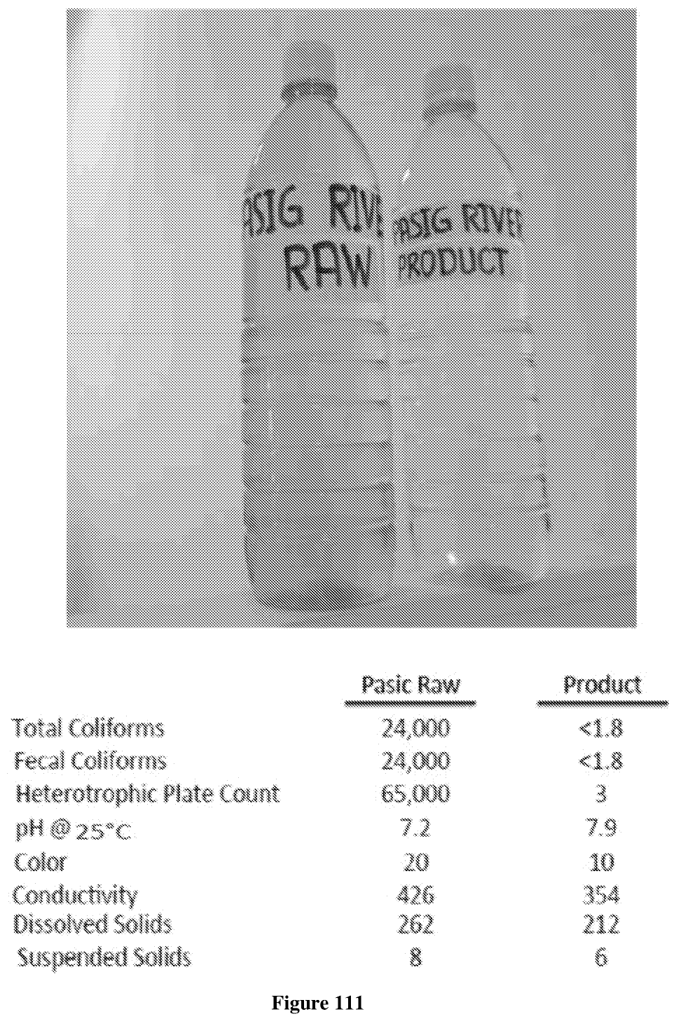

FIG. 108 shows the test results for water including bromide after treatment by an embodiment of the present invention.

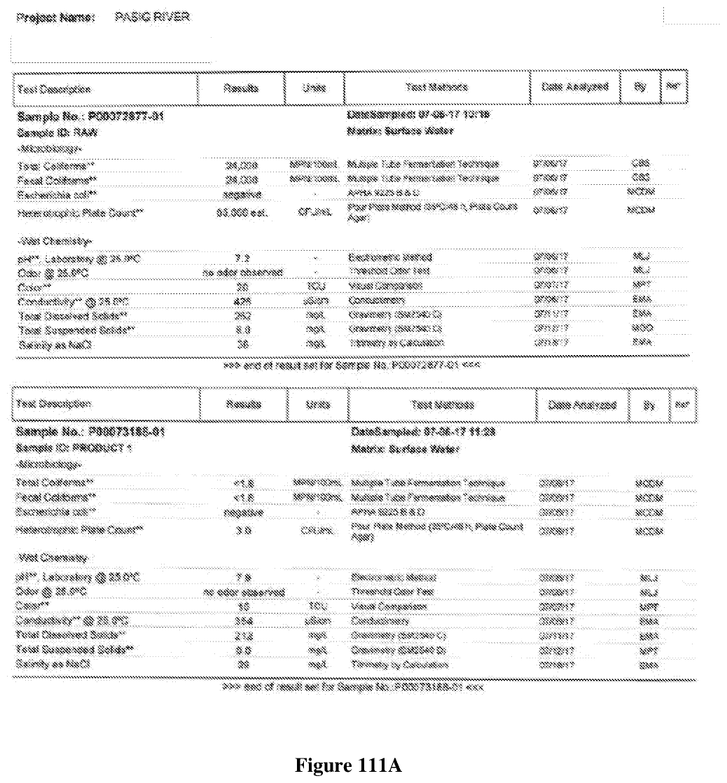

FIG. 109 shows the effect of one configuration of the present invention including 8 UV lights on the treatment of dieldrin. In the diagram shown in FIG. 109 each circle represents a treatment chamber. Fluid travels from the left to the right. Therefore, the fluid starts in the two chambers labelled with O₃ on the far left, then fluid travels from both chambers into the next chamber labelled O₃, then the fluid splits into the next two chambers labelled O₃. The fluid is consecutively combined then divided as shown in FIG. 109. The chambers labelled O₃ are ozone treatment chambers, where ozone is bubbled (using a microbubbler as a dispersion assembly) without use of UV lights through the treatment chamber. The chambers labelled AO (or advanced oxidation) are ultraviolet light treatment chambers (where ozone is not bubbled through the chamber), but residual ozone in the fluid stream is treated with UV light (at a wavelength of about 253 nm). The chambers labelled XAO (or extreme advanced oxidation) are treatment chambers in which both ozone is bubbled (using a microbubbler as a dispersion assembly) and with use of multiple UV lights (at a wavelength of about 253 nm). The ozone used in the system is supplied from an ozone generation chamber, in which air is pumped into the chamber before the air is irradiated with a plurality of ultraviolet lights at a wavelength of about 180 nm to produce ozone.

FIG. 109A shows the effect of one configuration of the present invention on the treatment of dieldrin.

FIG. 109B shows the effect of one configuration of the present invention on the treatment of dieldrin.

FIG. 109C shows the effect of one configuration of the present invention on the treatment of dieldrin.

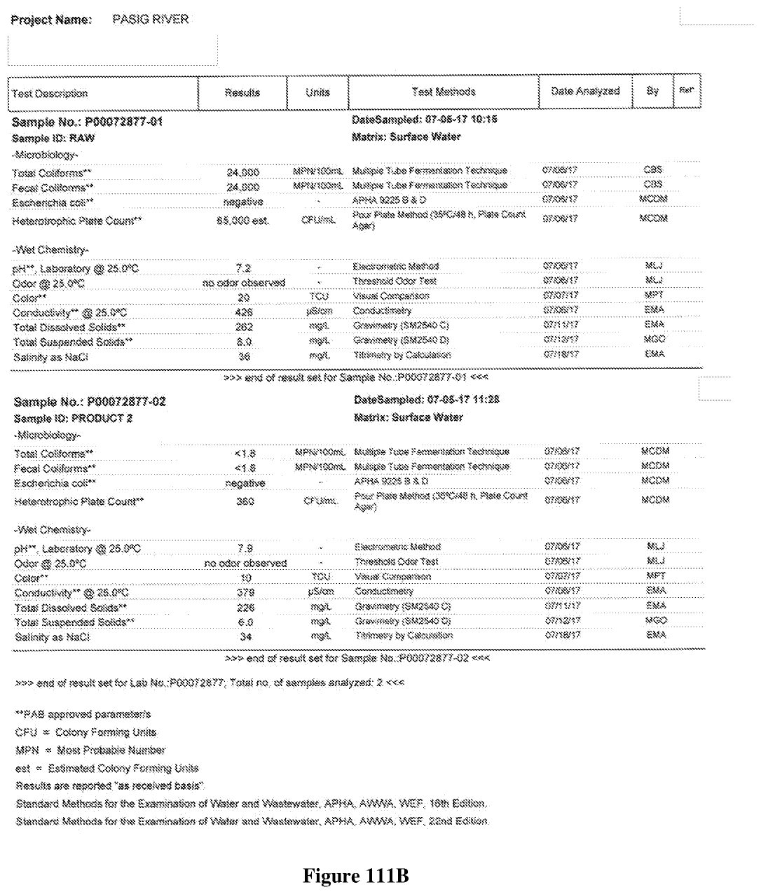

FIG. 110 shows the test results of treatment of water including dieldrin by one configuration of the present invention.

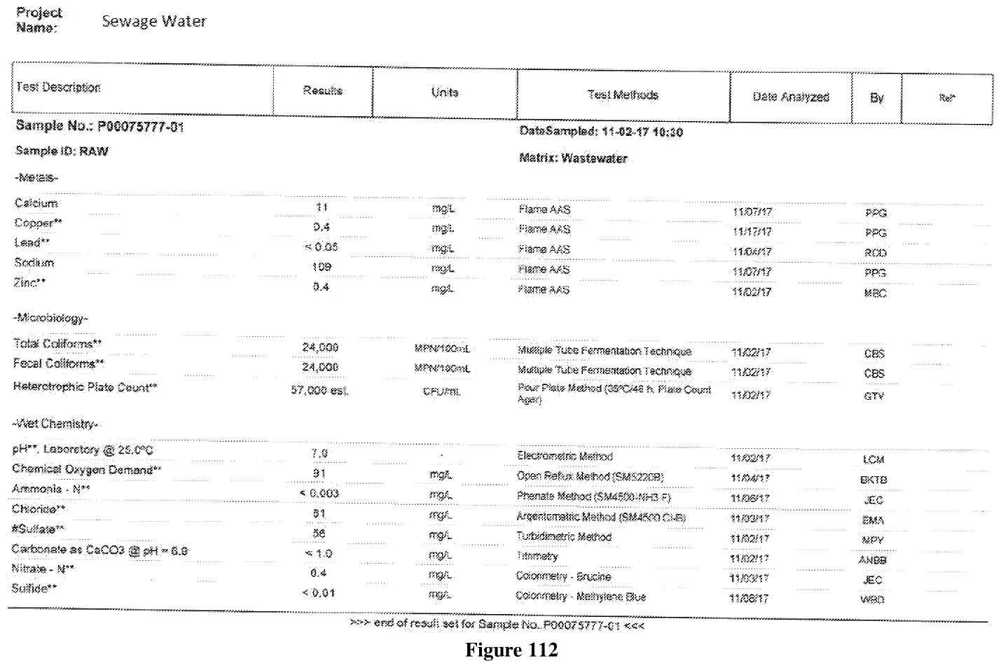

FIG. 111 shows one set of results of the system of the present invention on water drawn from the Pasig River.

FIG. 111A tabulates one set of results of the system of the present invention on water drawn from the Pasig River.

FIG. 111B tabulates a second set of results of the system of the present invention on water drawn from the Pasig River.

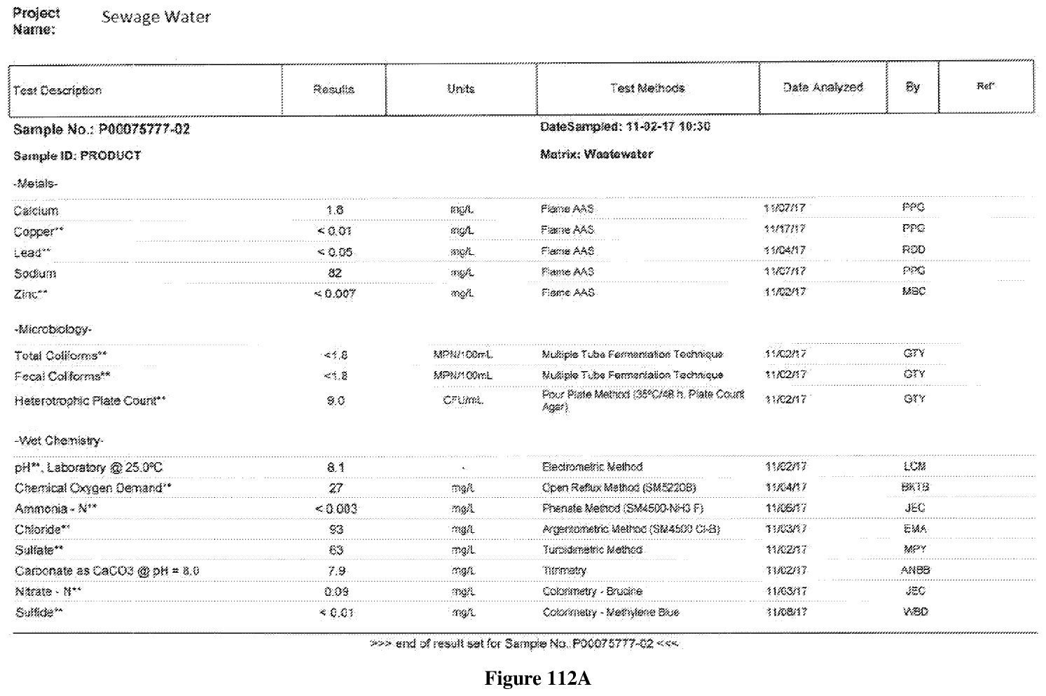

FIG. 112 shows a first set of results in the treatment of raw water in sewage water by an embodiment of the present invention.

FIG. 112A shows a second set of results in the treatment of raw water in sewage water by an embodiment of the present invention.

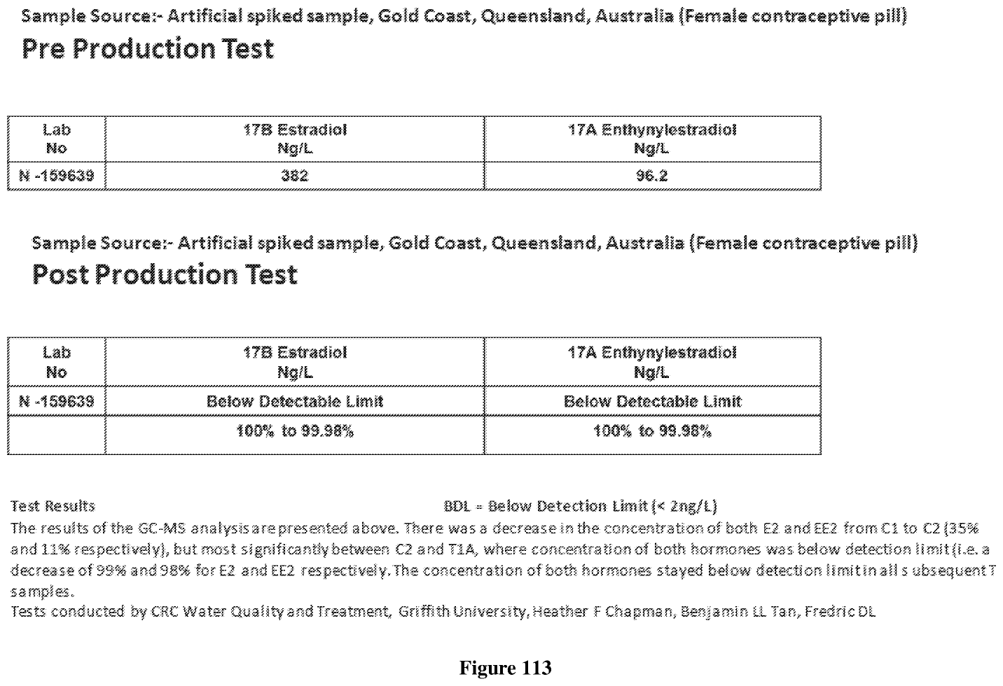

FIG. 113 shows the results of a natural and synthetic hormone removal by an embodiment of the present invention.

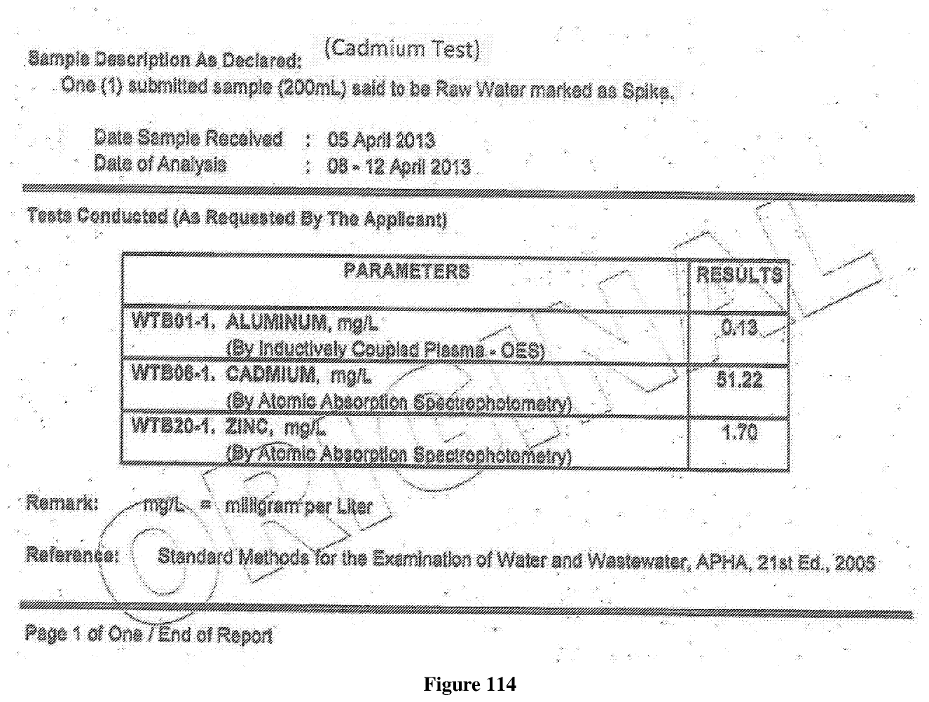

FIG. 114 shows the results of treatment of a first sample with very high level of cadmium, aluminium and zinc by an embodiment of the present invention.

FIG. 114A shows the results of treatment of a second sample with very high level of cadmium, aluminium and zinc by an embodiment of the present invention.

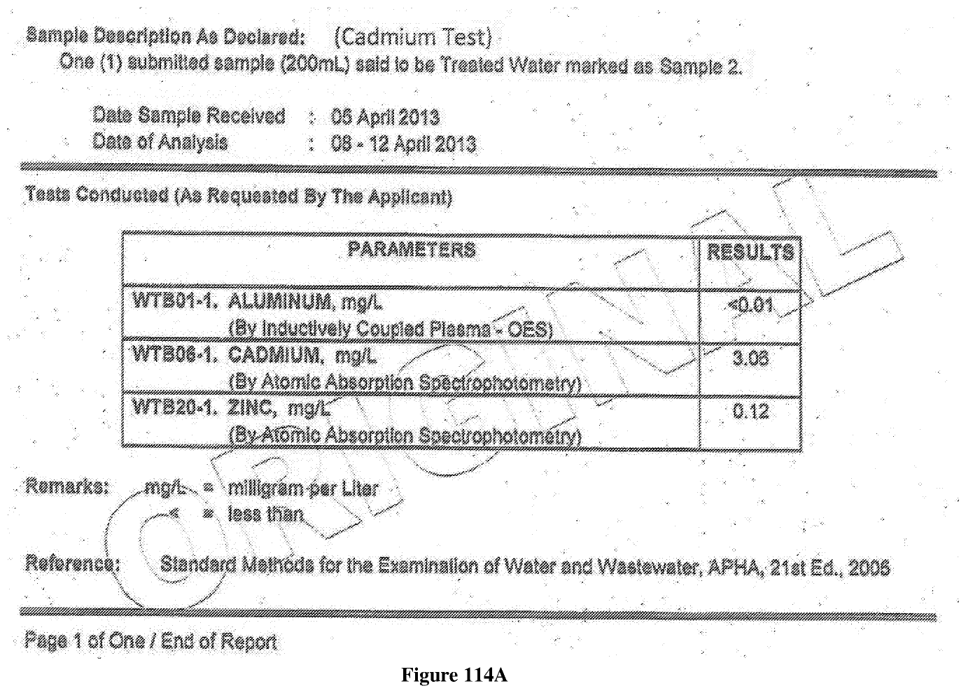

FIG. 115 shows the testing of biodiesel treated by an embodiment of the present invention.

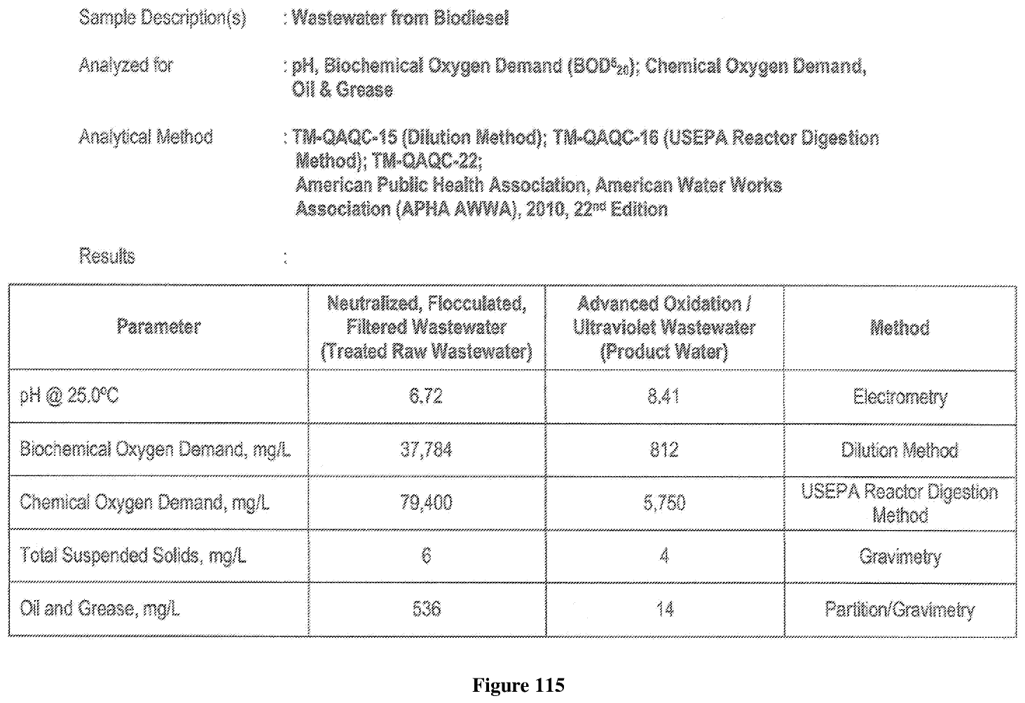

FIG. 116 is a comparison of production of ozone by corona discharge compared to the production method of an embodiment of the present invention.

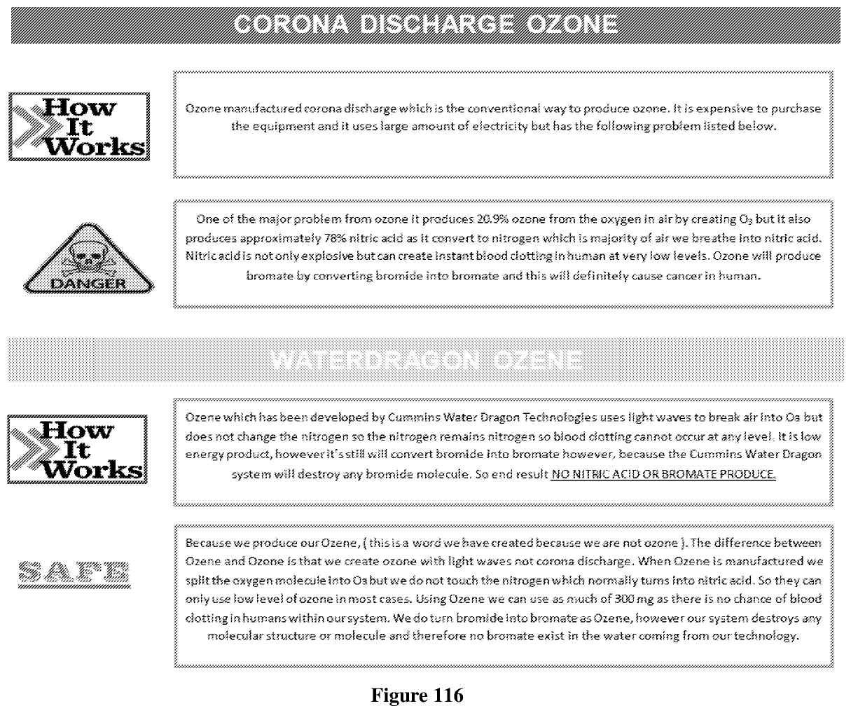

FIG. 117 is a comparison of ozone generated with the system of the present invention and not functioning and when functioning.

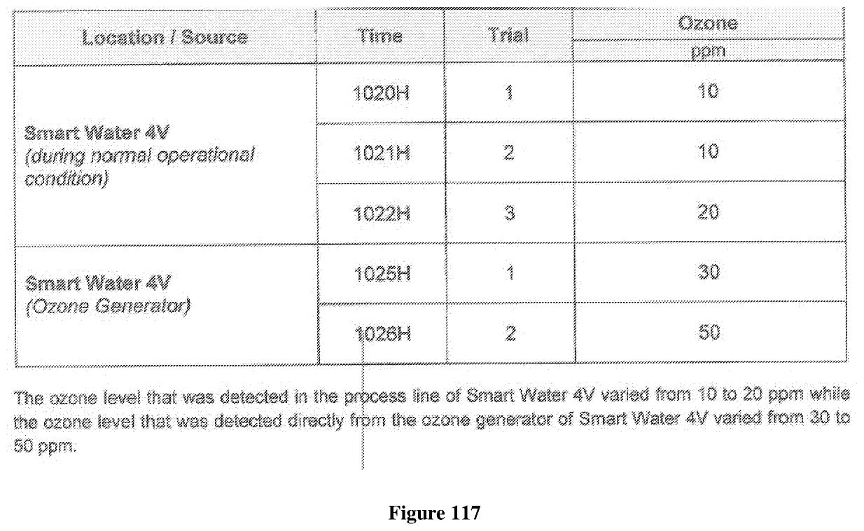

FIG. 118 shows the result of a seawater test treated by an embodiment of the present invention.

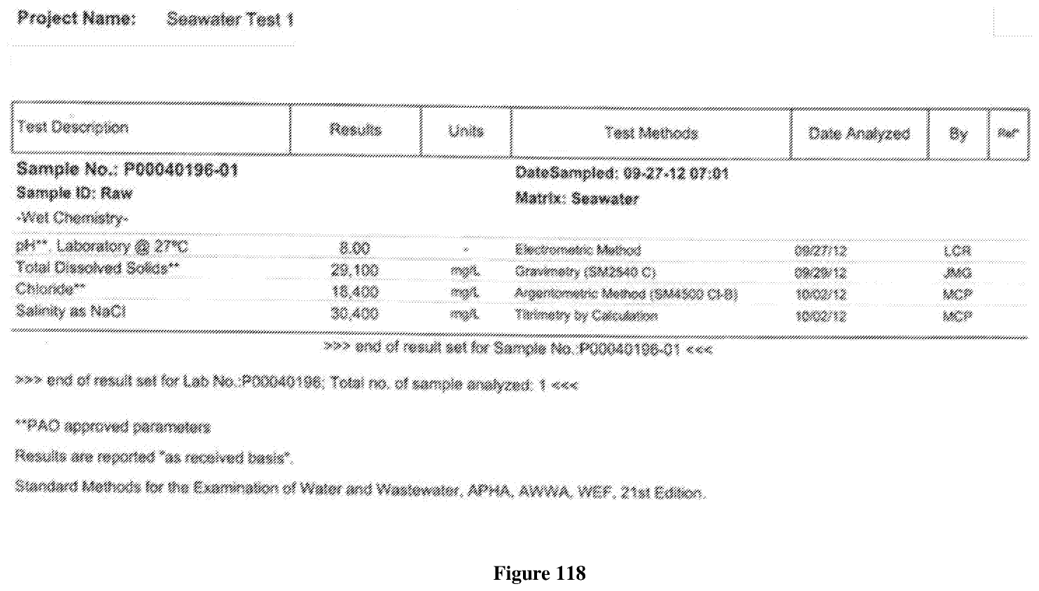

FIG. 119 is a photograph of a treatment area of a preferred embodiment viewed through a high-quality camera lens with UV filter.

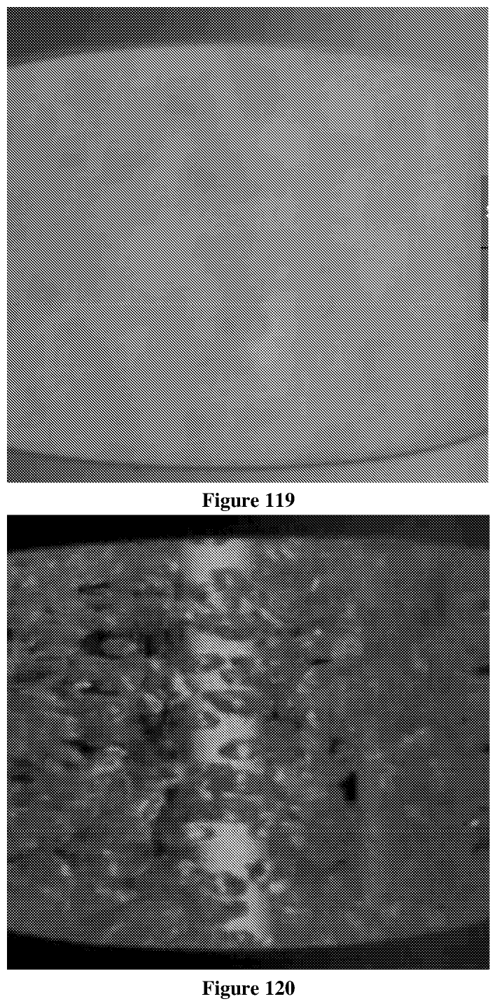

FIG. 120 is the same chamber illustrated in FIG. 119 viewed by the naked eye showing the energized ozone bubbles.

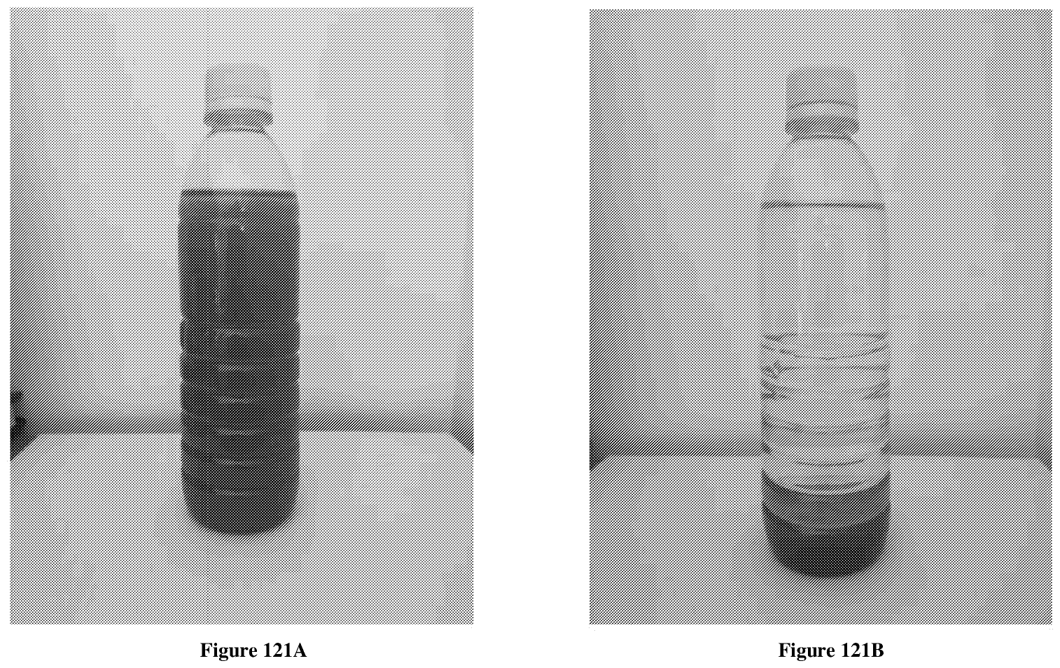

FIG. 121A is a photograph showing copper and nickel and other metals as suspended solids in a water sample.

FIG. 121B is a photograph showing the copper and nickel precipitated out after treatment from an embodiment of the present invention.

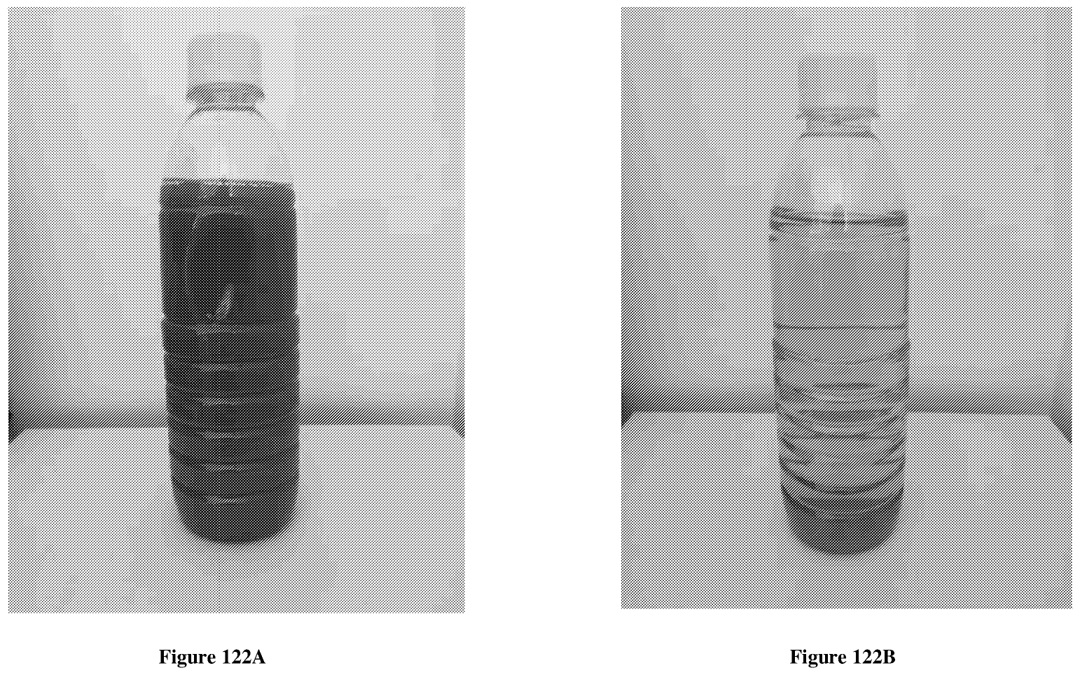

FIG. 122A is a photograph showing iron sulphate as a suspended solid in a mine water sample.

FIG. 122B is a photograph showing iron sulphate precipitated out after treatment from an embodiment of the present invention.

The examples illustrated in FIGS. 103 to 122B highlight:
Reduction in radioactive elements and almost all chemicals
Coliforms & plate counts. Reduction in BOD and COD and colour reduction in Example 2
Rising pH without additives, reduction in BOD, COD & oil and grease (fractionated from system) Example 3
Sodium reduction, and coliform reduction in Example 4
Heterotrophic plate count without additives. Conductivity drop, ammonia and salinity reduction in Example 5
Pharmaceuticals & Contraceptives (example 9)

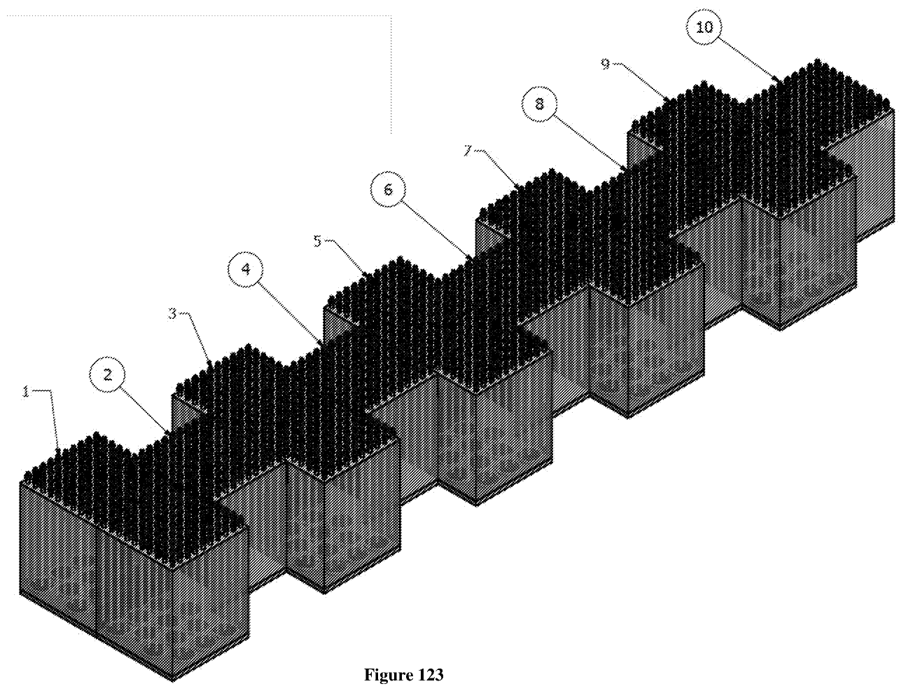

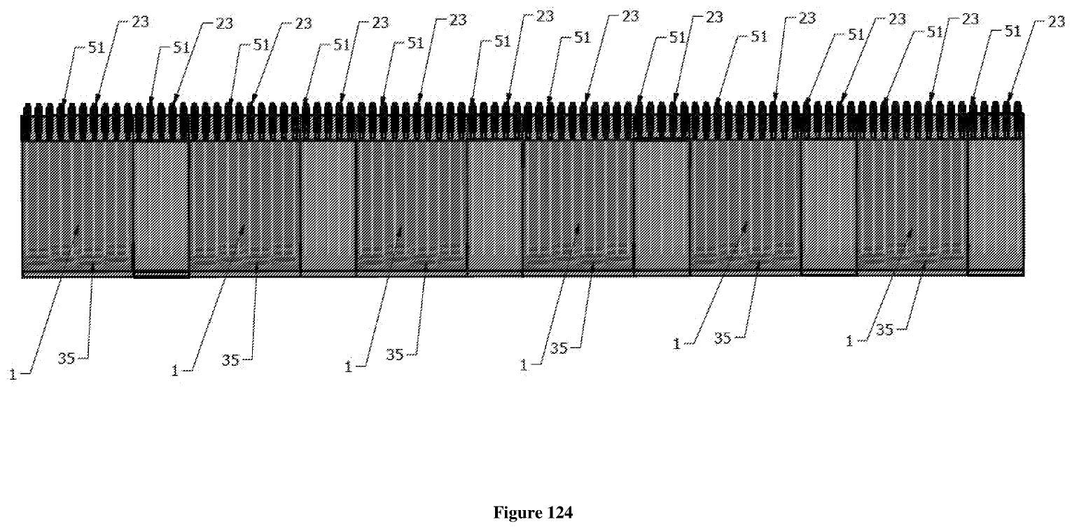

Another alternative configuration is illustrated in FIGS. 123 and 124. This system shows chambers 1,3,5,7 and 9 are twice the size of the other intervening chambers. The larger chambers are distribution chambers with microbubblers or nanobubblers, air stones and fine pore ventures or diffusers included to assist with ozone saturation of the water or fluid to be treated.

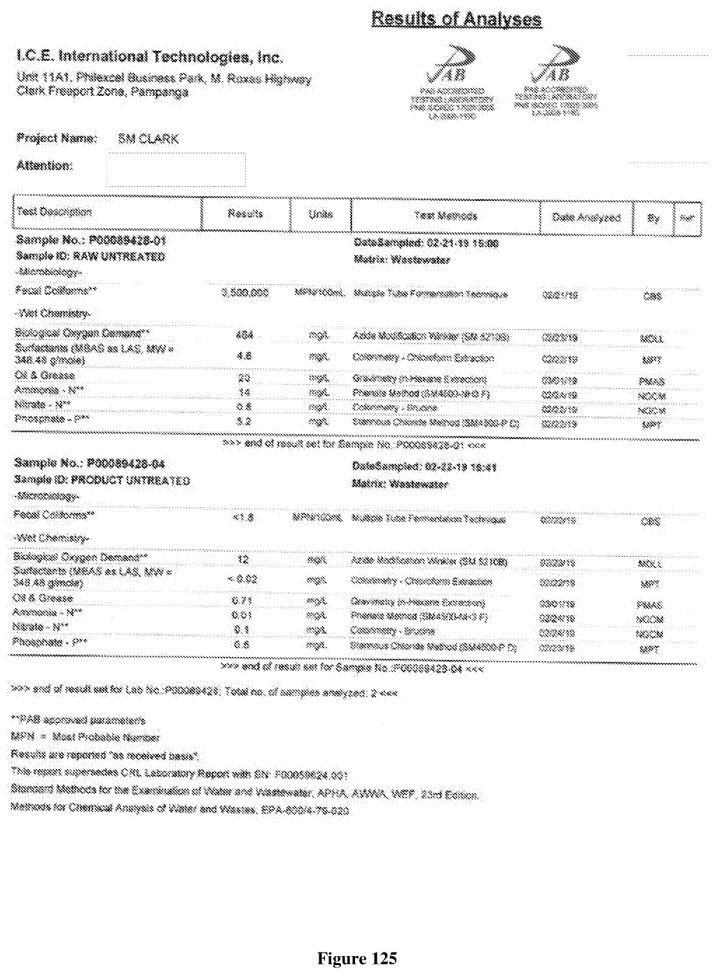

Chambers 2,4,6,8, and 10 have no ozone bubblers but have residual ozone (from the saturation in the respective preceding chamber) and UV lights treating the fluid at approximately 254 nm. This creates additional and/or differing reactions to those occurring in chambers 1,3,5,7, and 9. This configuration of the chambers created the result of the untreated sewerage shown in FIGS. 125 and 125A.

FIG. 124 shows that chambers 2,4,6,8 and 10 are half the size of the ozone distribution chambers 1,2,6,8, and 10.

UV lights are shown in all chambers in the configuration illustrated in FIGS. 123 and 124. These multiple reactors with different treatment foci in an alternating pattern are best for general use with chemical breakdown and for heavy metal destruction along with all other bacteria, viruses, BOD, and COP and the like.

FIGS. 125 and 125A show the treatment results of treating two different raw wastewater input and the resultant product streams from a system as illustrated in FIGS. 123 and 124.

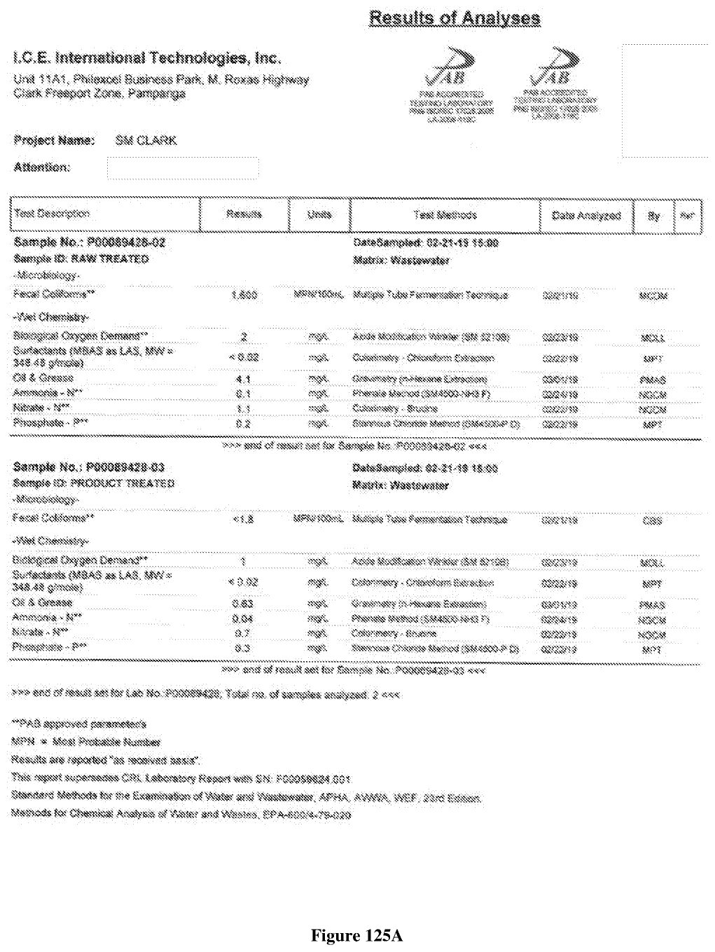

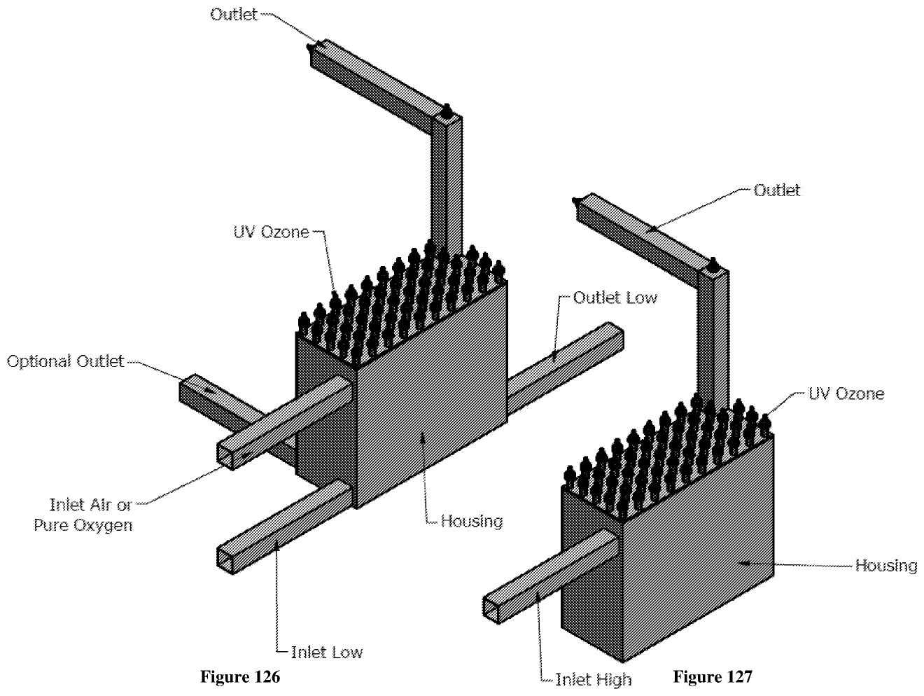

Two examples of an ozone generator which may be used in the system of the present invention are illustrated in FIGS. 126 and 127. The embodiment shown in FIG. 126 is the same as that illustrated in FIG. 127 but with an additional lower inlet and two optional outlets, one substantially opposite the lower inlet and an optional side outlet. Both configurations have a plurality of UV lights operating at an ozone formation wavelength. The UV lights are provided in a vertical orientation within an external housing. Typically, an ozone generator such as one of these examples is positioned immediately adjacent a treatment chamber to which the formed ozone is provided.

FIG. 117 shows the output ozone created in an ozone generator such as that illustrated in either FIG. 126 or FIG. 127.

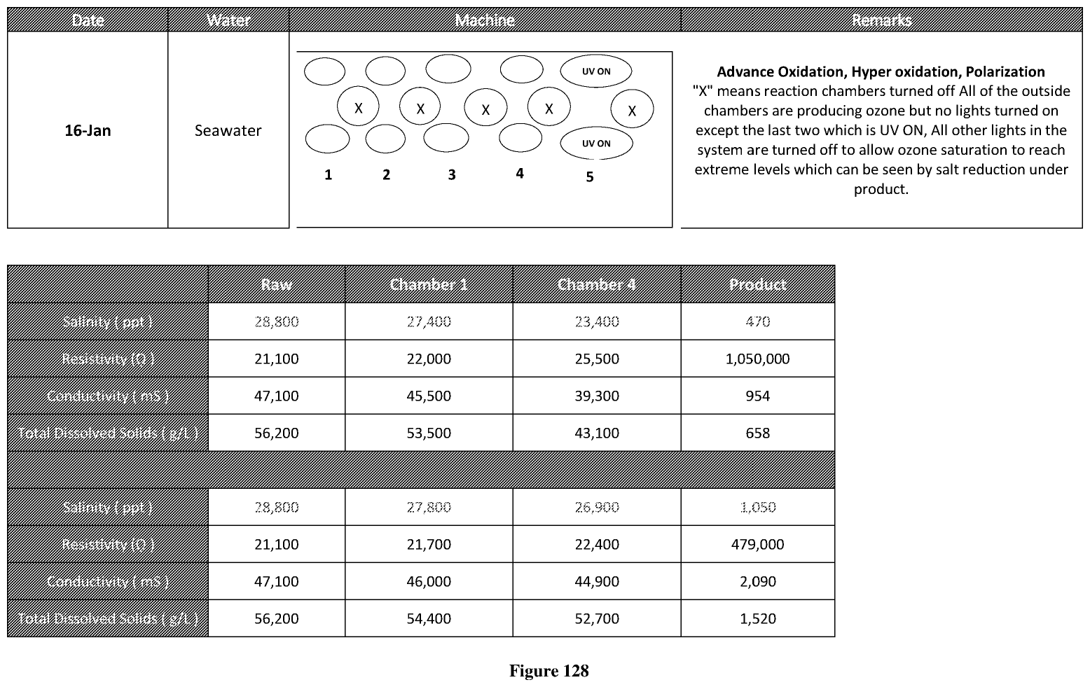

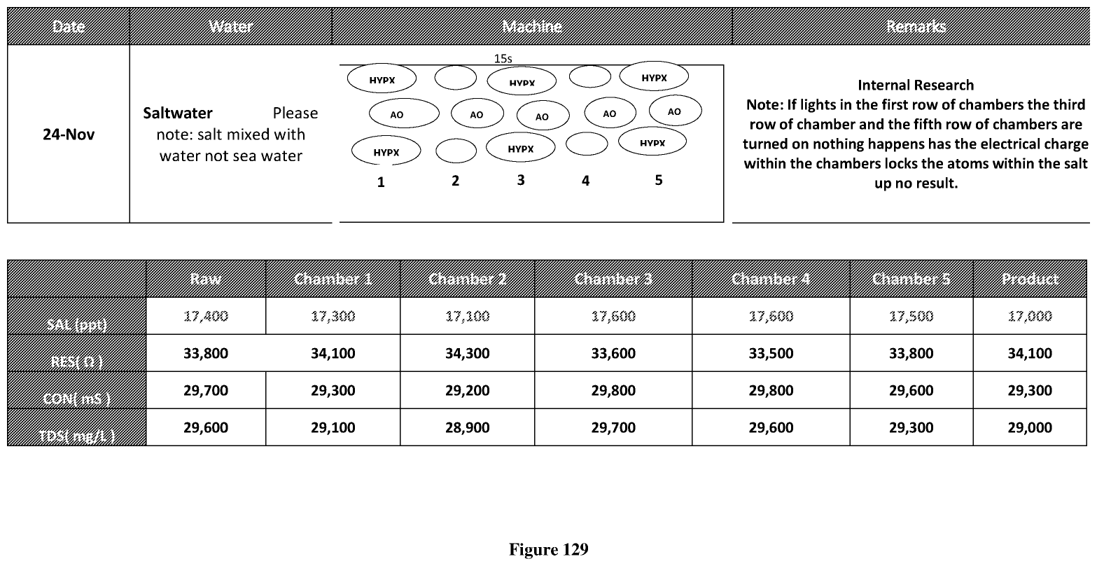

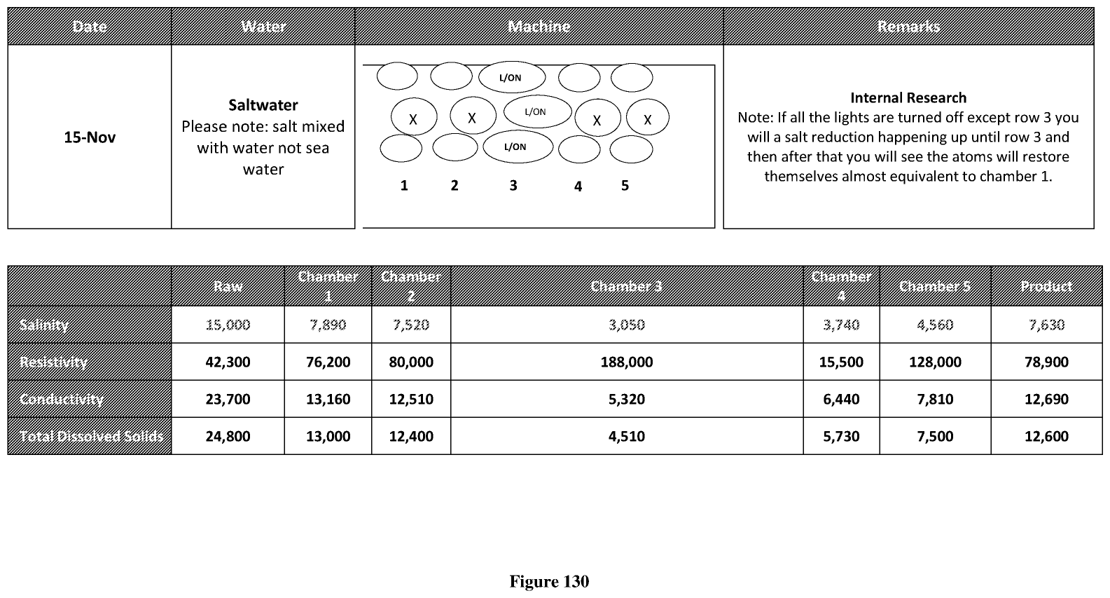

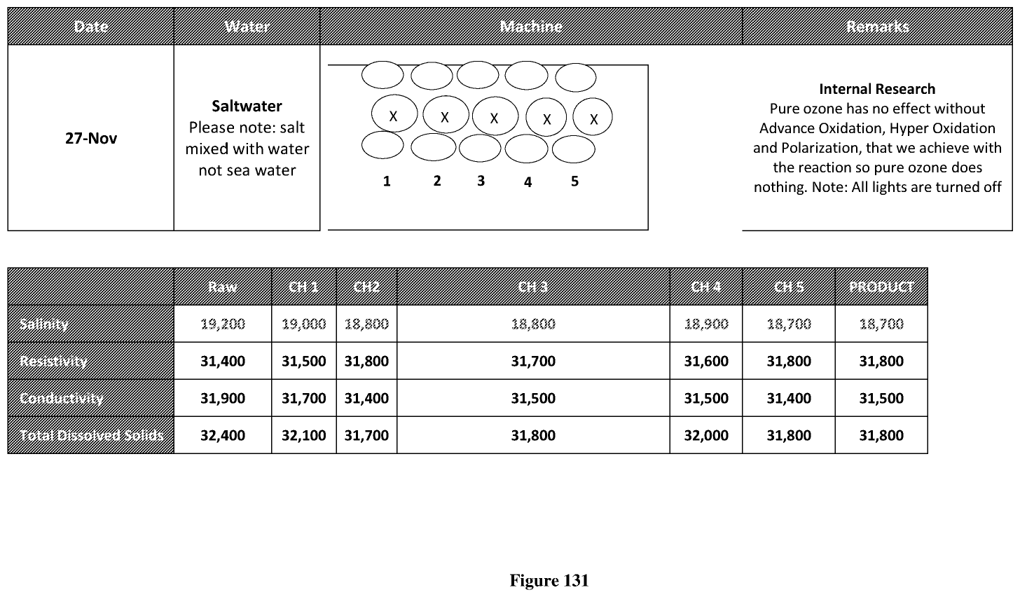

FIGS. 128 to 131 show four different treatment chamber configurations similar to those illustrated in FIG. 109. The four configurations in FIGS. 128 to 131 have been used to treat seawater in FIG. 128 and saltwater (being a mixture of salt with water) in FIGS. 129 to 131. The configuration of the treatment chambers is illustrated in each Figure in the portion labelled “machine” with the fluid to be treated flowing left to right through the chambers (similar to as described for FIG. 109).

The salient characteristics of the fluid which is treated can be seen in the table below the machine configuration measured at the various positions though the respective machine.

Without wishing to be limited by theory, the inventor of the present invention describes operating principles of the invention in the following terms:

“A commonly known fact: if a liquid is placed in static electric fields, the field exerts a force on any free carriers of electric charge in the liquid. Therefore, it conducts electricity. There are two kinds of carriers: conductivities that are generally about one-third of the conductivity of the corresponding solid. The decrease in conductivity upon melting arises from the greater disorder of the positive ions in the liquid and hence their greater ability to scatter electrons. The contribution of the ions is small, less than 5 percent in the liquid metals, but it is the sole cause of conductivity in molten salts and in their aqueous solution. Such conductivities vary widely but are much lower than those of liquid metals.

Non-ionic liquids (those composed of molecules that do not dissociate into ions) have negligible conductivities. But they are polarized by an electric field; that is, the liquid develops positive and negative poles and also a dipole moment (which is the product of the poles strength and the distance between the poles) that is oriented against the field, from which the liquid acquires energy. This polarization has three kinds: Electron, Atomic and Orientation. In Electron polarization, the electron in each atom is displaced from their usual positions, giving each molecule a small dipole moment. The contribution of electron polarization to the dielectric constant of the liquid is numerically equal to the square root of its refractive index. The second effect is the atomic polarization, arises because there is a relative change in the mean positions of the atomic nuclei within the molecules. This generally small effect is observed at radio frequencies but not at optical (can only be seen through specialized ultraviolet lenses particularly through high-quality camera lenses), and so it is missing from the refractive index. The third effect is the Orientation polarization occurs with molecules that have permanent dipole moments. The molecule that is aligned with the field and contributes to the polarization. The above are commonly known to create the difference between a commonly known theory and creating equipment such as polarization that creates the reaction to cause polarization.

Polarization in the system of the present invention is also stimulated because of light waves from ultraviolet and the ozone bubbles which carry very high levels of hydrogen and lower levels of oxygen which rise up through the tubes or vessels, these ozone bubbles are highly energized as they have passed over a 180 nanometer ozone producing ultraviolet light which in volume can produce an electric charge into the bubbles which react with the 253 nanometer lights. These light waves continue to collide and split. Hence, they split into literally thousands of different wavelengths of light creating a guarantee of polarization. As the result of the polarization/sub atomic reaction any product or chemical, which has an atomic or molecular structure, can be broken up into negative zero, according to Einstein's theory, therefore it is still within the fluid or substances. However, it is no longer a chemical because it's no longer a structure. E.g. if clothing dye is pumped through the water dragon the colour turns clear within seconds, the reason for this is that the molecular structure of the chemical which is the dye is broken into negative zero the chemical has no molecular structure, therefore, it is no longer a substance hence the disappearance of the dye. In relation to heavy metals the atomic structure is destroyed or broken up and therefore heavy metals are also removed from the water or fluids, once again negative zero (−0) is created. By increasing, the electromagnetic waves, which are created by the light waves and by turning off some ultraviolet varying reactions, can be created. The hydrogen levels keep increasing. Remember that super high levels of hydrogen keep increasing in each chamber until coming in contact with ultraviolet light waves which then react with the super high levels of hydrogen bubbles ozone to create a polarization/subatomic reaction in water which breaks down atoms and chemicals creating negative zero. If the power level of the 180 nanometer light is increased the level of ozone which means very high levels of hydrogen and lower levels of oxygen and the destruction of nitrogen because the nitrogen atom is easily destroyed, once this is removed the hydrogen will have a much greater reaction within the chamber, and this is the reason why we can also create advanced oxidation without the need for hydrogen peroxide. This reaction is limited by the controlled creation of the hydrogen and oxygen within this environment as they are added to the system. It will create polarization by totally destroying or breaking up atoms and chemicals with a molecular or atomic structure. Polarization has the ability to break down almost all known substances listed above; this is because, within one apparatus, the following effects can be produced. The polarization system produces ‘pure ozone’, (no nitro-acid is produced) which is introduced against the flow of the water in a ‘Hyper oxidation’ polarization reaction chamber. The water then passes into an ozone chamber to enrich the hydrogen levels moving upward with the residual ozone to the top, where the water is divided and the process begins again with pure ozone added every time and this is repeated for as many chambers as there are in the system. Our ozone is produced by the use of of 180 nanometer light waves rather than electrical corona discharge so we do not produce any harmful nitric acid in the process. Additional live ozone must introduce a static electric field, which polarizes the liquid, and all contaminants held in solution are converted into harmless molecules such as water, carbon dioxide, and mineral salts. Bromide and any resultant bromates are destroyed in the same processes. Ozone destruction can be employ at the end of the system depending on the next stage requirements.

The atomic polarization reaction will destroy bacteria, viruses, parasites and residual organic matter that are dealt with early on in the system. The chemicals and heavy metals are particularly susceptible to the polarization processes that also effectively remove hormones and pharmaceuticals. It is a growing problem especially in recycled water, which no one seems to know how to tackle the impending problem.

The preferred system operates at a pressure below 5 psi and employs single or multiple reaction chambers using the following nature-based sciences to treat the polluted water:
Ozonization
Advanced oxidation
Extreme advanced oxidation
Ultra Violet Sterilization
Atomic Polarization
Fractionation
pH Improvement
Negative Zero
Positive Zero

The Polarization Water Purification System Technology:
Provides a quantum leap in capability over current water treatment and purification processes and technologies
Is applicable in all environments and temperatures
Provides chemical-free, healthier, cost-efficient and more effective solutions to drinking water and all polluted water and gas purification and treatment application worldwide
Is modular in construction and where the source water requires further treatment additional reactor compartments can be added to the system.

The Polarization Water Purification System had been designed to:
Disinfect waterborne bacteria
Remove chemicals (polarization destroys chemical molecular structures)
Remove pharmaceutical Compounds (and polarization destroys the atoms and molecular structures
Remove detergents and their residues (and polarization destroys the molecules and molecular structures)
Removes heavy metals (and polarization destroys the atoms and molecular structures)
Removes proteins (and polarization destroys the atoms and molecular structures)
Cyanide & Arsenic
Benzene is a hydrocarbon and is easily broken down (
Eliminate discoloration to produce crystal clear water
Remove hormones from water such 17 bio-estradiol (e2) and the synthetic hormone 17 ethynylestradiol (ee2) these two hormones are usually responsible for estrogenic activity in domestic sewage and are disruptive in severe ways to the male gender. One pass through the Polarization System removes all measurable traces of e2 and ee2 (These hormones are present in contraceptives taken by women). (Almost all pharmaceuticals can 5 be destroyed within the system.

The restriction to using ozone in most cases using cold corona discharge systems the creation of nitric acid (HNO₃) will create blood clotting at even very low levels, so in most cases it is not practical to apply; also, the result of the ozone is 20.9% plus 70% toxic nitric acid, in comparison, making ozone with ultraviolet light of around 180 nanometers created ozone without the nitric acid. But when used in water which has bromide in it the bromide is converted to bromate when added to ozone/ozene causes a very cancerous substance in it, ozone turns bromine into bromate and bromate causes cancer to humans and animals. Bromate when created in the system is immediately destroyed with the breakdown of chemical compounds but bromate is destroyed within the Polarization System.

In remote areas and third world countries, the purification of contaminated water provides further challenges. Available water supplies are often contaminated. Moreover, the water supply is sometimes limited and can be exhausted seasonally, requiring users to move to another supply. Under these conditions, a system for water purification must be robust, simple to use, automated with robust controls mobile and relatively inexpensive. While treated water can be transport to these locations, such transportation is expensive for a heavy bulk item that is used in large quantities. In addition, transportation and storage pose potential recontamination issues.

Accordingly, it would be advantageous for the present invention to provide a convenient, which generates one, or more of the disadvantages set forth above or at least provides an alternative by mounting the polarization system in shipping containers for ease of transport, and also remove the need for buildings, and the like.

Aquaculture system can also be mounted in this way by using two containers with one side removed joined together with the polarization system mounted in a single container with a double container containing fish tank and out of 5 containers 4 can be growing fish and 1 container used for removing ammonia and other toxic substance in center or 1 container processing water. This can be mounted in various configurations and with the ability to have containerized climate control, stackable aquaculture systems requiring less space and better installation from fluctuating temperatures. As long as the water is cool enough to flow, all toxins can be removed and high levels of oxygen that are normally available can be achieved.

In accordance with the one aspect, this invention provides water/gas or a fluid/gas treatment system compromising a single or series of interconnected upright horizontal and vertical or slanting chambers or transfer pipes. The series of chambers has one inlet to the system and one outlet where the inlet is located at the same height as the outlet of the series of a chamber or in equal height in the case gas and or fluids. Gas destruction the inlets and outlets can be at the opposite of fluid treatment by coming in at the bottom and out at the top. The series comprising one first chamber composed of an inlet for fluid to treat at the upper end of the first chamber and the outlet from the first chamber is in the lower end of the first to the outlet. It means for introducing ozone gas in a form containing no nitric acid into a lower end of the first chamber for bubbling upwardly and through the liquid downwardly in a swirling motion through the first chamber and a tube extending horizontally first chamber and housing a first ultraviolet light. This combination will create polarization and when applied at the right density and energy levels. At least one second chamber comprising an inlet for fluid to be treated at the lower end of the second chamber and the outlet of the second chamber at the upper end of the second chamber such fluid flows upwardly or through the side of the second chamber by having the transfer pipe inside of the first chamber starting from the base and moving into the second chamber through the second chamber from the inlet to the outlet. At least one tube extending longitudinally inside of the second chamber and housing a second ultraviolet light wherein the first and second chambers are connecting to the fluid that passes from the first chamber into the second chamber. The ultraviolet lights in the second main chamber will be horizontal and of 253 nanometers or similar plus additional light waves of between 0 nanometers to 1200 nanometers and above. Any other variant of light wave required to give appropriate reactions. Moreover, the outlet of the second chamber that is located below or the same level with the input of the first chamber such that fluid flows gravitationally or pumped through the fluid treatment system. The first chamber may also have horizontal or vertical ultraviolet lights or varying wavelengths.

Preferably, the means for introduction of pure zone bubbles into the lower end of the first chamber can comprise. One ozone generator connected to at least one longitudinally extending ozone chamber, an air blower or pump for blowing air or pure oxygen into a manifold connected to the ozone chamber, an ozone control valve to control the flow of energized ozone gas. In addition, the ozone generator uses O₂ or pure oxygen for an increase oxygen level which is pushed through the ozone in a bubble-creating medium in the lower end of the first chamber. Such as ozonebubbles, it will flow upward through the fluid flowing downwardly in the first chamber. Then, at least one ozone generator may be an ultraviolet lamp generating radiation from the said lamp to a frequency to convert oxygen in said air into ozone. The electrically charge ozone bubbles creating medium may be any one of an air stone or venture unit or similar or into at a lower level which creates the bubbles which bubble against the fluid flows in a downward or into a lower level direction within the first chamber.

Alternatively, an inlet valve may control the control of the rise of the ozone bubbles. Each first chamber may have a clear view window located in an upper section of the first chamber, the window allows the ozone bubbles that can see in the first chamber. This is designed to allow open flow or high enough to prevent overflow.

Preferably, the system may further compromise a mean at the upper end of the first chamber for removing waste in the liquid conveyed by the bubbles upwardly through the chamber. The means at the upper end of the chamber for removing waste may compromise an inverted U-shaped trap or section with bypass valve mounted under it.

A quartz tube can insert into the first chamber horizontally or vertically that may extend across the width of the first chamber or vertically between the horizontal cross sections in the quartz tube protects the ultraviolet light from the inside solutions of all chambers. These lights may also be LED lights.

Preferably, one tube may extend substantially the length of the second chamber and the tube protects the ultraviolet light from the fluid in the second chamber.

Preferably, the tube may be manufactured from clear quartz or from any like materials; the tube allows the light wave from the ultraviolet light to reflect from the ozone bubbles and reflect a react around the chamber by including 180 nanometer lights inside quartz tube additional ozone may be created to the oxygen and hydrogen bubbles.

By changing the bubble size, the atomic polarization reaction can be changed when combined with varying light waves and ozone levels, this creates polarization reaction within the chambers this happens because multiple light waves reflecting around the chambers collide with each other and split and create thousands of new wavelengths of light which combined with the charged ozone/ozene bubbles create a lightning type reaction (polarization).

Preferably, reflective materials may place on the inside of all chambers to improve the reflection of the ultraviolet light in the chamber. The ultraviolet light may have a wavelength of approximately 253 nanometer, any other suitable wavelength, or a mixture of wavelengths even a mixture of normal ultraviolet and LED ultraviolet, but because of the reaction, many new wavelengths are created during the polarization reaction.

Preferably, the system may further comprise an end cap which is releasably attached to the top of each chamber and in which the tube is mounted, the end cap has an aperture through which the ultraviolet lamp is passed into the tube. The second chamber may comprise two tubes or as many as required and in any wattage with ultraviolet lights extending longitudinally each tube.

Preferably, the first ultraviolet light or the means for introducing ozone bubbles may switch between active and non-active status to effectively changing the sterilization process in the second chamber and increase or decrease the electrical and hydrogen energy in the water or fluid as to assist in the creation of negative zero reaction or atom and molecular destruction via polarization.

Preferably, the ozone generation bubbles may use to assist the gravitational or pumped flow of fluid through the exit of the system.

Preferably, the waste removing means of each said first chamber is connected to one or more waste pipes that are more common. When creating negative zero or atomic polarization or molecular breakdown. The lower ends of the said first and second chambers may be selectively connectable to one or more common drainage pipes or ducts via control valves to allow drainage of the first and second chambers along with any excess ozone or any other gas.

Preferably, the first and the second chambers may be located in an in-line system. The fluid treatment system may comprise two first inline chambers and an alternating first and second chamber arranged in a line or is a single or doubled chamber system. Alternatively, the fluid treatment system may comprise a plurality of chambers. The plurality of the chambers may comprise an equal number of the first and second chambers or one single chamber.

Alternatively, the first chambers may be set out in the system in a zigzag pattern and likewise, the second chambers may set out in the system in the zigzag pattern.

Preferably, the system may further comprise a plurality of the fluid treatment system and wherein the inlet from the first chamber is, connect to the outlet of the immediately preceding second chamber. In addition, the lower ends of the first chamber and second chambers are in substantially the same horizontal plane such that the system can be freestanding or supported.

Preferably, the system may further comprise at least one pre-filter located in the inlet line before the first chamber for removing any large materials from the fluid to be treated.

Preferably, the system may further comprise four tubes with ultraviolet light in each tube. The second chambers may manufacture in different diameters to accommodate different numbers of and different sizes of the ultraviolet lights and the tube are mounted.

Preferably, the second chambers may comprise chambers of varying diameter from the top of the chamber to the bottom of the chamber or alternatively have variations where the smaller chamber pipes are located and mounted above large pipes below, which create additional capacity and allow fluids and gases a longer processing time in the system. Thus, allowing for a larger flow of substances to be clean and purified or destroyed by polarization.

Preferably, the system may further comprise a U-shaped bend or trap to retain a level of water of fluid in the waste outlet to prevent any ozone gas leaking into the waste drain and any gases can be re-directed into the gas destruction system to eliminate ozone and turn it back to oxygen.

Preferably, the first and second chamber may be vertical or semi-vertical elongated chambers. The system may be formed as a symmetrical unit with first and second chambers formed of vertically elongated chambers, which decrease in length or height starting at the outer sides of the system and decreasing in height or length until in the middle first chamber is reached, alternatively the system may be of equal height or close to equal height.

Preferably, the first chambers may be operating as extreme advance oxidation delivery units; an ozone gas delivery tubes delivers ozone gas to an ozone spray delivery units located within the first chamber Preferably, the ozone produce either pure ozone or a high level of ozone in the bubbles rising upwards against the water streams and passing ultraviolet lights of 253 nanometers or similar light waves will create polarization capable of breaking down heavy metals, chlorine, chemicals, and organics. The reason this happens is the atomic-molecular level because of the polarization process.

Preferably, ultraviolet lights of different wavelengths may be installed in either first or second chambers and are selected from any one or more UVA, UVB or UVC wavelength lights wavelengths of light or LED light waves covering a much larger nanometer spectrum or variations of different wavelengths in one or more chambers not previously used in treatment of fluids such that the oxidation is possible for substances which have not previously been treated.

Preferably, a combination of different wavelengths lamps may be used in the ozone chambers to produce ozone gas as long as they are close to 253 nanometers

Preferably, any one or more second chambers with fluids without bubbles passing one ultraviolet light of wavelength approximately 253 nanometers or similar can be used in the system to perform advance oxidation of the fluid by working and reacting with high levels of residual ozone.

Preferably, the ozone used in the system may be nitric acid-free however, the used of cold corona discharge may be used as nitric acid will be automatically be destroyed in the polarization process because of the molecular or atomic breakdown caused by polarization.

Preferably, an ozone live bubbles oxidation process may occur in each first chamber or only in selected chambers depending on the reactions required

Preferably, the fluid system may have been designed where valves are fitted to an exhaust outlet of the chambers containing fluid to treat backpressure if required fluids going through the exhaust vent force them to continue into the following chambers.

Preferably, with the present system it may possible to have ozonation, fractionation, chlorination, ozonation, extreme advance oxidation, and ionization, all reacting in the same chamber at the same time due to polarization reaction. Alternatively, any combination of the above as variation to create different reactions whenever is required.

Preferably, by varying the setup or the components within the system, the pH level may raise or lower as required. (See lab report fig no. 89) Because of oxidation concentration or if pH is too high it will be reduced.

Fractionated fluids or gases may be removed at the upper end of the chamber through an exhaust or waste pipe which may have a controlling valve which may be turned off, left, or partially open so as to create back pressures for said gases or fluids which will be required in normal operation. The backpressure created will contain the fluids.

These fluids may be sterilized using ozone and multiple wavelengths of ultraviolet light if required. These ozone bubbles rise to meet wavelength of UV such as 253 nanometers or similar light waves to create atomic polarization. When viewed through ultraviolet lenses, the effect is like looking at a thousand lightning strikes per second and almost appears that the chambers are on fire without producing any external heat. The UV bulbs may be mounted to the top part or lower down in expanded chambers of the embodiment. They may also be horizontal and or vertical or both.

The effects of the extreme atomic polarization are to destroy the molecules of the chemicals and in an atomic way to destroy heavy metals and in some cases, oxidize certain metals in a very efficient way. Some metals such as copper is removed at the upper exhaust tube or waste by ozone fractionation with the use of ozone bubbles before meeting the polarization process or air that is most preferable for the particular water or fluid and becoming a partial solid at the base of the collection chamber because of the molecular change in the change of water molecules. Other metals such as Zinc, Chromium, Nickel, and many other metals may also be recovered or removed

As multiple chambers may use a standard embodiment will need to be mounted on a level surface however embodiments such as the zigzag are designed so as to be mounted in such a way that every rolling motion such as at sea will not affect the embodiments function. In a zigzag format, the fluid or gases must pass through each chamber. Atomic polarization can be created in a chamber the ultraviolet lights may be turned on and off at will to change the reaction process.

All of the above reactions in the different embodiments have major destructive powers on viruses, bacteria, moulds, spores, protozoa, cyst, fungal pathogens, algae, fungus spores, and moulds spores, yeast. The removal of the biological oxygen demand (BOD) and the chemical oxygen demand (COD), natural or synthetic hormones.

In relation to chemical atomic molecular destruction, poisons such as cyanide and arsenic can easily destroy. The destruction of chemical molecules using polarization has a devastating effect on almost any chemical or poison.

Radioactive water or heavy water from a nuclear power station or nuclear facilities that can be treated with varying forms of these embodiments. The destructions of the metals of the radioactive particles within the water such as uranium caesium (Cs) and plutonium, which are primary metals of varying types, should be destroyed within these embodiments by varying the settings to suits the purpose. Heavy water should turn to light water with vastly lower levels or radiation. When using atomic polarization using various wattages and ozone levels.

The embodiment variations that be created by changing the settings and with the inclusion of ozone fractionation combined with ozonation, chlorination, advance oxidation creating polarization has the ability to breakdown the elements into chlorine gas and hydrogen sulphide which is extracted through the exhaust or waste discharge pipe for collection and reuse as fuel. Other elements are oxidized and/or molecularly dissipated thus leaving fresh germ-free water in any portion depending on the size and structure of the embodiment and also, providing fresh drinking water. No sodium as a physical form is produced in this process.

The combination of ozone fractionation and extreme advance oxidation, chlorination and ionization along with varying ultraviolet wavelengths can combine in one embodiment to produce atomic polarization. The ability to switch between active and non-active can change the reaction within the embodiment at the turn of the switch. The gases created may be dried, compressed to a suitable pressure, for burning, for the production of the steam or any other requirement as required.

Any gases produced or as a by-product of the burning gases that can be redirected through the gas destruction embodiment with or without water to neutralize any toxic or any pollution in the exhaust system.

Within the embodiment, carbon dioxide can be passed into chambers containing pure ozone then passed into 253 nanometers ultraviolet light to be broken back to oxygen (O₂) and then into ozone (O₃) and back into multiple chambers of ultraviolet at 253 nanometers, or both ozone and ultraviolet combined in one chamber. This process may produce oxygen (O₂) capable of being recycled or reused for other purposes or continues use of air from carbon dioxide or carbon monoxide to oxygen and Polarization may be possible.

Within this system by using the varying wavelengths of lights, carbon dioxide can be converted back to oxygen for to improve the level of oxygen by creating ozone and then with the use of different wavelengths of ultraviolet light break the ozone (O₃) back to (O₂) or oxygen.

Because of the polarization reaction between the ozone bubbles and the UV light waves bouncing from the bubbles which can act mirrors or prisms and because light waves are refracting and bouncing around the chamber, the light waves also collide reacting and creating an electrical field of literally thousands of wavelengths which have been created from one wavelength. This electrical charge within the reaction chamber could be utilized as a power source.

The system of the present invention may include multiple treatment chambers of the same or different configurations, in line or side by side with angled connection pipes, vertical-horizontal pipes, horizontal and vertical pipes and horizontal. All of these connection pipes preferably have a coupling in top and bottom and may be removeable for cleaning outside of the housing chamber, or such pipes can be mounted internally to move water from the bottom to the top or top to the bottom internally or to another chamber.

The differing connection pipes allow for variation in operation and can carry ultraviolet bulbs of various wavelengths of light waves, to create varying reactions in the water or gases.

By changing the connection pipes to the various designs such as ozonation, fractionation, advanced oxidation, extreme advanced oxidation and polarization can be created simply by coupling and uncoupling different configurations as shown in the Figures. All of these reactions can be created without the use of hydrogen peroxide as a catalyst because of the high levels of hydrogen created from the extremely high levels of ozonation because no nitric acid is captured or created within the system. Therefore, the present invention can achieve use of a hundred times the ozonation levels of any other system. These connection pipes may have ozone or air bubbles included to create additional reactions or assist with a pumping action utilizing the rising bubbles.

The main embodiments of treatment chambers can have fluid or gas transfer chambers fitted to one side or both sides or can be installed internally and alternating sides as singular, doubles, triples or in any number to carry the required flow. Also, may be directly transferred internally through the side walls of the next chamber to reduce flow time and the room required to install such system.

The main embodiments of treatment chambers may be of equal height, slightly higher or lower than the main embodiments next to them and can placed in single rows, multiple rows or in any form that allows water or gases to flow between the treatment chambers.

The connection pipes can be different from the main embodiment chamber in many different formats to achieve the result required as shown from FIG. 1 to FIG. 9.

The system can be used for fluids such as polluted water caused from chemical contamination, heavy metal contamination, viral bacteria contamination, and radiation contamination or colourings such as clothing dyes and other colouring agents. The destruction of these contaminants can be created by destroying or disrupting the chemical molecular structure or the atomic structure of various heavy metals. The biological structure of viruses and bacteria are destroyed in the following ways, extreme levels of pure zone containing no nitric acid these levels can be hundreds of times higher than can normally be achieved.

The invention can be change from one reaction to another simply by turning of lights whether it be in transfer chambers, fractionation chambers that can be in the transfer chambers or within the main embodiment. The amount of different reaction can be created in one system would be in the hundreds depending on the size of the system.

These systems can treat fluids or gases and air in one single system for example the front six chambers may be treating water and remaining four chambers may be treating gases or air or destroying ozone by turning it back to purified air.

By placing the varying ultraviolet bulbs in rows inside quartz housings, the water or fluids can be rotated around the system and slowly work the way down through the system pushing against ozone bubbles rising or air bubbles as they pass over the varying light waves, the bubbles have the effect of reflecting the light by acting as mirrors or prisms. When the light waves reflect around the chamber they have the effect of colliding with each other splitting and creating new light waves, which are in the thousand, the effect of these reactions create polarization and can only be seen through specialized UV lenses. Also, by adding 180 nanometer tubes along with 253 nanometer tubes and other wavelengths as required. As oxygen or air passes the 180 nanometer tube the oxygen in the water will convert into ozone.

By changing the wavelengths of different bulbs and placing them in different places throughout the chambers many different types of polarization can be created. The number of differing reactions is only limited by the imagination wavelengths from 10 nanometers to 1200 nanometers and above, such as that which can be created by LED lights. There is preferably little or no heat created within the system. As different wavelengths are created and mixed together when viewed through a specialized UV lens different colors of light waves can be viewed flashing around the chamber.

Electrostatic charge is created in two ways by pumping air over 180 nanometer ultraviolet lights and electrostatic charge is created within the ozone bubbles which form in the multiple chamber. In addition, the bubbles and water passing over ultraviolet lights of 253 nanometers or any other wavelengths also create an electrical discharge. This discharge must be controlled as excessive electrical discharge can have the effect of turning negative zero which chemical molecules and heavy metal atoms are broken down within the system and create the negative zero holding the various elements apart.

Excessive energy can be withdrawn from the system by using the chlorination system within the chambers as negative to earth. This will control excessive overcharging of the system if required to stop any molecular lock up within the system.

The excessive electrical charge can be reduced with the use of air bubbles passing through the water without lights, even with lights the amount of charge in the water or fluid is reduced. These electrical charges in the system must be controlled, in other ways, to reduce electrical charge is by speeding up the flow of the fluids or gases so they actually spend less time within the system and therefore hold less charge.

The system of the present invention has been designed to reduce salt from water or simply to reduce salt to required levels because the system has the ability to knock out minerals and minerals are metals and metals are atoms, the aim on treating the salt water is to knock out a lot of the 47 minerals and metals in the 40 water. This has the effect of reducing the total dissolve solids to an acceptable level, thus reducing the salinity of the water's dissolve solids to an acceptable level, thus reducing the salinity of the water.

By allowing the ozone level to increase to such high levels the effect in the following chambers 45 when passing through varying nanometers of ultraviolet and the polarization that the salinity of the water is dramatically reduced this can be adjusted from chamber to chamber. The advance oxidation and the extreme advance oxidation along with the polarization reducing the total dissolved solids (TDS) if the TDS is very low then the salinity is very low all of this can be done without filtering.

The system is designed to be fitted with chlorinators so in one chamber ozonation, chlorination can all be applied inside the chlorinator. Chlorine gas can be created as part of the process this has the effect of reducing the salinity levels and creating a usable fuel for the production of steam to drive turbines and produce electricity, the by-product of burning chlorine is carbon monoxide any by-products can be destroyed by passing the exhaust gasses through the system in the reversed manner to which it normally runs in other words, from the bottom to the top and by closing off any exhaust outlets within the system.

Another effect of the system is to spray water into a stack or chimney this will have the effect of taking the toxins from burning coal such as lead, mercury, CO₂, as well as many other destructive toxins and placing it into the water along with any ash this will have the effect of reducing the pollution from these stacks up to 90% making coal and other fossil fuels a green energy source. Once the toxins are in the water they can be pass through the water dragon system and destroyed, this means no ponds required for water storage instead it can go straight back to the stacks again on a repeating cycle with only make up water required for the evaporation.

In addition to chemicals, heavy metals and biological issues the water dragon system with its multiple powerful combinations of reactions can destroy pharmaceuticals of any type including hormones which mainly distributed by the contraceptive pill and as with all pharmaceutical, which are synthetic, so they do not break down over time consumed by the human population they will all pass through our urinary track system in a form of urine and finishes up in 20 sewage plants and septic systems which also soak into the underground water system and eventually pump back to the population. This has the effect of creating massive side effects on humanity.

When passing toxic gases through the system, the system preferably has the ability to destroy many if not all of the toxins within the gas stream by adding air or oxygen to the system along with the gas and by placing exposed 180 nanometers ultraviolet bulbs in layers above or below 253 nanometers bulbs, the ozone will be created right next to the 253 nanometers ultraviolet these reactions will be enormous and instantaneous additionally by placing 180 nanometers and 253 nanometers side by side in multiple layers within each chamber along with various other bulbs of vary nanometers total destruction of most gases will occur.

By passing the gases through venturi or air stones, the gases will become trapped within the water. These compounds can then be destroyed by the advance oxidation, extreme advance oxidation and polarization. This provides to varying ways to destroy heavy metals, chemicals and toxic substances.

The waste pipe carrying any fractionated water bubbles along with any dirt or discoloration will be partially vacuumed out of the system by placing a 45-degree angled outlet pipes to the waste drain and being pumped into one end of the waste pipe will create a vacuum and instantly start the breaks down of any residual ozone into clean fresh air. At the discharge end of the waste drain, a U-bend will 40 force the gases and the water or fluids to separate the gases which can then be treated to remove any residual ozone and only emit clean air.

Ozone and ultraviolet tubes can be left exposed within the chambers both horizontally and vertically when using gases. However, in the case of fluids, the light must be housed in quartz tube to keep them dry; these tubes can be also in horizontal, vertical or in any practical format.

When placing gases through the system the first 5 chambers can be kept dry purely as a gas destruction at the end of these process any gases left can be placed into the water by venturis under pressure and the second half of the system can be a liquid based system applying additional destruction applications.

Within the system, negative zero energy is created this has the effect of splitting the molecules and dividing the molecular structure split each portion of the molecule and turns then into negative zero in this form, the chemical no longer exists and because of its portion of molecular structure is now negative zero, it cannot be reformed, However in sea water the test results establish that the system can reduce the sodium and the total dissolve solids in the case of chemicals such as dioxins (Dieldrin for example) when passed through the system become not detectable and in these test no waste discharge occurred. Adjusting the number of lights and/or their position shows differing reactions.

Chemicals, heavy metals and pharmaceuticals when pass through the system are destroyed in one or more of molecular and atomic breakdown; many of these contaminants are turned into negative zero, carbon dioxide and some salts when treating sea water the destruction of removal total dissolved solids (TDS) is the answer to the salt from the system by destroying the minerals in sea water which means the TDS will drop to 35,000 to less than 250.

The system has the ability to produce ozone from air when the 180 nm ozone producing bulb are place in the system with ozone, air and pure oxygen are passed over the bulbs within the system the expose ozone bulb will create ozone (no nitric acid is produced) are submerse inside quartz tube in the water in varying position across the chamber and vertically the oxygen in the bubbles passing the ozone producing tubes will produce ozone on contact or when penetrated by light waves produce within the lights.

The system when fitted with varying light waves in layers or side-by-side of 180 nm and 253 nm can create enormous destruction to living organisms, heavy metals, chemicals and pharmaceuticals along with unknown toxic gases. This has the effect of cleaning substances and destroying gases previously thought to be untreatable.

By exposing bulbs directly in the water but sealed from the water from each end by quartz tubes or similar and by varying the wavelengths with the used of LED lights and from its lowest of 0 nanometers to late 900 nanometers and above to the thousands and by placing various wavelength in close proximity in the chamber to each other destructive powers that can clean, destroy, oxidize and to reform some substances will be possible. The amount of electrical charge must be controlled so as create negative zero and not allow positive zero to reform so various systems such as earth chlorinators or wiring harness which can control the static electrical build up of the water or fluid or gases.

Gas destruction can be most efficient and by passing through 1, 2 or 20 chambers of various settings of nanometers and by using low and high energy bulbs and by flowing the gases adding air or oxygen to increase reaction either by adding it from the top, sides or base and by adding ozone, air and oxygen in various ways gas destruction, germ destruction is assured. The speed of flow of any gases added to the system must through the control rate to eliminate unwanted gases at the end of the process. Electrical charge within the gases must be monitored and reduce where required.

It Is in relation to a floating version of this invention a setting of the ultraviolet lights and the positioning of the ultraviolet lights will create advance oxidation and extreme advance oxidation and polarization when position above ozone bubbles diffuses or micro bubblers.

This portion of this invention allows the invention to become mobile in rivers, lakes, dams or oceans by lowering the platform and propelling the vessel through the water with the platform in the lowered position with the lights and ozone bubblers all working the water driven through the platform will create the treatment reaction. The speed in which the vessel is driven through the water will control the reaction and the severity of the reaction relating to the water flow and the reaction and time for the reaction of the ozone ultraviolet light exposure.

These floating systems can also be anchored and fix position and have the water driven through the submerged platform by the paddle wheel or pumps and be forced over the platform at any speed which is the correct flow for water to be treated.

Ozone will be produced inside the containers using air over ultraviolet bulbs of 180 nanometers or similar which is then forced into multiple rows of air stones or venturis or micro bubblers to create extremely high levels of ozone without nitric acid being produced and by changing how far apart the ultraviolet bulbs of 253 nanometers and other wavelengths and by changing these positions we can create the same effects as if the bulbs wherein tubes or containers as the level of ozone and the amount of saturation of ozone when meeting the UV bulbs can create the effects of advance oxidation (without hydrogen peroxide) being required to produce this reaction a massively high levels of ozone in the water (hydrogen) combined with air (oxygen) will create the advanced oxidation, extreme advance oxidation will also occur along with differing forms of atomic polarization.

The lifting of the platform allows the vessel to move freely through the water without drag and to clean the platform and service it above water. The double platform which is joined in the middle can be removed and separated and each fit in to a shipping container for transport the bow and stern ends can each be removed and also fit inside shipping containers to transport.

These floating systems can process huge quantities of water hundreds of thousands of cubic meters of water can be processed in this way every day. These systems can be driven through the water or placed in a stationary position or have the water driven through the system by the paddle wheel or pumps or allow the current to flow through the system and control the speed of flow by using paddle wheel to restrict or increase the flow. The same system can be built on land and by sealing. The ends of the platform water can be pump into the system at a suitable speed similar to float in on a river or a lake. The system in this format can be stacked one on top of the other or joined from end to end to create a much more powerful system by allowing the water to have more treatment time before being discharge through the system.

The paddle wheel, which can drive the system or boat through the water, can also be used when the boat is in an anchored position or when floating freely in the water to drive water over the plate form from the back or pull the water through from the front. The paddle wheel is driven by chain and sprocket or by belts which are attached to a variable speed electric motor with gear box to drive the paddle wheel at the appropriate speed required generators are also placed in the floating containers to provide the electricity for water processing or to move the vessel from place to place to create electricity for the air pumps and ultraviolet lights.

The platform can be used as a biological filter by spraying water from the top and bringing air bubbles from the bottom to oxygenate the water and by adding biological media for the bacteria to attach to remove or convert such elements such as ammonia to nitrate while passing thru the system. The system may be partly submerged to allow the water to trickle over the biological media which will allow the water to become highly oxygenated and remove carbon dioxide (CO₂) from the system by slightly submerging the platform it will force the oxygen coming from air stones to pass upwards through the system and not just dissipate from the system, also by submerging the platform fully and spraying the water from the top these same effects will occur. The system may also be fully submerged allowing the bubbles to rise from the bottom and using the paddle to pull or push water through the biological media.

By pumping water through the system form end to end with the air stones operating fully and by applying UV at the end of the system or the discharge in biological reaction will occur with the purification at the end of the system, however by leaving the UV lights off the bacteria which are constantly separating can flow with the water out of the system into lake, river or dam and work within that water because water is now fully oxygenated and now the various forms of the bacteria can continue to remove the ammonia converted into Nitrate and by removing the ammonia the food source of the algae blooms will disappear and therefore the algae will also be gone.

The floating or land-based system can remove salt and minerals by breaking down the total dissolved solids. This can be done by setting the 253 nanometer ultraviolet lights in the center of the system and just prior to the end of the system or no center system just the concentration of light taking the end of the flow process. This can be done on land or in the water. The reactions of the advanced oxidation extreme advanced oxidation and polarization to remove the metals, chlorides and salinity or reduce it to a point where it is drinking water standard. This open system will produce the same reactions as the system shown previously in earlier phases of this patent application.

This molded embodiment allows for fix or removable flow panels these panels can be under and over or over and under or have open ended sections which can flow from one side to the other side create varying reactions in the same way as the under and over. These sections by being removable can allow larger or smaller compounds, which can allow the ozonation saturation to be increased and then create larger reactions of advance oxidation, extreme advance oxidation and polarization. A lid or a top will be fitted to each chamber. The adjustable fractionation waste pipes can be raised or lowered depending on the amount of froth or foam, which can be removed from the system without allowing too much fluid to flow out of the system. These pipes will have a vacuum created by air venturis to assist in a sucking action to create a vacuum to remove ozone gasses from the chambers.

By containerizing the equipment of the system or building it into a multi-story building, the processed water can be made to fall from the various heights and spinning a turbine creating electricity and water storage.

Adding flexible hanging rubber strips as part of any treatment chamber allows separation of the lighting from one chamber to another.

In the new format of the system with crisscrossed UV lights 253 nm bulbs, a single chamber on its own can be used by taking water or fluid in and bringing it up in to an adequate height for discharge will maintain the water level in the chamber.

This new version of the system or single polarization reaction chambers, may also be used as a tank as well as a processing system which adds another element to its productivity. Two single chambers with horizontal UV bulbs or horizontal in vertical UV bulbs may be joined the horizontal transfer pipe with an inlet on a first chamber and an outlet on the second reaction chamber with high wattage bulbs and high levels of ozone with little or no nitric acid with fine bubbles preferably produce polarization.

In one preferred form, waste typically enters the system through angled rotating drum filters where fluids and solids are separated. This solid enters an enclosed auger transfer system and are deposited into a paddle steam dryer where the solids are dried with heat and disinfected with additional injected of ozone. When coming out of the dryer all waste goes over a vibrating screen set on an angle any paper or waste products are discharged by the screen all dried sewage solids vibrate through the screen onto the conveyer belt to be made into compost fertilizer. All the liquid waste goes into two standard filters and is then pump into the water dragon system through the effects of polarization treatment. The result is clean water fit for drinking.

The system can be created as a C-section shaped, round shaped, hexagonal or any shape that will create polarization.

As an added filtering system for the system, a table type belt membrane may be used to eliminate salts and solid from any water source. The continuously moving belt is locate as close as 1 mm to the high-pressure water discharge, which after passing through the membrane belt the filtered water enters chamber and is discharged by pipe. The continuous moving belt carries away salts and debris and goes over the large drums. As it goes over the end drum wheel and passes under the system. The salt and waste derby are now at the bottom of the belt and is remove by a high-pressure air or water for discharge the belt then returns to the high-pressure water inlet for removal of contaminants. This continuously moving membrane belt replaces reverse osmosis membranes, never stops to backwash, and never requires chemical treatment as this is an advanced form of salt or sodium removal.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

### DRAWING PART NUMBERS

PART NO. 1 MAIN EMBODIMENT OF REACTOR HOUSING FOR OZONATION SATURATION
PART NO. 2 MAIN EMBODIMENT REACTOR CHAMBERS, WHICH CREATES OZONATION, EXTREME ADVANCE OXIDATION, POLARIZATION AND FRACTIONATION ALL IN ONE EMBODIMENT.
PART NO. 3 OUTLET OF MAIN BODY REACTOR HOUSING
PART. NO 4 INLET OF MAIN EMBODIMENT REACTOR HOUSING AT THE TOP
PART NO. 5 COUPLING JOINER FOR ADVANCED OXIDATION TRANSFER CHAMBER
PART NO. 6 REMOVABLE ADVANCED OXIDATION TRANSFER CHAMBER
PART NO. 7 REMOVABLE ADVANCE HORIZONTAL-VERTICAL OXIDATION CHLORINATION TRANSFER CHAMBER
PART NO. 8 REMOVABLE EXTREME ADVANCED OXIDATION TRANSFER CHAMBER
PART NO. 9 REMOVABLE OZONATION EXTREME ADVANCE OXIDATION TRANSFER CHAMBER
PART NO. 10 REMOVABLE OZONATION OXIDATION FRACTIONATION DISCHARGE CHAMBER
PART NO. 11 REMOVABLE OZONATION OXIDATION FRACTIONATION RECIRCULATION CHAMBER
PART NO. 12 REMOVABLE OZONATION DISCHARGE HYPER OXIDATION FRACTIONATION CHAMBER
PART NO. 13 REMOVABLE AIR FRACTIONATION CHAMBER
PART NO. 14 REMOVABLE CAP ON TRANSFER CHAMBERS
PART NO. 15 JOINING COUPLINGS FOR REMOVING
PART NO. 16 EXPANSION COUPLING HOLDING QUARTZ TUBE OR UV BULB
PART NO. 17 ELBOW JOINING FITTING
PART NO. 18 SECTION JOINING FITTING
PART NO. 19 WASTES OF DISCHARGE PIPES
PART NO. 20 AIR INLETS
PART NO. 21 ELECTRICAL WIRES
PART NO. 22 HOLLOW QUARTZ TUBE
PART NO. 23 ULTRA VIOLET BULBS
PART NO. 24 DRAIN VALVE
PART NO. 25 DRAIN PIPE
PART NO. 26 CLEAR VIEW SCREW IN
PART NO. 27 CLEAR VIEW PIPE
PART NO. 28 V SECTION FOR WIRING
PART NO. 29 CAP FOR V SECTION
PART NO. 30 FRACTIONATION DRAINAGE
PART NO. 31 REVERSE U SECTION TO SEPARATE THE WATER AND GASES SO THAT EACH PROCESS CAN BE TREATED SEPARATELY
PART NO. 32 EXCESS OZONE TO BE TREATED VIA GAS DESTRUCTION
PART NO. 33 REMOVABLE CAP OR LID OF MAIN BODY REACTOR HOUSING
PART NO. 34 ULTRA VIOLET HOUSED IN QUARTS TUBE TO BE WATERPROOF
PART NO. 35 AIRSTONES, DEFUSES OR BUBBLERS OF ANY PRACTICAL SIZE LIKE FINE, MEDIUM OR LARGE
PART NO. 36 RECIRCULATING PIPE WITH VENTURI
PART NO. 37 CAP OR LID SEALED
PART NO. 38 SECTION JOINERS
PART NO. 39 OVER FLOW
PART NO. 40 ALTERNATING HORIZONTAL-VERTICAL TRANSFER REACTION CHAMBER
PART NO. 41 VERTICAL QUARTS TUBES WITH ULTRA VIOLET BULBS INSIDE
PART NO. 42 HORIZONTAL QUARTS TUBES WITH ULTRA VIOLET BULB INSIDE
PART NO. 43 CURVED HORIZONTAL VERTICAL TRANSFER CHAMBER
PART NO. 44 ANGLE TRANSFER CHAMBER
PART NO. 45 ADVANCE OXIDATION CHAMBER
PART NO. 46 VERTICAL HORIZONTAL TRANSFER PIPE WITH DOUBLE ADVANCE OXIDATION
PART NO. 47 VERTICAL HORIZONTAL TRANSFER PIPE WITH DOUBLE ADVANCE OXIDATION ALTERNATE SIDE
PART NO. 48 ANGLE VENTURI PIPE FRACTIONATION AND GAS DRAIN
PART NO. 49 PURE OZONE CHAMBER
PART NO. 50 VERTICAL AND HORIZONTAL OZONES
PART NO. 51 VERTICAL QUARTZ TUBE WITH UV BULBS
PART NO. 52 HORIZONTAL QUARTZ TUBE WITH UV BULBS
PART NO. 53 CRISS CROSS UV BULBS
PART NO. 54 COMPRESSIONS COUPLING TO STOP LEAKING
PART NO. 55 OZONE INLETS
PART NO. 56 HOLD TO FIT TRANSFER PIPES
PART NO. 57 EXPOSE UV BULBS NO QUARTZ TUBE FOR GAS OR AIR
PART NO. 58 EXTREME ADVANCE OXIDATION AND POLARIZATION
PART NO. 59 OZONE DISTRIBUTION VENTURI OR AIRSTONE
PART NO. 60 S OR U BEND
PART NO. 61 REVERSE U BEND WITH VALVE
PART NO. 62 OUTLET OF GAS DESTRUCTOR
PART NO. 63 INLET OF GAS DESTRUCTOR
PART NO. 64 POSITIVE AND NEGATIVE CHLORINATOR HOUSE IN TRANSFER CHAMBER
PART NO. 65 NEGATIVE CHLORINATOR CAN ACT AS EARTH CONNECTION
PART NO. 66 POSITIVE CHLORINATOR
PART NO. 67 CHLORINATOR
PART NO. 68 40 FOOT SHIPPING CONTAINER
PART NO. 69 ISOMETRIC VIEW OF THE SHIPPING CONTAINER
PART NO. 70 10 FOOT CONTAINER
PART NO. 71 INCOMING FILTER
PART NO. 72 OUTGOING FILTER
PART NO. 73 FILTER BACKWASH DRAIN
PART NO. 74 FILTER SCREEN
PART NO. 75 DIVING WALL TO ALLOW THE FILTER TO BE SEPARATED DOWN IN THE CENTER
PART NO. 76 AIR OR GAS INLET
PART NO. 77 AIRS OR OXYGEN INLET
PART NO. 78 PURIFIED AIR OR DESTROYED GASES OUTLET
PART NO. 79 EXPOSE UV BULBS OF 180 NANOMETERS
PART NO. 80 UV BULBS WITH 253 NANOMETERS
PART NO. 81 PURE OXYGEN
PART NO. 82 AIR AND GASES PURIFICATION CHAMBER
PART NO. 83 AIR AND GASES PURIFICATION CHAMBER
PART NO. 84 AIRS AND GASES PURIFICATION CHAMBER
PART NO. 85 AIRS AND GASES PURIFICATION CHAMBER
PART NO. 86 AIR AND GASES PURIFICATION CHAMBER
PART NO. 87 WATERPROOF OZONE TUBE FOR DIRECT CONTACT WITH WATER, AIR OR OXYGEN WITH OR WITHOUT QUARTZ COVERING
PART NO. 88 CONTAIN IN THE CHAMBER MAINLY OF 253 NANOMETER WITH OZONE BUBBLES RISING AIR OR OXYGEN
PART NO. 89 CONTAIN WITHIN CHAMBER MULTIPLE NANOMETERS OF DIFFERENT WAVELENGTHS IN EACH BULB
PART NO. 90 ANGLE TRANSFER PIPE
PART NO. 91 OZONE BUBBLERS
PART NO. 92 AIR BUBBLES
PART NO. 93 PURE OXYGEN BUBBLES
PART NO. 94 GASES ONLY
PART NO. 95 FRACTIONATION DRAINAGE CHAMBERS
PART NO. 96 SEA WATER OR SALT WATER
PART NO. 97 SEA WATER ENTERING THE WATERDRAGON SYSTEM
PART NO. 98 FRESH WATER COMING FROM THE WATERDRAGON SYSTEM AFTER THE BREAKDOWN OF TOTAL DISSOLVE SOLIDS PRODUCING FRESH CLEAN WATER
PART NO. 99 CHLORINE SYSTEM BEING PRODUCE FROM THE SYSTEM WITH THE AID OF SALT WATER CHLORINATORS WHICH ALLOCATED IN THE TRANSFER CHAMBERS TO PRODUCE CHLORINE GAS FOR BURNING WITHIN THE BOILER TO MAKE STEAM
PART NO. 100 GASES ENTERING THE BOILER TO BE BURNED AS FUEL
PART NO. 101 BURNED GASES FROM THE BOILER ENTERING THE GAS DESTRUCTION SYSTEM
PART NO. 102 GAS DESTRUCTION SYSTEMS
PART NO. 103 CLEAN AIR COMING FROM THE OUTLET FROM THE GAS DESTRUCTION SYSTEM
PART NO. 104 STEAMS FROM THE FRESH WATER ENTERING THE BOILER TO SPIN STEAM TURBINE WHICH TURN THE ELECTRICAL GENERATOR
PART NO. 105 STEAM TURBINE
PART NO. 106 ELECTRICAL GENERATORS
PART NO. 107 ELECTRICITY ENTERING ELECTRICITY GRID
PART NO. 108 SOME OF THE ELECTRICITY PRODUCE GOING BACK TO GAS DESTRUCTION SYSTEM
PART NO. 109 WATERDRAGON DESALINATION SYSTEMS AND GAS PRODUCTION SYSTEM BEING RUN ON FREE ELECTRICITY FROM THE FRESH WATER STEAM AND CHLORINE FUEL PRODUCE
PART NO. 110 COAL OR FOSSIL FUEL FIRED BOILER FOR PRODUCING STEAM TO MAKE ELECTRICITY AND ALSO PRODUCING ASH TOXIC HEAVY METALS SUCH AS LEAD, MERCURY AND MANY OTHERS INCLUDING CO₂
PART NO. 111 ILLUSTRATES A LARGE STACK OR CHIMNEY WHICH NORMALLY WOULD DISCHARGE LARGE VOLUMES OF HEAVY METALS AND POLLUTANTS TO THE ATMOSPHERE
PART NO. 112 SHOWS FALL OUT FROM THE STACK OF ASH AND HEAVY METALS WHICH ARE TAKING OUT OF WATERS AND ACCUMULATE AT THE BASE OF THE STACK
PART NO. 113 SHOWS SLUDGE PUMP WHICH PUMPS THE ASH, WATER, HEAVY METALS AND CO₂ WHICH HAVE BEEN ABSORB BY THE WATER
PART NO. 114 SHOWS SETTLEMENT CHAMBER WHERE THE SLUDGE SETTLE FROM THE BOTTOM
PART NO. 115 SHOWS A FILTER WHICH REMOVES ANY REMAINING SLUDGE WHICH IT RETURN TO THE SLUDGE SETTLEMENT CHAMBERS
PART NO. 116 SHOWS THE POLLUTED WATER ENTERING THE WATERDRAGON PROCESSING PLANT WHICH WILL DESTROY HEAVY METALS AND CHEMICALS IN THE WATER 109 SHOWS THE WATER DRAGON SYSTEM DESTROYING HEAVY METALS AND CHEMICALS AND 102 DESTROYING ANY TOXIC GASES OR CO₂
PART NO. 117 SHOWS THE NOW CLEAN WATER RE-ENTERING THE STACK VIA SPRAYS REABSORBING THE HEAVY METALS AND CHEMICAL FOR RETURN TO THE SLUDGE CHAMBER AND THE PROCESS CONTINUES
PART NO. 118 LIFTING PLATFORM OR CONTAINER TO HOLD OZONE OR AIR BUBBLERS OR MICRO BUBBLERS AND ULTRAVIOLET BULBS HOUSED IN QUARTZ TUBES FOR RAISING AND LOWERING INTO WATER SOLUTION OR GASES.
PART NO. 119 LIFTING PLATFORM BASE
PART NO. 120 LIFTING PLATFORM JOIN
PART NO. 121 ROWS OF ULTRAVIOLET BULBS IN QUARTZ TUBES
PART NO. 122 SHIPPING CONTAINER FOR HOUSING OZONE GENERATORS AIR PUMPS AND GENERATORS WHICH SUPPORT LIFTING PLATFORM IN PART NO. 118
PART NO. 123 TOP VIEW OF UV IN QUARTZ TUBES SET IN ROWS THE DISTANCE BETWEEN EACH ROW CAN BE VARIED TO CHANGE THE REACTIONS AS WATER IS PUSHED OR DRIVEN THROUGH THE PLATFORM
PART NO. 124 LIFTING OR LOWERING PLATFORM
PART NO. 125 END VIEW LIFTING AND LOWERING THE PLATFORM WITH VERTICAL ROWS OF UV IN QUARTZ TUBES.
PART NO. 126 PADDLE WHEEL TO PUSH OR PULL WATER OR FLUIDS THROUGH THE PLATFORM WHEN SUBMERGED OR DRIVE THE VESSEL THROUGH THE WATER
PART NO. 127 BEARING AND BEARING HOUSING TO HOLD DRIVE SHAFT WHICH TURNS THE PADDLES
PART NO. 128 SHAFTS ATTACHED TO THE PADDLES
PART NO. 129 HOUSING TO HOLD QUARTZ TUBES IN PLACE
PART NO. 130 END VIEWS OF AIR STONES AND MICRO BUBBLERS ATTACHED TO THE BASE OF LIFTING PLATFORM
PART NO. 131 OZONE OR AIR DELIVERY PIPE
PART NO. 132 LIFTING PLATFORM CENTER JOIN
PART NO. 133 FRONTS OR BOW SECTION BOLTED TO THE FRONT OF SHIPPING CONTAINER NOW SEALED TO BECOME A CATAMARAN
PART NO. 134 STERN SECTION BOLTS ON TO CONTAINER TO HOLD PADDLE AND SHAFT IN POSITION
PART NO. 135 WINDOWS
PART NO. 136 JOIN SECTIONS TO HOLD BOLT ON BOW AND STERN SECTION
PART NO. 137 LIGHTS ON
PART NO. 138 LIGHTS OFF THIS CHANGED THE POLARIZATION AND ADVANCED OXIDATION REACTIONS
PART NO. 139 SHOWING THE LIGHTS IN A COMPACT SETTING TO CHANGE REACTION.
PART NO. 140 CROSS SECTIONS TO HOLD THE TWIN HULLS TOGETHER FRONT AND REAR
PART NO. 141 LIFTING PLATFORM IN RAISED POSITION
PART NO. 142 LIFTING PLATFORM IN THE LOWERED OPERATING POSITIONS PART NO. 143 WATER FLOW
PART NO. 144 SIDE VIEW OF UV IN QUARTZ TUBES
PART NO. 145 SIDE VIEW OF WATER PADDLE
PART NO. 146 SIDE VIEW OF AIRSTONES/VENTURES MICRO BUBBLERS
PART NO. 147 DOUBLE JOINED CONTAINERS
PART NO. 148 SPRAY PIPE TO SPRAY WATER OVER BACTERIAL MEDIA PLACED ON TOP OF AIR STONES
PART NO. 149 SEALED AND FIXED PLATFORM STACKED 2 HIGH
PART NO. 150 LIFTING PLATFORM IN THE UP POSITION
PART NO. 151 LIFTING PLATFORM IN THE SUBMERGED WORMING POSITION
PART NO. 152 BIOLOGICAL MEDIA
PART NO. 153 SHORT REFLECTIVE PLATE TO REFLECT LIGHT WAVES CAN BE WHITE OR MIRROR FINISH.
PART NO. 154 LONG DIRECTIONAL REFLECTIVE PLATES TO REFLECT LIGHT FOR POLARIZATION ENHANCEMENT.
PART NO. 155 VESSEL WATER LINE
PART NO. 156 FLEXIBLE OZONE PIPE
PART NO. 157 FLUID TRANSFER PIPES TO ALLOW SECTION TO BE JOINED TOGETHER AND FLOW INLET & EXIT.
PART NO. 158 WATER FLOWS UNDER.
PART NO. 159 WATER FLOWS OVER.
PART NO. 160 ADJUSTABLE FRACTIONATION REMOVAL PIPES OR COLLECTORS.
PART NO. 161 TOP SECTION OF TREATMENT CHAMBER HOUSING.
PART NO. 162 MAIN BODY OF TREATMENT CHAMBER HOUSING.
PART NO. 163 FLOW CONTROL PANELS IN SINGLE SECTION AND SEPARATE PANELS WHICH JOIN TOGETHER TO MAKE ONE SINGLE PANEL.
PART NO. 164 SHOW SLIDE GUIDE SECTION. THIS SECTION ALLOWS THE FLOW CONTROL PANEL NO. 163 TO BE RELOCATED IS REQUIRED. THIS ALLOWS FOR HUNDREDS OF DIFFERING REACTIONS.
PART NO. 165 AIR COMPRESSORS AND BLOWER
PART NO. 166 AIR DELIVERY PIPE TO OZONE CHAMBERS
PART NO. 167 OZONE PRODUCING TUBES
PART NO. 168 ELECTRIC MOTOR
PART NO. 169 OZONE DELIVERY PIPES OR DISTRIBUTION
PART NO. 170 OZONE FORMATION CHAMBERS
PART NO. 171 AIRS OR PURE OXYGEN DELIVERY TUBE
PART NO. 172 SHIPPING CONTAINER OR SIMILAR STACK OF BUILDING
PART NO. 173 CLEAN WATER DELIVERY PIPE TO TURBINE
PART NO. 174 10 FT. CONTAINER
PART NO. 175 TURBINES AND GENERATOR
PART NO. 176 PURIFIED WATER STORAGE CONTAINER
PART NO. 177 CONTROL FLOW VALVES
PART NO. 178 FLEXIBLE RUBBER STRIPS TO PREVENT LIGHT MOVING TO ONE CHAMBER TO ANOTHER BUT ALLOWING FLUID TO MOVE FROM ONE CHAMBER TO ANOTHER THROUGH THE STRIPS
PART NO. 179 CONCRETE OR FIX BARRIER
PART NO. 180 RECTANGULAR HOUSING PART NO. 181 SEALED CAP OR VALVE
PART NO. 182 CLEAN WATER COLLECTION TANK
PART NO. 183 DRUM ROLLERS
PART NO. 184 FINE SCREEN BELT
PART NO. 185 INPUT WATER
PART NO. 186 DRIVE SPROCKETS
PART NO. 187 WASH DOWN SPRAY OR COMPRESSED AIR
PART NO. 188 WASTE PIPES
PART NO. 189 ROUND CONTINUOUS COLD FUSION REACTOR
PART NO. 190 CONTINUOUS C-SECTION COLD FUSION REACTOR
PART NO. 191 PADDLES DRYER
PART NO. 192 AUGERS TO CONVEYER SEWAGE SOLIDS TO DRYER
PART NO. 193 LONG ROTATING DRUM FILTER TO SEPARATE SOLIDS TO LIQUID
PART NO. 194 FILTERS
PART NO. 195 TRAY
PART NO. 196 POLLUTED WASTE LIQUID
PART NO. 197 BASE TRAY
PART NO. 198 CLEAN WATER PIPE
PART NO. 199 BELT SUPPORT PANEL
PART NO. 200 SUPPORT ROLLERS
PART NO. 201 WATER PUMP
PART NO. 202 SUCTION PIPE
PART NO. 203 PROCESSED WATER DELIVERY PIPE
PART NO. 204 CONTAINER FILTER
PART NO. 205 FILTERED WATER DELIVERY PIPE
PART NO. 206 RAW WATER INLET DELIVERY PIPE
PART NO. 207 20 FT. CONTAINER
PART NO. 208 40 FT. CONTAINER
PART NO. 209 PROCESSED WATER LEVEL
PART NO. 210 VALVE OPEN
PART NO. 211 VALVE CLOSED
PART NO. 212 PROPELLER FOR PUSHING THE WATER
PART NO. 213 FISH OR AQUITE ANIMALS
PART NO. 214 WATER
PART NO. 215 STEPS
PART NO. 216 WALKWAY
PART NO. 217 INTERNAL TANK
PART NO. 218 TEMPERATURE CONTROL
PART NO. 219 AMMONIA REDUCTION STATION
PART NO. 220 CONTROL FISH CONTAINMENT
PART NO. 221 OZONE/OZENE FOR DISTRIBUTION TO DIFFUSERS
PART NO. 222 WASTE WATER AND GAS REMOVAL CONTAIN
PART NO. 223 RECIRCULATING WATER
PART NO. 224 INDUSTRIAL LIFT
PART NO. 225 ELECTRICAL WIRING TO BALLAST AND UV LIGHTS
PART NO. 226 ELECTRICAL COVERS WITH BALLAST AND UV LIGHTS UNDER
PART NO. 227 TANK WITH OZENE BUBBLES PASSING UV LIGHTS POLARIZATION
PART NO. 228 SINGLE OR JOINED REACTION CHAMBER
PART NO. 229 INSPECTION HATCH
PART NO. 230 AIR PASSES UNDER FOR COOLING
PART NO. 231 WATER PROOF AND SECURITY COVER IN LOCKABLE POSITION
PART NO. 232 MESH PROTECTION OF AIR PUMP FOR OZENE PRODUCTION FROM 4 FRAME TUBES HOUSING OF OZONE/OZENE PRODUCING REACTION CHAMBERS
PART NO. 233 STAINLESS STEEL OZENE REACTION CHAMBER
PART NO. 234 BALLAST FOR UV OR OZONE
PART NO. 235 OZENE PRODUCTION
PART NO. 236 AIR INTO OZENE REACTION CHAMBER
PART NO. 237 FRAME BECOMES OZENE REACTION CHAMBER
PART NO. 238 ROLLER SHUTTER/ROLLER SHUTTER TO BE FITTED FOR ELECTRICAL CABINET
PART NO. 239 OSCILLATING FAN FOR COOLING
PART NO. 240 OZENE REACTION CHAMBERS
PART NO. 241 OUTFLOW CLEAN WATER CONTROLS THE WATER HEIGHT
PART NO. 242 VERTICAL QUARTZ TUBE WITH OZONE BULBS
PART NO. 243 OUTFLOW AT HE BASE OF THE REACTION CHAMBER
PART NO. 244 USED OZENE/OZONE GAS EXHAUST EXIT CAN BE MIXED WITH ADDITIONAL AIR FOR QUICK BREAKDOWN TO USABLE CLEAN AIR IN MINUTES AS WELL A 254-NM UV LIGHT WILL ALSO ASSIST IN QUICK BREAKDOWN OF OZENE GAS
PART NO. 245 EXHAUST GASES AND FLUIDS VACUUM VENTURI OR NATURAL FLOW OF WASTE WATER AND GASES CAN BE WELDED TOGETHER TO JOIN UP TO ANY NUMBER OF REACTION WITH JOINING PIPES FROM BOTTOM TO TOP TO TRANSFER WATER FROM CHAMBER TO CHAMBER INTERNALLY OR EXTERNALLY
PART NO. 246 REACTION CHAMBERS JOINED TOGETHER INTO MULTIPLE CHAMBERS

1. A waste treatment system including at least one treatment chamber through which a fluid to be treated is moved in a fluid flow, the at least one treatment chamber including:
i. a plurality of ultraviolet light sources configured to irradiate fluid in the at least one treatment chamber with light at a wavelength of from 240 nm to 400 nm; and
ii. at least one dispersion assembly located at a base of the treatment chamber to disperse ozone supplied to the treatment chamber in a counter flow direction to the fluid flow; and
at least one ozone generation chamber configured to supply ozone to the at least one dispersion assembly, wherein the at least one ozone generation chamber includes a gas inlet for introduction of an oxygen-containing gas, and at least one ultraviolet light source, wherein the at least one ultraviolet light source is configured to irradiate oxygen-containing gas in the at least one ozone generation chamber with light at a wavelength of from 50 nm to 240 nm to thereby produce ozone.

2. A waste treatment system as claimed in claim 1 wherein the plurality of ultraviolet light sources in the at least one treatment chamber is at least ten ultraviolet light sources.

3. A waste treatment system as claimed in claim 1 wherein the plurality of ultraviolet light sources in the at least one treatment chamber are configured to irradiate fluid in the at least one treatment chamber with light at a wavelength of from 240 nm to 260 nm.

4. A waste treatment system as claimed in claim 1 wherein the at least one ultraviolet light source in the at least one ozone generation chamber is at least ten ultraviolet light sources.

5. A waste treatment system as claimed in claim 1 wherein the at least one ultraviolet light source in the at least one ozone generation chamber is configured to irradiate oxygen-containing gas in the at least one ozone generation chamber with light at a wavelength of from 170 nm to 200 nm to thereby produce ozone.

6. A waste treatment system as claimed in claim 1 wherein the UV light sources are vertically oriented and/or horizontally oriented in the at least one treatment chamber.

7. A waste treatment system as claimed in claim 1 wherein the at least one dispersion assembly disperses the ozone in bubbles having a diameter of less than 100 μm.

8. A waste treatment system as claimed in claim 1 wherein the internal surface of the at least one treatment chamber is reflective to ultraviolet light provided by the plurality of ultraviolet light sources.

9. A waste treatment system as claimed in claim 1 wherein the at least one treatment chamber further includes at least one suction source for reducing the pressure within the at least one treatment chamber.

10. A waste treatment system as claimed in claim 1 wherein the at least one ozone generation chamber is configured to supply gas including at least 10 ppm ozone to the at least one dispersion assembly.

11. A waste treatment system as claimed in claim 1 wherein the at least one treatment chamber is a plurality of treatment chambers, wherein fluid to be treated flows through at least two of said plurality of treatment chambers.

12. A waste treatment system as claimed in claim 1 wherein the at least one treatment chamber is at least five treatment chambers, wherein fluid to be treated flows sequentially through the at least five treatment chambers.

13. A waste treatment system as claimed in claim 11 wherein the system includes at least one connector conduit to an outlet at a base of a first treatment chamber with an inlet at an upper position of a second, adjacent treatment chamber.

14. A waste treatment system as claimed in claim 11 wherein the flow direction of the fluid to treated through each treatment chamber is in a direction which is opposite to the direction in which the ozone is dispersed into the treatment chamber.

15. A waste treatment system as claimed in claim 13 wherein one or more chlorinators are provided within the connector conduit such that fluid flowing through the connector conduit is treated with chlorine produced by the one or more chlorinators.

16. A waste treatment system as claimed in claim 1 wherein the system further includes at least one ozone treatment chamber through which a fluid to be treated is moved in a fluid flow, the at least one ozone treatment chamber including:
at least one dispersion assembly located at a base of the ozone treatment chamber to disperse ozone supplied to the ozone treatment chamber in a counter flow direction to the fluid flow;
wherein the at least one ozone generation chamber is configured to supply ozone to the at least one dispersion assembly in the ozone treatment chamber;
wherein fluid to be treated moves through the at least one treatment chamber and the at least one ozone treatment chamber.

17. A waste treatment system as claimed in claim 16, wherein fluid to be treated moves through the at least one ozone treatment chamber before moving through the at least one treatment chamber.

18. A waste treatment system as claimed in claim 16 wherein the system further includes at least one ultraviolet light treatment chamber through which a fluid to be treated is moved in a fluid flow, the at least one ultraviolet light treatment chamber including:
a plurality of ultraviolet light sources configured to irradiate fluid in the at least one treatment chamber with light at a wavelength of from 240 nm to 400 nm; and
wherein fluid to be treated moves through the at least one treatment chamber and the at least one ultraviolet light treatment chamber.

19. A waste treatment system as claimed in claim 18, wherein fluid to be treated moves through the at least one treatment chamber before moving through the at least one ultraviolet light treatment chamber.

20. A waste treatment system as claimed in claim 19 wherein the fluid moving through the at least one treatment chamber is polarised.

21. A method of treating a fluid, the method including passing fluid through the waste treatment system of claim 1.

22. A waste treatment system including
at least one treatment chamber through which a fluid to be treated is moved in a fluid flow, the or each treatment chamber including at least one of:
i. a number of ultraviolet light sources; and
ii. at least one dispersion assembly located at a base of the treatment chamber to disperse at least one sterilizing agent supplied to the treatment chamber in a counter flow direction to the fluid flow.
